(19) United States
(12) Patent Application Publication
(10) Pub. No.: US 2026/0274876 A1
(43) Pub. Date: Sep. 17, 2026

(54) 3-PYRROLIDINE-INDOLE DIMERS AS SEROTONERGIC AGENTS USEFUL FOR THE TREATMENT OF DISORDERS RELATED THERETO

(71) Applicant: MINDSET PHARMA INC., St. Laurent (CA)

(72) Inventors: Abdelmalik SLASSI, Mississauga (CA); Joseph A. ARAUJO, Grimsby (CA)

(21) Appl. No.: 19/100,309

(22) PCT Filed: Aug. 4, 2023
(86) PCT No.: PCT/CA2023/051049

§ 371 Date: Jan. 31, 2025

Related U.S. Application Data
(60) Provisional application No. 63/395,534, filed on Aug. 5, 2022.

(51) Int. Cl. C07F 9/6558; A61K 31/404; A61K 31/675; C07B 59/00; C07D 403/14

(52) U.S. Cl. CPC C07F 9/65583; A61K 31/404; A61K 31/675; C07B 59/002; C07B 59/004; C07D 403/14; C07B 2200/05

(57) ABSTRACT

The present application relates to 3-cyclic amine-indole dimers of general Formula (I), to processes for their preparation, to compositions comprising them and to their use in activation of a serotonin receptor in a cell, as well as to treating diseases, disorders or conditions by activation of a serotonin receptor in or on a cell. The diseases, disorders or conditions include, for example, psychosis, mental illnesses and CNS disorders. I

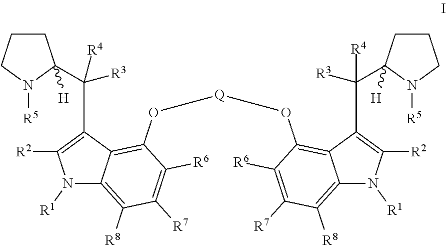

### RELATED APPLICATIONS

The present application claims the benefit of priority of co-pending U.S. provisional patent application No. 63/395,534 filed on Aug. 5, 2022 the contents of which are incorporated herein by reference in their entirety.

### FIELD

The application relates to 3-pyrrolidine-indole dimers of general Formula (I) for the treatment of different conditions that are treated by activation of serotonin receptors, for example, mental illnesses and neurological disease, in the fields of psychiatry, neurobiology and pharmacotherapy.

### BACKGROUND OF THE APPLICATION

Mental health disorders, or mental illness, refer to a wide range of disorders that include, but are not limited to, depressive disorders, anxiety and panic disorders, schizophrenia, eating disorders, substance misuse disorders, post-traumatic stress disorder, attention deficit/hyperactivity disorder and obsessive-compulsive disorder. Many mental health disorders, as well as neurological disorders, are impacted by alterations, dysfunction, degeneration, and/or damage to the brain's serotonergic system, which may explain, in part, common endophenotypes and comorbidities among neuropsychiatric and neurological diseases.

The field of psychedelic neuroscience has witnessed a recent renaissance following decades of restricted research due to their legal status. Psychedelics (serotonergic hallucinogens) are powerful psychoactive substances that alter perception and mood and affect numerous cognitive processes. Today there is a consensus that psychedelics are agonists or partial agonists at serotonin 5-hydroxytryptamine 2A (5-HT2A) receptors.

Psychedelics have both rapid onset and persisting effects long after their acute effects, which includes changes in mood and brain function. Long lasting effects may result from their unique receptor affinities, which affect neurotransmission via neuromodulatory systems that serve to modulate brain activity, i.e., neuroplasticity, and promote cell survival, are neuroprotective, and modulate brain neuroimmune systems. The mechanisms which lead to these long-term neuromodulatory changes may be linked to epigenetic modifications, gene expression changes and modulation of pre- and post-synaptic receptor densities. These, previously under-researched, psychedelic drugs may potentially provide the next-generation of neurotherapeutics, where treatment resistant psychiatric and neurological diseases, e.g., depression, post-traumatic stress disorder, dementia and addiction, may become treatable with attenuated pharmacological risk profiles.

Although there is a general perception that psychedelic drugs are dangerous, from a physiologic safety standpoint, they are one of the safest known classes of CNS drugs. Preliminary data show that psychedelic administration in humans results in a unique profile of effects and potential adverse reactions that need to be appropriately addressed to maximize safety. The primary safety concerns are largely psychologic, rather than physiologic, in nature. Somatic effects vary but are relatively insignificant, even at doses that elicit powerful psychologic effects. Psilocybin, when administered in a controlled setting, has frequently been reported to cause transient, delayed headache, with incidence, duration, and severity increased in a dose-related manner [Johnson et al., Drug Alcohol Depend (2012) 123(1-3):132-140]. It has been found that repeated administration of psychedelics leads to a very rapid development of tolerance known as tachyphylaxis, a phenomenon believed to be mediated, in part, by 5-HT2A receptors. In fact, several studies have shown that rapid tolerance to psychedelics correlates with downregulation of 5-HT2A receptors. For example, daily LSD administration selectively decreased 5-HT2 receptor density in the rat brain [Buckholtz et al., Eur. J. Pharmacol. 1990, 109:421-425. 1985; Buckholtz et al., Life Sci. 1985, 42:2439-2445].

Classic psychedelics and dissociative psychedelics are known to have rapid onset antidepressant and anti-addictive effects, unlike any currently available treatment. Randomized clinical control studies have confirmed antidepressant and anxiolytic effects of classic psychedelics in humans.

Psilocybin (4-phosphoryloxy-N,N-dimethyltrypatmine) has the chemical formula C₁₂H₁₇N₂O₄P. It is a tryptamine-based prodrug and is one of the major psychoactive constituents in mushrooms of the psilocybe species. It was first isolated from psilocybe mushrooms by Hofmann in 1957, later synthesized by him in 1958 [Passie et al. Addict Biol., 2002, 7(4):357-364], and was used in psychiatric, psychological research and in psychotherapy during the early to mid-1960s up until its controlled drug scheduling in 1970 in the US, and up until the 1980s in Germany [Passie 2005; Passie et al. Addict Biol., 2002, 7(4):357-364]. Research into the effects of psilocybin resumed in the mid-1990s, and it is currently the preferred compound for use in studies of the effects of serotonergic hallucinogens [Carter et al. J. Cogn. Neurosci., 2005 17(10):1497-1508; Gouzoulis-Mayfrank et al. Neuropsychopharmacology 1999, 20(6):565-581; Hasler et al, Psychopharmacology (Berl) 2004, 172(2):145-156], likely because it has a shorter duration of action and suffers from less notoriety than LSD. Like other members of this class, psilocybin induces sometimes profound changes in perception, cognition and emotion, including emotional lability.

In humans as well as other mammals, psilocybin is transformed into the active metabolite psilocin, or the 4-hydroxy-N,N-dimethyltryptamine parent compound. It is likely that psilocin partially or wholly produces most of the subjective and physiological effects of psilocybin in humans and non-human animals. Recently, human psilocybin research confirmed the 5-HT2A activity of psilocybin via the parent psilocin, and provides some support for indirect effects on dopamine through 5-HT2A activity and possible activity at other serotonin receptors. In fact, the most consistent finding for involvement of other receptors in the actions of psychedelics is the 5-HT1A receptor. That is particularly true for tryptamines and LSD, which generally have significant affinity and functional potency at this receptor. It is known that 5-HT1A receptors are colocalized with 5-HT2A receptors on cortical pyramidal cells [Martin-Ruiz et al. J Neurosci. 2001, 21(24):9856-986], where the two receptor types have opposing functional effects [Araneda et al. Neuroscience 1991, 40(2):399-412].

Although the exact role of the 5-HT2A receptor, and other 5-HT2 receptor family members, is not well understood with respect to the amygdala, it is evident that the 5-HT2A receptor plays an important role in emotional responses and is an important target to be considered in the actions of 5-HT2A agonist psychedelics. In fact, a majority of known 5-HT2A agonists produce hallucinogenic effects in humans, and rodents generalize from one 5-HT2A agonist to others, as between psilocybin and LSD [Aghajanian et al., Eur J Pharmacol., 1999, 367(2-3):197-206; Nichols at al., J Neurochem., 2004, 90(3):576-584]. Psilocybin has a stronger affinity for the human 5-HT2A receptor than for the rat receptor and it has a lower K(i) for both 5-HT2A and 5-HT2C receptors than LSD. Moreover, results from a series of drug-discrimination studies in rats found that 5-HT2A antagonists, but not 5-HT1A antagonists, prevented rats from recognizing psilocybin [Winter et al., Pharmacol Biochem Behav., 2007, 87(4):472-480]. Daily doses of LSD and psilocybin reduce 5-HT2 receptor density in rat brain.

Today, psilocybin is one of the most widely used psychedelics in human studies due to its relative safety, moderately long active duration, and good absorption in subjects. There remains strong research and therapeutic potential for psilocybin as recent studies have shown varying degrees of success in neurotic disorders, alcoholism, depression associated with major depressive disorder, treatment resistant depression and in terminally ill cancer patients, obsessive compulsive disorder, addiction, anxiety, post-traumatic stress disorder and even cluster headaches.

Recent developments include several double-blind placebo-controlled phase 2 studies of psilocybin-assisted psychotherapy in patients with treatment resistant depression, major depressive disorder and cancer-related psychosocial distress that demonstrate unprecedented positive relief of anxiety and depression. Two recent small pilot studies of psilocybin-assisted psychotherapy also have shown positive benefit in treating both alcohol and nicotine addiction. Recently, blood oxygen level-dependent functional magnetic resonance imaging (fMRI) and magnetoencephalography (MEG) have been employed for in vivo brain imaging in humans after administration of a psychedelic, and results indicate that intravenously administered psilocybin and LSD produce decreases in oscillatory power in areas of the brain's default mode network [Nichols DE. Pharmacol Rev., 2016, 68(2):264-355].

Preliminary studies using positron emission tomography (PET) showed that psilocybin ingestion (15 or 20 mg orally) increased absolute metabolic rate of glucose in frontal, and to a lesser extent in other, cortical regions as well as in striatal and limbic subcortical structures in healthy participants, suggesting that some of the key behavioral effects of psilocybin involve the frontal cortex [Gouzoulis-Mayfrank et al., Neuropsychopharmacology, 1999, 20(6):565-581; Vollenweider et al., Brain Res. Bull. 2001, 56(5):495-507]. Although 5-HT2A agonism is widely recognized as the primary action of classic psychedelic agents, psilocybin has less affinity for a wide range of other pre- and post-synaptic serotonin and dopamine receptors, as well as the serotonin reuptake transporter [Tyls et al., Eur. Neuropsychopharmacol., 2014, 24(3):342-356]. Psilocybin activates 5-HT1A receptors, which may contribute to antidepressant/anti-anxiety effects.

Depression and anxiety are two of the most common psychiatric disorders worldwide. Depression is a multifaceted condition characterized by episodes of mood disturbances alongside other symptoms such as anhedonia, psychomotor complaints, feelings of guilt, attentional deficits and suicidal tendencies, all of which can range in severity. Similarly, anxiety disorders are a collective of etiologically complex disorders characterized by intense psychosocial distress and other symptoms depending on the subtype. Anxiety associated with life-threatening disease is the only anxiety subtype that has been studied in terms of psychedelic-assisted therapy. Pharmacological and psychosocial interventions are commonly used to manage this type of anxiety, but their efficacy is mixed and limited such that they often fail to provide satisfactory emotional relief. Recent interest into the use of psychedelic-assisted therapy may represent a promising alternative for patients with depression and anxiety that are ineffectively managed by conventional methods.

Generally, the psychedelic treatment model consists of administering the orally-active drug to induce a mystical experience lasting approximately 4-9 h depending on the psychedelic [Halberstadt, Behav Brain Res., 2015, 277:99-120; Nichols, Pharmacol Rev., 2016, 68(2): 264-355]. This enables participants to work through and integrate difficult feelings and situations, leading to enduring anti-depressant and anxiolytic effects. Classical psychedelics like psilocybin and LSD are being studied as potential candidates. In one study with classical psychedelics for the treatment of depression and anxiety associated with life-threatening disease, it was found that, in a supportive setting, psilocybin, and LSD consistently produced significant and sustained anti-depressant and anxiolytic effects.

Psychedelic treatment is generally well-tolerated with few if any persisting adverse effects. Regarding their mechanisms of action, they mediate their main therapeutic effects biochemically via serotonin receptor agonism, and psychologically by generating meaningful psycho-spiritual experiences that contribute to mental flexibility. Given the limited success rates of current treatments for anxiety and mood disorders, and considering the high morbidity associated with these conditions, there is potential for psychedelics to provide symptom relief in patients inadequately managed by conventional methods.

Further emerging clinical research and evidence suggest psychedelic-assisted therapy, also shows potential as an alternative treatment for refractory substance use disorders and mental health conditions, and thus may be an important tool in a crisis where existing approaches have yielded limited success [dos Santos et al., Ther Adv Psychopharmacol., 2016, 6(3):193-213]. Similarly encouraging, are findings from a recent pilot study of psilocybin-assisted therapy for tobacco use disorder, demonstrating abstinence rates of 80% at six months follow-up and 67% at 12 months follow-up [Johnson et al., https://www.ncbi.nlm.nih.gov/pubmed/27441452 J Drug Alcohol Abuse, 2017, 43(1):55-60; Johnson et al., Psychopharmacol. 2014, 28(11):983-992]; such rates are considerably higher than any documented in the tobacco cessation literature. Notably, mystical-type experiences generated from the psilocybin sessions were significantly correlated with positive treatment outcomes. These results coincide with bourgeoning evidence from recent clinical trials lending support to the effectiveness of psilocybin-assisted therapy for treatment-resistant depression and end-of-life anxiety [Carhart-Harris et al. Neuropsychopharmacology, 2017, 42(11):2105-2113]. Research on the potential benefits of psychedelic-assisted therapy for opioid use disorder (OUD) is beginning to emerge, and accumulating evidence supports a need to advance this line of investigation. Available evidence from earlier randomized clinical trials suggests a promising role for treating OUD: higher rates of abstinence were observed among participants receiving high dose LSD and ketamine-assisted therapies for heroin addiction compared to controls at long-term follow-up. Recently, a large United States population study among 44,000 individuals found that psychedelic use was associated with 40% reduced risk of opioid abuse and 27% reduced risk of opioid dependence in the following year, as defined by DSM-IV criteria [Pisano et al., J Psychopharmacol., 2017, 31(5):606-613]. Similarly, a protective moderating effect of psychedelic use was found on the relationship between prescription opioid use and suicide risk among marginalized women [Argento et al., J Psychopharmacol., 2018, 32(12):1385-1391]. Despite the promise of these preliminary findings with classical psychedelic agents, further research is warranted to determine how psychedelics may ameliorate the opioid crisis. Meanwhile, growing evidence on the safety and efficacy of psilocybin for the treatment of mental and substance use disorders should help to motivate further clinical investigation into its use as a novel intervention for OUD.

Regular doses of psychedelics also ameliorate sleep disturbances, which are highly prevalent in depressed patients with more than 80% of patients having complaints of poor sleep quality. The sleep symptoms are often unresolved by first-line treatment and are associated with a greater risk of relapse and recurrence. Interestingly, sleep problems often appear before other depression symptoms, and subjective sleep quality worsens before the onset of an episode in recurrent depression. Two other studies assessing electroencephalographic (EEG) brain activity during sleep showed that psychedelics, such as LSD, positively affect sleep patterns. It further was suggested that a single dose of a psychedelic causes a reset of the biological clock underlying sleep/wake cycles and thereby enhances cognitive-emotional processes in depressed people but also improved feelings of well-being and enhanced mood in healthy individuals [Kuypers, Medical Hypotheses, 2019, 125:21-24].

In a systematic meta-analysis of clinical trials from 1960-2018 researching the therapeutic use of psychedelic treatment in patients with serious or terminal illnesses and related psychiatric illness, it was found that psychedelic therapy (mostly with LSD) may improve cancer-related depression, anxiety, and fear of death. Four randomized controlled clinical trials were published between 2011 and 2016, mostly with psilocybin treatment, that demonstrated psychedelic-assisted treatment can produce rapid, robust, and sustained improvements in cancer-related psychological and existential distress [Ross S, Int Rev Psychiatry, 2018, 30(4):317-330]. Many patients facing cancer or other life-threatening illnesses experience significant existential distress related to loss of meaning or purpose in life, which can be associated with hopelessness, demoralization, powerlessness, perceived burdensomeness, and a desire for hastened death. Those features are also often at the core of clinically significant anxiety and depression, and they can substantially diminish quality of life in this patient population. The alleviation of these core features of existential distress should be among the central aims of palliative care. Accordingly, several manualized psychotherapies for cancer-related existential distress have been developed in recent years, with an emphasis on dignity and meaning-making. However, there are currently no pharmacologic interventions for existential distress per se, and available pharmacologic treatments for depressive symptoms in patients with cancer have not demonstrated superiority over placebo. There remains a need for additional effective treatments for those conditions [Rosenbaum et al., Curr. Oncol., 2019, 26(4): 225-226].

Recently, there has been growing interest in a new dosing paradigm for psychedelics, such as psilocybin and LSD, referred to colloquially as microdosing. Under this paradigm, sub-perceptive doses of the serotonergic hallucinogens, approximately 10% or less of the full dose, are taken on a more consistent basis of once each day, every other day, or every three days, or a permutation of the same. Not only is this dosing paradigm more consistent with current standards in pharmacological care, but may be particularly beneficial for certain conditions, such as Alzheimer's disease, other neurodegenerative diseases, attention deficit disorder, attention deficit hyperactivity disorder, and for certain patient populations such as elderly, juvenile and patients that are fearful of or opposed to psychedelic assisted therapy. Moreover, this approach may be particularly well suited for managing cognitive deficits and preventing neurodegeneration. For example, subpopulations of low attentive and low motivated rats demonstrate improved performance on the 5-choice serial reaction time and progressive ratio tasks, respectively, following doses of psilocybin below the threshold for eliciting the classical wet dog shake behavioral response associated with hallucinogenic doses (Blumstock et al., WO 2020/157569 A1). Similarly, treatment of patients with hallucinogenic doses of 5-HT2A agonists is associated with increased BDNF and activation of the mTOR pathway, which are thought to promote neuroplasticity and are hypothesized to serve as molecular targets for the treatment of dementias and other neurodegenerative disorders (Ly et al. Cell Rep., 2018, 23(11):3170-3182). Additionally, several groups have demonstrated that low, non-hallucinogenic and non-psychomimetic, doses of 5-HT2A agonists also show similar neuroprotective and increased neuroplasticity effects (neuroplastogens) and reduced neuroinflammation, which could be beneficial in both neurodegenerative and neurodevelopmental diseases and chronic disorders (Manfredi et al., WO 2020/181194, Flanagan et al., Int. Rev. Psychiatry, 2018, 13:1-13; Nichols et al., 2016, Psychedelics as medicines; an emerging new paradigm). This repeated, lower, dose paradigm may extend the utility of these compounds to additional indications and may prove useful for wellness applications.

Psychosis is often referred to as an abnormal state of mind that is characterized by hallucinatory experiences, delusional thinking, and disordered thoughts. Moreover, this state is accompanied by impairments in social cognition, inappropriate emotional expressions, and bizarre behavior. Most often, psychosis develops as part of a psychiatric disorder, of which, it represents an integral part of schizophrenia. It corresponds to the most florid phase of the illness. The very first manifestation of psychosis in a patient is referred to as first-episode psychosis. It reflects a critical transitional stage toward the chronic establishment of the disease, that is presumably mediated by progressive structural and functional abnormalities seen in diagnosed patients. [ACS Chem. Neurosci., 2018, 9, 2241-2251]. Anecdotal evidence suggests that low, non-hallucinogenic, doses (microdosing) of psychedelics that are administered regularly can reduce symptoms of schizophrenia and psychosis.

### SUMMARY OF THE APPLICATION

The present application includes compounds of Formula I:

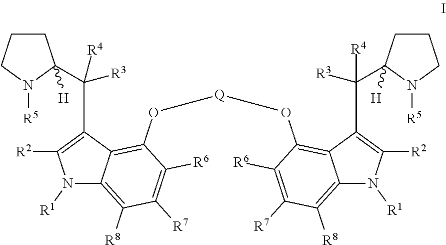

or a pharmaceutically acceptable salt, solvate and/or prodrug thereof,
wherein:
Q is selected from P(O)OR⁹, C₁-C₄alkylene-P(O)OR⁹—C₁-C₆alkylene, C(O), SO₂, C(O) Q′C(O), C(O)OQ′OC(O) and C(O)NR^{9′}Q′NR^{9′}C(O);
R¹ is selected from H, C₁-C₃alkyl, C(O)R¹⁰, CO₂R¹⁰, C(O)N(R¹⁰)(R¹¹), S(O)R¹⁰ and SO₂R¹⁰;
R², R³, R⁴ and R⁵ are independently selected from H and C₁-C₆alkyl;
R⁶, R⁷ and R⁸ are independently selected from H, halo, CN, OR¹², N(R¹²)(R¹³), SR¹², C₁-C₆alkyl, C₁-C₆haloalkyl, C₂-C₆haloalkenyl, CO₂R¹², C(O)N(R¹²)(R¹³), S(O)R¹², SO₂R¹², C₂-C₆alkenyl, C₂-C₆alkynyl, C₂-C₆haloalkynyl, C₃-C₇cycloalkyl and C₃-C₇heterocycloalkyl comprising 1 to 2 heteromoeities selected from O, S, S(O), SO₂, N and NR¹⁴, wherein said C₁-C₆alkyl, C₁-C₆haloalkyl, C₂-C₆alkenyl, C₂-C₆haloalkenyl, C₂-C₆alkynyl, C₂-C₆haloalkynyl, C₃-C₇cycloalkyl and C₃-C₇heterocycloalkyl are optionally substituted by one or more substituents independently selected from CN, OR¹⁵, N(R¹⁵)(R¹⁶) and SR¹⁵, and wherein said C₃-C₇cycloalkyl and C₃-C₇heterocycloalkyl are each further optionally substituted with one or more substituents selected from halo, CO₂R¹⁷, C(O)N(R¹⁷)(R¹⁸), SO₂R¹⁷, C₁-C₆alkyl, C₁-C₆haloalkyl, C₂-C₆alkenyl, C₂-C₆haloalkenyl, C₂-C₆alkynyl, C₂-C₆haloalkynyl, C₃-C₆cycloalkyl and C₃-C₆heterocycloalkyl comprising 1 to 2 heteromoeities selected from O, S, S(O), SO₂, N, and NR¹⁹;
Q′ is selected from a direct bond, C₁-C₂₀alkylene, C₁-C₂₀haloalkylene, C₂-C₂₀alkenylene, C₂-C₂₀haloalkenylene, C₂-C₂₀alkynylene, C₂-C₂₀haloalkynylene, C₃-C₇cycloalkylene, and C₃-C₇heterocycloalkylene comprising 1 to 2 heteromoeities selected from O, S, S(O), SO₂, N, and NR²⁰, wherein said C₁-C₂₀alkylene, C₂-C₂₀haloalkylene, C₂-C₆alkenylene, C₂-C₂₀haloalkenylene, C₃-C₇cycloalkylene, and C₃-C₇heterocycloalkylene are optionally substituted with one or more substituents independently selected from CN, OR²¹, N(R²¹)(R²²), and SR²¹, and/or are disubstituted on the same carbon atom with C₁₋₆alkyl, or with C₂₋₆alkylene to form a C₃-C₇cycloalkyl ring, and wherein said C₃-C₇cycloalkylene and C₃-C₇heterocycloalkylene are each further optionally substituted with one or more substituents selected from C₁-C₃alkyl and C₁-C₃haloalkyl, provided when Q is C(O)OQ′OC(O) or C(O)NR^{9′}Q′NR^{9′}C(O), then Q′ is not a direct bond;
each R⁹, R¹⁰, R¹¹, R¹², R¹³, R¹⁴, R¹⁵, R¹⁶, R¹⁷, R¹⁸ R¹⁹, R²⁰, R²¹ and R²² is independently selected from H, substituted or unsubstituted C₁-C₆alkyl, substituted or unsubstituted C₂-C₆alkenyl, substituted or unsubstituted C₂-C₆alkynyl, substituted or unsubstituted C₁-C₆haloalkyl, substituted or unsubstituted C₃-C₇cycloalkyl, substituted or unsubstituted C₃-C₇heterocycloalkyl, substituted or unsubstituted aryl, substituted or unsubstituted heteroaryl, substituted or unsubstituted C₁-C₆alkyleneC₃-C₇cycloalkyl, substituted or unsubstituted C₁-C₆alkyleneC₃-C₇heterocycloalkyl, substituted or unsubstituted C₁-C₆alkylenearyl, and substituted or unsubstituted C₁-C₆alkyleneheteroaryl; and
R^{9′} is independently selected from H and C₁-C₆alkyl;
wherein all available hydrogen atoms are optionally substituted with a fluorine or chlorine atom and/or all available atoms are optionally substituted with an alternate isotope thereof.

In a further embodiment, the compounds of the application are used as medicaments. Accordingly, the application also includes a compound of the application for use as a medicament.

The present application also includes a method of treating psychosis or psychotic symptoms comprising administering a therapeutically effective amount of one or more compounds of the application to a subject in need thereof.

The present application also includes a method of treating a mental illness comprising administering a therapeutically effective amount of one or more compounds of the application to a subject in need thereof.

The application additionally provides a process for the preparation of compounds of the application. General and specific processes are discussed in more detail below and set forth in the examples below.

Other features and advantages of the present application will become apparent from the following detailed description. It should be understood, however, that the detailed description and the specific examples, while indicating embodiments of the application, are given by way of illustration only and the scope of the claims should not be limited by these embodiments but should be given the broadest interpretation consistent with the description as a whole.

### DETAILED DESCRIPTION

### I. Definitions

Unless otherwise indicated, the definitions and embodiments described in this and other sections are intended to be applicable to all embodiments and aspects of the present application herein described for which they are suitable as would be understood by a person skilled in the art.

The term “compound(s) of the application” or “compound(s) of the present application” and the like as used herein refers to a compound of Formula I (including Formula IA, IB, IC, ID, IE and IF that fall within the scope of Formula I) and includes pharmaceutically acceptable salts, solvates and/or prodrugs thereof as well as all stereoisomers and regioisomers.

The term “composition(s) of the application” or “composition(s) of the present application” and the like as used herein refers to a composition, such a pharmaceutical composition, comprising one or more compounds of the application.

The term “and/or” as used herein means that the listed items are present, or used, individually or in combination. In effect, this term means that “at least one of or “one or more” of the listed items is used or present. The term “and/or” with respect to pharmaceutically acceptable salts and/or solvates thereof means that the compounds of the application exist as individual salts and solvates, as well as a combination of, for example, a salt of a solvate of a compound of the application.

As used in the present application, the singular forms “a”, “an” and “the” include plural references unless the content clearly dictates otherwise. For example, an embodiment including “a compound” should be understood to present certain aspects with one compound, or two or more additional compounds.

As used in this application and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The term “consisting” and its derivatives as used herein are intended to be closed terms that specify the presence of the stated features, elements, components, groups, integers and/or steps and also exclude the presence of other unstated features, elements, components, groups, integers and/or steps.

The term “consisting essentially of”, as used herein, is intended to specify the presence of the stated features, elements, components, groups, integers and/or steps as well as those that do not materially affect the basic and novel characteristic(s) of these features, elements, components, groups, integers and/or steps.

In embodiments comprising an “additional” or “second” component, such as an additional or second compound, the second component as used herein is chemically different from the other components or first component. A “third” component is different from the other, first and second components and further enumerated or “additional” components are similarly different.

The term “suitable” as used herein means that the selection of the particular compound or conditions would depend on the specific synthetic manipulation to be performed, the identity of the molecule(s) to be transformed and/or the specific use for the compound, but the selection would be well within the skill of a person trained in the art. All process/method steps described herein are to be conducted under conditions sufficient to provide the product shown. A person skilled in the art would understand that all reaction conditions, including, for example, reaction solvent, reaction time, reaction temperature, reaction pressure, reactant ratio and whether or not the reaction should be performed under an anhydrous or inert atmosphere, can be varied to optimize the yield of the desired product and it is within their skill to do so.

The terms “about”, “substantially” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies or unless the context suggests otherwise to a person skilled in the art.

The present description refers to a number of chemical terms and abbreviations used by those skilled in the art. Nevertheless, definitions of selected terms are provided for clarity and consistency.

The term “solvate” as used herein means a compound, or a salt and/or prodrug of a compound, wherein molecules of a suitable solvent are incorporated in the crystal lattice.

The term “prodrug” as used herein means a compound, or salt of a compound, that, after administration, is converted into an active drug.

The term “alkyl” as used herein, whether it is used alone or as part of another group, means straight or branched chain, saturated alkyl groups. The number of carbon atoms that are possible in the referenced alkyl group are indicated by the prefix “Cₙ₁₋ₙ₂”. Thus, for example, the term “C₁₋₆alkyl” (or “C₁-C₆alkyl”) means an alkyl group having 1, 2, 3, 4, 5, or carbon atoms.

The term “alkenyl” whether it is used alone or as part of another group, means a straight or branched chain, saturated alkylene group, that is, a saturated carbon chain that contains substituents on two of its ends. The number of carbon atoms that are possible in the referenced alkylene group are indicated by the prefix “Cₙ₁₋ₙ₂”. For example, the term C₂₋₆alkylene means an alkylene group having 2, 3, 4, 5 or 6 carbon atoms.

The term “alkynyl” as used herein, whether it is used alone or as part of another group, means straight or branched chain, unsaturated alkynyl groups containing at least one triple bond. The number of carbon atoms that are possible in the referenced alkyl group are indicated by the prefix “Cₙ₁₋ₙ₂”. For example, the term C₂₋₆alkynyl means an alkynyl group having 2, 3, 4, 5 or 6 carbon atoms.

As used herein, the term “alkoxy” as used herein, alone or in combination, includes an alkyl group connected to an oxygen-connecting atom.

The term “cycloalkyl,” as used herein, whether it is used alone or as part of another group, means a saturated carbocyclic group containing from 3 to 6 carbon atoms and one or more rings. The number of carbon atoms that are possible in the referenced cycloalkyl group are indicated by the numerical prefix “Cₙ₁₋ₙ₂”. For example, the term C₃₋ₗ₀cycloalkyl means a cycloalkyl group having 3, 4, 5 or 6 carbon atoms.

The term “heterocycloalkyl” as used herein, whether it is used alone or as part of another group, refers to cyclic groups containing at least one non-aromatic ring containing from 3 to 6 atoms in which one or more of the atoms are a heteromoiety selected from O, S, S(O), SO₂ and N and the remaining atoms are C. Heterocycloalkyl groups are either saturated or unsaturated (i.e. contain one or more double bonds). When a heterocycloalkyl group contains the prefix Cₙ₁₋ₙ₂ or “n1 to n2” this prefix indicates the number of carbon atoms in the corresponding carbocyclic group, in which one or more, suitably 1 to 4, of the ring atoms is replaced with a heteromoeity as selected from O, S, S(O), SO₂ and N and the remaining atoms are C.

The term “aryl” as used herein, whether it is used alone or as part of another group, refers to carbocyclic groups containing at least one aromatic ring and contains either 6 to 20 carbon atoms.

The term “heteroaryl” as used herein, whether it is used alone or as part of another group, refers to cyclic groups containing at least one heteroaromatic ring containing 5-6 atoms in which one or more of the atoms are a heteroatom selected from O, S and N and the remaining atoms are C. When a heteroaryl group contains the prefix Cₙ₁₋ₙ₂ this prefix indicates the number of carbon atoms in the corresponding carbocyclic group, in which one or more, suitably 1 to 4, of the ring atoms is replaced with a heteroatom as defined above.

All cyclic groups, including aryl, heteroaryl, heterocycloalkyl and cycloalkyl groups, contain one or more than one ring (i.e. are polycyclic). When a cyclic group contains more than one ring, the rings may be fused, bridged, spirofused or linked by a bond.

The term “benzofused” as used herein refers to a polycyclic group in which a benzene ring is fused with another ring.

A first ring being “fused” with a second ring means the first ring and the second ring share two adjacent atoms there between.

A first ring being “bridged” with a second ring means the first ring and the second ring share two non-adjacent atoms there between.

A first ring being “spirofused” with a second ring means the first ring and the second ring share one atom there between.

The term “halogen” (or “halo”) whether it is used alone or as part of another group, refers to a halogen atom and includes fluoro, chloro, bromo and iodo.

The term “haloalkyl” as used herein refers to an alkyl group as defined above in which one or more of the available hydrogen atoms have been replaced with a halogen. Thus, for example, “C₁₋₆haloalkyl” refers to a C₁ to C₆ linear or branched alkyl group as defined above with one or more halogen substituents.

As used herein, the term “haloalkenyl” refers to an alkenyl group as defined above in which one or more of the available hydrogen atoms have been replaced with a halogen. Thus, for example, “C₁₋₆haloalkenyl” (or “C₁-C₆haloalkenyl”) refers to a C₁ to C₆ linear or branched alkenyl group as defined above with one or more halogen substituents.

As used herein, the term “haloalkynyl” refers to an alkynyl group as defined above in which one or more of the available hydrogen atoms have been replaced with a halogen. Thus, for example, “C₁₋₆haloalkynyl” (or “C₁-C₆haloalkynyl”) refers to a C₁ to C₆ linear or branched alkynyl group as defined above with one or more halogen substituents.

The term “deuteroalkyl” as used herein refers to an alkyl group as defined above in which one or more of the available hydrogen atoms have been replaced with a deuterium. Thus, for example, “C₁₋₆deuteroalkyl” refers to a C₁ to C₆ linear or branched alkyl group as defined above with one or more deuterium substituents.

The suffix “ene” at the end of a group (for example “alkylene” or “alkenylene”) means that the group is bivalent, that is that it is bonded to two variables each on a different end of the group.

The term “optionally substituted” as used here means that the subject group is unsubstituted or substituted and the terms “optionally substituted” and “unsubstituted or substituted” as used interchangeably herein.

The term “substituted herein” means that one or more hydrogen atoms in the group are substituted or replaced with a substituent group independently selected from halo, C₁-C₄alkyl, OC₁-C₄alkyl, C₁-C₄haloalkyl, OC₁-C₄haloalkyl, CN, OH, NH₂, NH(C₁-C₄alkyl), N(C₁-C₄alkyl)(C₁-C₄alkyl), SC₁-C₄alkyl, S(O)C₁-C₄alkyl, SO₂C₁-C₄alkyl, CO₂H, CO₂C₁-C₄alkyl, C(O)NH2, C(O)NHC₁-C₄alkyl, C(O)N(C₁-C₄alkyl)(C₁-C₄alkyl), C₃-C₆cycloalkyl and a 3- to 6-membered heterocyclic ring including 1 to 2 ring heteromoeities selected from O, S, S(O), SO₂, N, NH and NC₁-C₄alkyl.

The term “available”, as in “available hydrogen atoms” or “available atoms” refers to atoms that would be known to a person skilled in the art to be capable of replacement by a substituent.

As used herein, the term “one or more” item includes a single item selected from the list as well as mixtures of two or more items selected from the list.

The term “alternate isotope thereof” as used herein refers to an isotope of an element that is other than the isotope that is most abundant in nature.

In the compounds of general Formula I and pharmaceutically acceptable salts and/or solvates thereof, the atoms may exhibit their natural isotopic abundances, or one or more of the atoms may be artificially enriched in a particular isotope having the same atomic number, but an atomic mass or mass number different from the atomic mass or mass number predominantly found in nature. The present disclosure is meant to include all suitable isotopic variations of the compounds of general Formula I and pharmaceutically acceptable salts and/or solvates thereof. For example, different isotopic forms of hydrogen (H) include protium (¹H), deuterium (²H) and tritium (³H). Protium is the predominant hydrogen isotope found in nature.

The term “compound” refers to the compound and, in certain embodiments, to the extent they are stable, any hydrate or solvate thereof. A hydrate is the compound complexed with water and a solvate is the compound complexed with a solvent, which may be an organic solvent or an inorganic solvent. A “stable” compound is a compound that can be prepared and isolated and whose structure and properties remain or can be caused to remain essentially unchanged for a period of time sufficient to allow use of the compound for the purposes described herein (e.g., therapeutic administration to a subject). The compounds of the present application are limited to stable compounds embraced by general Formula (I), or pharmaceutically acceptable salts and/or solvates thereof.

The term “pharmaceutically acceptable” means compatible with the treatment of subjects.

The term “pharmaceutically acceptable carrier” means a non-toxic solvent, dispersant, excipient, adjuvant or other material which is mixed with the active ingredient in order to permit the formation of a pharmaceutical composition, i.e., a dosage form capable of administration to a subject.

The term “pharmaceutically acceptable salt” means either an acid addition salt or a base addition salt which is suitable for, or compatible with, the treatment of subjects.

An acid addition salt suitable for, or compatible with, the treatment of subjects is any non-toxic organic or inorganic acid addition salt of any basic compound.

A base addition salt suitable for, or compatible with, the treatment of subjects is any non-toxic organic or inorganic base addition salt of any acidic compound.

The term “protecting group” or “PG” and the like as used herein refers to a chemical moiety which protects or masks a reactive portion of a molecule to prevent side reactions in those reactive portions of the molecule, while manipulating or reacting a different portion of the molecule. After the manipulation or reaction is complete, the protecting group is removed under conditions that do not degrade or decompose the remaining portions of the molecule. The selection of a suitable protecting group can be made by a person skilled in the art. Many conventional protecting groups are known in the art, for example as described in “Protective Groups in Organic Chemistry” McOmie, J. F. W. Ed., Plenum Press, 1973, in Greene, T. W. and Wuts, P. G. M., “Protective Groups in Organic Synthesis”, John Wiley & Sons, 3^{rd} Edition, 1999 and in Kocienski, P. Protecting Groups, 3rd Edition, 2003, Georg Thieme Verlag (The Americas).

The term “subject” as used herein includes all members of the animal kingdom including mammals, and suitably refers to humans. Thus, the methods of the present application are applicable to both human therapy and veterinary applications.

The term “treating” or “treatment” as used herein and as is well understood in the art, means an approach for obtaining beneficial or desired results, including clinical results. Beneficial or desired clinical results include, but are not limited to alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease and remission (whether partial or total), whether detectable or undetectable. “Treating” and “treatment” can also mean prolonging survival as compared to expected survival if not receiving treatment. “Treating” and “treatment” as used herein also include prophylactic treatment. For example, a subject with early cancer can be treated to prevent progression, or alternatively a subject in remission can be treated with a compound or composition of the application to prevent recurrence. Treatment methods comprise administering to a subject a therapeutically effective amount of one or more of the compounds of the application and optionally consist of a single administration, or alliteratively comprise a series of administrations.

As used herein, the term “effective amount” or “therapeutically effective amount” means an amount of one or more compounds of the application that is effective, at dosages and for periods of time necessary to achieve the desired result. For example, in the context of treating a disease, disorder or condition mediated or treated by agonism or activation of serotonergic receptors and downstream second messengers, an effective amount is an amount that, for example, increases said activation compared to the activation without administration of the one or more compounds.

“Palliating” a disease, disorder or condition means that the extent and/or undesirable clinical manifestations of a disease, disorder or condition are lessened and/or time course of the progression is slowed or lengthened, as compared to not treating the disorder.

The term “administered” as used herein means administration of a therapeutically effective amount of one or more compounds or compositions of the application to a cell, tissue, organ or subject.

The term “prevention” or “prophylaxis”, or synonym thereto, as used herein refers to a reduction in the risk or probability of a patient becoming afflicted with a disease, disorder or condition or manifesting a symptom associated with a disease, disorder or condition.

The “disease, disorder or condition” as used herein refers to a disease, disorder or condition treated or treatable by activation a serotonin receptor, for example 5-HT_{2A} and particularly using a serotonin receptor agonist, such as one or more compounds of the application herein described.

The term “treating a disease, disorder or condition by activation of a serotonin receptor” as used herein means that the disease, disorder or condition to be treated is affected by, modulated by and/or has some biological basis, either direct or indirect, that includes serotonergic activity, in particular increases in serotonergic activity. These diseases respond favourably when serotonergic activity associated with the disease, disorder or condition is agonized by one or more of the compounds or compositions of the application.

The term “activation” as used herein includes agonism, partial agonist and positive allosteric modulation of a serotonin receptor.

The terms “5-HT_{1A}” and “5-HT_{2A}” are used herein mean the 5-HT_{2A} and 5-HT_{2A} receptor subtypes of the 5-HT₂ serotonin receptor.

The term “therapeutic agent” as used herein refers to any drug or active agent that has a pharmacological effect when administered to a subject.

### II. Compounds

The Applicant has developed and prepared novel pyrrolidine-indole dimer compounds. In some embodiments, the dimer compounds metabolize in vivo to provide active metabolites. For example, in some embodiments, exemplary dimer compounds I-13 and I-122 metabolizes in vivo to provide the active metabolites of compounds 10 and 13 respectively, as described herein.

The present application includes compounds of Formula I:

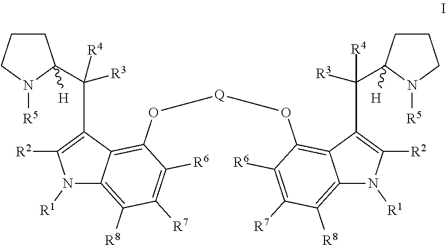

or a pharmaceutically acceptable salt, solvate and/or prodrug thereof,
wherein:
Q is selected from P(O)OR⁹, C₁-C₄alkylene-P(O)OR⁹—C₁-C₆alkylene, C(O), SO₂, C(O)Q′C(O), C(O)OQ′OC(O) and C(O)NR^{9′}Q′NR^{9′}C(O);
R¹ is selected from H, C₁-C₃alkyl, C(O)R¹⁰, CO₂R¹⁰, C(O)N(R¹⁰)(R¹¹), S(O)R¹⁰ and SO₂R¹⁰; y R², R³, R⁴ and R⁵ are independently selected from H and C₁-C₆alkyl;
R⁶, R⁷ and R⁸ are independently selected from H, halo, CN, OR¹², N(R¹²)(R¹³), SR¹², C₁-C₆alkyl, C₁-C₆haloalkyl, C₂-C₆haloalkenyl, CO₂R¹², C(O)N(R¹²)(R¹³), S(O)R¹², SO₂R¹², C₂-C₆alkenyl, C₂-C₆alkynyl, C₂-C₆haloalkynyl, C₃-C₇cycloalkyl and C₃-C₇heterocycloalkyl comprising 1 to 2 heteromoeities selected from O, S, S(O), SO₂, N and NR¹⁴, wherein said C₁-C₆alkyl, C₁-C₆haloalkyl, C₂-C₆alkenyl, C₂-C₆haloalkenyl, C₂-C₆alkynyl, C₂-C₆haloalkynyl, C₃-C₇cycloalkyl and C₃-C₇heterocycloalkyl are optionally substituted by one or more substituents independently selected from CN, OR¹⁵, N(R¹⁵)(R¹⁶) and SR¹⁵, and wherein said C₃-C₇cycloalkyl and C₃-C₇heterocycloalkyl are each further optionally substituted with one or more substituents selected from halo, CO₂R¹⁷, C(O)N(R¹⁷)(R¹⁸), SO₂R¹⁷, C₁-C₆alkyl, C₁-C₆haloalkyl, C₂-C₆alkenyl, C₂-C₆haloalkenyl, C₂-C₆alkynyl, C₂-C₆haloalkynyl, C₃-C₆cycloalkyl and C₃-C₆heterocycloalkyl comprising 1 to 2 heteromoeities selected from O, S, S(O), SO₂, N, and NR¹⁹;
Q′ is selected from a direct bond, C₁-C₂₀alkylene, C₁-C₂₀haloalkylene, C₂-C₂₀alkenylene, C₂-C₂₀haloalkenylene, C₂-C₂₀alkynylene, C₂-C₂₀haloalkynylene, C₃-C₇cycloalkylene, and C₃-C₇heterocycloalkylene comprising 1 to 2 heteromoeities selected from O, S, S(O), SO₂, N, and NR²⁰, wherein said C₁-C₂₀alkylene, C₂-C₂₀haloalkylene, C₂-C₆alkenylene, C₂-C₂₀haloalkenylene, C₃-C₇cycloalkylene, and C₃-C₇heterocycloalkylene are optionally substituted with one or more substituents independently selected from CN, OR²¹, N(R²¹)(R²²), and SR²¹, and/or are disubstituted on the same carbon atom with C₁₋₆alkyl, or with C₂₋₆alkylene to form a C₃-C₇cycloalkyl ring, and wherein said C₃-C₇cycloalkylene and C₃-C₇heterocycloalkylene are each further optionally substituted with one or more substituents selected from C₁-C₃alkyl and C₁-C₃haloalkyl, provided when Q is C(O)OQ′OC(O) or C(O)NR^{9′}Q′NR^{9′}C(O), then Q′ is not a direct bond;
each R⁹, R¹⁰, R¹¹, R¹², R¹³, R¹⁴, R¹⁵, R¹⁶, R¹⁷, R¹⁸ R¹⁹, R²⁰, R²¹ and R²² is independently selected from H, substituted or unsubstituted C₁-C₆alkyl, substituted or unsubstituted C₂-C₆alkenyl, substituted or unsubstituted C₂-C₆alkynyl, substituted or unsubstituted C₁-C₆haloalkyl, substituted or unsubstituted C₃-C₇cycloalkyl, substituted or unsubstituted C₃-C₇heterocycloalkyl, substituted or unsubstituted aryl, substituted or unsubstituted heteroaryl, substituted or unsubstituted C₁-C₆alkyleneC₃-C₇cycloalkyl, substituted or unsubstituted C₁-C₆alkyleneC₃-C₇heterocycloalkyl, substituted or unsubstituted C₁-C₆alkylenearyl, and substituted or unsubstituted C₁-C₆alkyleneheteroaryl; and
R^{9′} is independently selected from H and C₁-C₆alkyl;
wherein all available hydrogen atoms are optionally substituted with a fluorine or chlorine atom and/or all available atoms are optionally substituted with an alternate isotope thereof.

The present application includes compounds of Formula I:

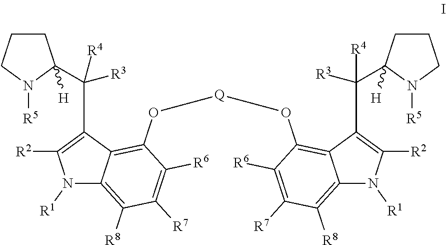

or a pharmaceutically acceptable salt, solvate and/or prodrug thereof, wherein:
Q is selected from P(O)OR⁹, C₁-C₄alkylene-P(O)OR⁹—C₁-C₆alkylene, C(O), SO₂ and C(O)Q′C(O);
R¹ is selected from H, C₁-C₃alkyl, C(O)R¹⁰, CO₂R¹⁰, C(O)N(R¹⁰)(R¹¹), S(O)R¹⁰ and SO₂R¹⁰;
R², R³, R⁴ and R⁵ are independently selected from H and C₁-C₆alkyl;
R⁶, R⁷ and R⁸ are independently selected from H, halo, CN, OR¹², N(R¹²)(R¹³), SR¹², C₁-C₆alkyl, C₁-C₆haloalkyl, C₂-C₆haloalkenyl, CO₂R¹², C(O)N(R¹²)(R¹³), S(O)R¹², SO₂R¹², C₂-C₆alkenyl, C₂-C₆alkynyl, C₂-C₆haloalkynyl, C₃-C₇cycloalkyl and C₃-C₇heterocycloalkyl comprising 1 to 2 heteromoeities selected from O, S, S(O), SO₂, N and NR¹⁴, wherein said C₁-C₆alkyl, C₁-C₆haloalkyl, C₂-C₆alkenyl, C₂-C₆haloalkenyl, C₂-C₆alkynyl, C₂-C₆haloalkynyl, C₃-C₇cycloalkyl and C₃-C₇heterocycloalkyl are optionally substituted by one or more substituents independently selected from CN, OR¹⁵, N(R¹⁵)(R¹⁶) and SR¹⁵, and wherein said C₃-C₇cycloalkyl and C₃-C₇heterocycloalkyl are each further optionally substituted with one or more substituents selected from halo, CO₂R¹⁷, C(O)N(R¹⁷)(R¹⁸), SO₂R¹⁷, C₁-C₆alkyl, C₁-C₆haloalkyl, C₂-C₆alkenyl, C₂-C₆haloalkenyl, C₂-C₆alkynyl, C₂-C₆haloalkynyl, C₃-C₆cycloalkyl and C₃-C₆heterocycloalkyl comprising 1 to 2 heteromoeities selected from O, S, S(O), SO₂, N, and NR¹⁹, Q′ is selected from a direct bond, C₁-C₂₀alkylene, C₁-C₂₀haloalkylene, C₂-C₂₀alkenylene, C₂-C₂₀haloalkenylene, C₂-C₂₀alkynylene, C₂-C₂₀haloalkynylene, C₃-C₇cycloalkylene, and C₃-C₇heterocycloalkylene comprising 1 to 2 heteromoeities selected from O, S, S(O), SO₂, N, and NR²⁰, wherein said C₁-C₂₀alkylene, C₂-C₂₀haloalkylene, C₂-C₆alkenylene, C₂-C₂₀haloalkenylene, C₃-C₇cycloalkylene, and C₃-C₇heterocycloalkylene are optionally substituted with one or more substituents independently selected from CN, OR²¹, N(R²¹)(R²²), and SR²¹, and/or are disubstituted on the same carbon atom with C₁₋₆alkyl, or with C₂₋₆alkylene to form a C₃-C₇cycloalkyl ring, and wherein said C₃-C₇cycloalkylene and C₃-C₇heterocycloalkylene are each further optionally substituted with one or more substituents selected from C₁-C₃alkyl and C₁-C₃haloalkyl; and
each R⁹, R¹⁰, R¹¹, R¹², R¹³, R¹⁴, R¹⁵, R¹⁶, R¹⁷, R¹⁸ R¹⁹, R²⁰, R²¹ and R²² is independently selected from H, substituted or unsubstituted C₁-C₆alkyl, substituted or unsubstituted C₂-C₆alkenyl, substituted or unsubstituted C₂-C₆alkynyl, substituted or unsubstituted C₁-C₆haloalkyl, substituted or unsubstituted C₃-C₇cycloalkyl, substituted or unsubstituted C₃-C₇heterocycloalkyl, substituted or unsubstituted aryl, substituted or unsubstituted heteroaryl, substituted or unsubstituted C₁-C₆alkyleneC₃-C₇cycloalkyl, substituted or unsubstituted C₁-C₆alkyleneC₃-C₇heterocycloalkyl, substituted or unsubstituted C₁-C₆alkylenearyl, and substituted or unsubstituted C₁-C₆alkyleneheteroaryl;
wherein all available hydrogen atoms are optionally substituted with a fluorine or chlorine atom and/or all available atoms are optionally substituted with an alternate isotope thereof.

In some embodiments, all available hydrogen atoms are optionally substituted with an alternate isotope thereof. In some embodiments, the alternate isotope of hydrogen is deuterium. Accordingly, in some embodiments, the compounds of the application are isotopically enriched with deuterium.

In some embodiments, R¹ is selected from H, C₁-C₃alkyl, C(O)R¹⁰, CO₂R¹⁰ and C(O)N(R¹⁰)(R¹¹), wherein all available hydrogen atoms are optionally substituted with a fluorine atom or chlorine atom and/or all available atoms are optionally substituted with an alternate isotope thereof. In some embodiments, R¹ is selected from H, C₁-C₃alkyl, C(O)R¹⁰ and CO₂R¹⁰, wherein all available hydrogen atoms are optionally substituted with a fluorine atom or chlorine atom and/or all available atoms are optionally substituted with an alternate isotope thereof. In some embodiments, R¹ is selected from is selected from H, CH₃ and CH₂CH₃, wherein all available hydrogen atoms are optionally substituted with a fluorine atom or chlorine atom and/or all available atoms are optionally substituted with an alternate isotope thereof. In some embodiments, R¹ is selected from is H, CH₃, CH₂CH₃, wherein all available hydrogen atoms are optionally and independently replaced with a fluorine atom or deuterium atom. In some embodiments, R¹ is selected from H, D, F, CH₃, CD₂H, CDH₂, CD₃, CF₃, CHF₂, CFH₂, CH₂CH₃, CH₂CH₂D, CH₂CD₂H and CD₂CD₃. In some embodiments, R¹ is independently selected from H, D, F, CH₃, CD₂H, CDH₂, CD₃, CF₃, CHF₂, CF₂H, CH₂CH₃, CH₂CH₂D, CH₂CD₂H and CD₂CD₃. In some embodiments, R¹ is selected from H, D, CH₃ and CD₃. In some embodiments, R¹ is selected from H, CH₃, CH₂CH₃, C(O)R¹⁰ and CO₂R¹⁰, wherein all available hydrogen atoms are optionally substituted with a fluorine atom or chlorine atom and/or all available atoms are optionally substituted with an alternate isotope thereof. In some embodiments, R¹ is selected from H, CH₃, and CH₂CH₃, wherein all available hydrogen atoms are optionally and independently replaced with a fluorine atom or deuterium atom. In some embodiments, R¹ is selected from H, D, F, CH₃, CD₂H, CDH₂, CD₃, CF₃, CHF₂, CF₂H, CH₂CH₃, CH₂CH₂D, CH₂CD₂H and CD₂CD₃ In some embodiments, R¹ is selected from H and D. In some embodiments, R¹ is H. In some embodiments, R¹ is selected from S(O)R¹⁰ and SO₂R¹⁰, wherein all available hydrogen atoms are optionally substituted with a fluorine atom or chlorine atom and/or all available atoms are optionally substituted with an alternate isotope thereof.

In some embodiments, R², R³ and R⁴ are independently selected from hydrogen and C₁-C₄alkyl, wherein all available hydrogen atoms are optionally substituted with a fluorine or chlorine atom and/or all available atoms are optionally substituted with an alternate isotope thereof. In some embodiments, R², R³ and R⁴ are independently selected from H, CH₃, CH₂CH₃, CH(CH₃)₂ and C(CH₃)₃, wherein all available hydrogen atoms are optionally substituted with a fluorine atom or chlorine atom and/or all available atoms are optionally substituted with an alternate isotope thereof. In some embodiments, R² is selected from hydrogen, CH₃, CH₂CH₃, CH(CH₃)₂ and C(CH₃)₃, wherein all available hydrogen atoms are optionally and independently replaced with a fluorine atom or deuterium atom. In some embodiments, R², R³ and R⁴ are independently selected from H, D, F, CH₃, CD₂H, CDH₂, CD₃, CF₃, CHF₂, CFH₂, CH₂CH₃, CH₂CH₂D, CH₂CD₂H and CD₂CD₃. In some embodiments, R² is selected from H, D, F, CH₃, CD₂H, CDH₂, CD₃, CF₃, CHF₂, CF₂H, CH₂CH₃, CH₂CH₂D, CH₂CD₂H and CD₂CD₃. In some embodiments, R² is selected from H, D, F, CH₃, CF₃, CH₂CH₃, CD₂CD₃, CF₂CF₃, CH(CH₃)₂, CD(CD₃)₂, CF(CF₃)₂, C(CD₃)₃, C(CF₃)₃ and C(CH₃)₃, In some embodiments, R² is selected from H and D. In some embodiments, R² is H.

In some embodiments, R³ and R⁴ are independently selected from H, CH₃, CH₂CH₃, CH(CH₃)₂ and C(CH₃)₃, wherein all available hydrogen atoms are optionally substituted with a fluorine atom or chlorine atom and/or all available hydrogen atoms are optionally substituted with deuterium. In some embodiments, R³ and R⁴ are independently selected from H, CH₃, CH₂CH₃, CH(CH₃)₂ and C(CH₃)₃, wherein all available hydrogen atoms are optionally and independently replaced with a fluorine atom or deuterium atom. In some embodiments, at least one of R³ and R⁴ is D or at least one of R³ and R⁴ comprises D. In some embodiments, R³ and R⁴ are independently selected from H, D, F, CH₃, CD₂H, CDH₂, CD₃, CF₃, CHF₂, CFH₂, CH₂CH₃, CH₂CH₂D, CH₂CD₂H. and CD₂CD₃. In some embodiments, R³ and R⁴ are independently selected from H, D, F, CH₃, CD₂H, CDH₂, CD₃, CF₃, CHF₂, CF₂H, CH₂CH₃, CH₂CH₂D, CH₂CD₂H. and CD₂CD₃. In some embodiments, R³ and R⁴ are independently selected from H, D, F, CH₃, CD₂H, CDH₂, and CD₃. In some embodiments, R³ and R⁴ are independently selected from H, D, F, CH₃, and CD₃. In some embodiments, R³ and R⁴ are independently selected from H, D and F. In some embodiments, at least one of R³ and R⁴ is F. In some embodiments, R³ and R⁴ are both H. In some embodiments, R³ and R⁴ are both F. In some embodiments, at least one of R³ and R⁴ is H. In some embodiments, R³ and R⁴ are both H. In some embodiments, R³ and R⁴ are both D.

In some embodiments, R⁵ is selected from H and C₁-C₄alkyl, wherein all available hydrogen atoms are optionally substituted with a fluorine atom or chlorine atom and/or all available atoms are optionally substituted with an alternate isotope thereof. In some embodiments, R⁵ is selected from H and C₁-C₄alkyl, wherein all available hydrogen atoms are optionally substituted with a fluorine atom or chlorine atom and/or all available hydrogen atoms are optionally substituted with deuterium. In some embodiments, R⁵ is selected from hydrogen, CH₃, CH₂CH₃, CH(CH₃)₂ and C(CH₃)₃, wherein all available hydrogen atoms are optionally substituted with a fluorine or chlorine atom and/or all available hydrogen atoms are optionally substituted with deuterium. In some embodiments, R⁵ is selected from H, D, F, CH₃, CD₂H, CDH₂, CD₃, CF₃, CHF₂, CFH₂, CH₂CH₃, CH₂CH₂D, CH₂CD₂H and CD₂CD₃. In some embodiments, R⁵ is selected from H, D, F, CH₃, CD₂H, CDH₂, CD₃, CF₃, CHF₂, CF₂H, CH₂CH₃, CH₂CH₂D, CH₂CD₂H and CD₂CD₃. In some embodiments, R⁵ is selected from H, D, CH₃ and CD₃. In some embodiments, R⁵ is selected from H and D. In some embodiments, R⁵ is H. In some embodiments, R⁵ is selected from CH₃ and CD₃. In some embodiments, R⁵ is CD₃.

In some embodiments, R⁶, R⁷ and R⁸ are independently selected from H, halo, CN, OR¹², N(R¹²)(R¹³), SR¹², C₁-C₄alkyl, C₁-C₄haloalkyl, C₂-C₆haloalkenyl, CO₂R¹², C(O)N(R¹²)(R¹³), S(O)R¹², SO₂R¹², C₂-C₆alkenyl, C₂-C₆alkynyl, C₂-C₆haloalkynyl, C₃-C₇cycloalkyl and C₃-C₇heterocycloalkyl comprising 1 to 2 heteromoeities selected from O, S, S(O), SO₂, N and NR¹⁴, wherein said C₁-C₄alkyl, C₁-C₄haloalkyl, C₂-C₆alkenyl, C₂-C₆haloalkenyl, C₂-C₆alkynyl, C₂-C₆haloalkynyl, C₃-C₇cycloalkyl and C₃-C₇heterocycloalkyl are optionally substituted by one or more substituents independently selected from CN, OR¹⁵, N(R¹⁵)(R¹⁶) and SR¹⁵, and wherein said C₃-C₇cycloalkyl and C₃-C₇heterocycloalkyl are each further optionally substituted with one or more substituents selected from halo, CO₂R¹⁷, C(O)N(R¹⁷)(R¹⁸), SO₂R¹⁷, C₁-C₄alkyl, C₁-C₄haloalkyl, C₂-C₆alkenyl, C₂-C₆haloalkenyl, C₂-C₆alkynyl, C₂-C₆haloalkynyl, C₃-C₆cycloalkyl and C₃-C₆heterocycloalkyl comprising 1 to 2 heteromoeities selected from O, S, S(O), SO₂, N and NR¹⁹; wherein all available hydrogen atoms are optionally substituted with a fluorine or chlorine atom and/or all available atoms are optionally substituted with an alternate isotope thereof.

In some embodiments, R⁶, R⁷ and R⁸ are independently selected from H, halo, CN, OR¹², N(R¹²)(R¹³), SR¹², C₁-C₄alkyl, C₁-C₄haloalkyl, C₂-C₆haloalkenyl, CO₂R¹², C(O)N(R¹²)(R¹³), S(O)R¹², SO₂R¹², C₂-C₆alkenyl, C₂-C₆alkynyl and C₂-C₆haloalkynyl, wherein said C₁-C₄alkyl, C₁-C₄haloalkyl, C₂-C₆alkenyl, C₂-C₆haloalkenyl, C₂-C₆alkynyl and C₂-C₆haloalkynyl groups are optionally substituted by one or more substituents independently selected from CN, OR¹⁵, N(R¹⁵)(R¹⁶) and SR¹⁵, and wherein all available hydrogen atoms are optionally substituted with a fluorine or chlorine atom and/or all available atoms are optionally substituted with an alternate isotope thereof. In some embodiments, R⁶, R⁷ and R⁸ are independently selected from H, F, C, Br, CN, OR¹², N(R¹²)(R¹³), SR¹², C₁-C₄alkyl, C₁-C₄haloalkyl, C₂-C₆haloalkenyl, CO₂R¹², C(O)N(R¹²) (R¹³), S(O)R¹², SO₂R¹², C₂-C₆alkenyl, C₂-C₆alkynyl and C₂-C₆haloalkynyl, wherein said C₁-C₄alkyl, C₁-C₄haloalkyl, C₂-C₆alkenyl, C₂-C₆haloalkenyl, C₂-C₆alkynyl and C₂-C₆haloalkynyl groups are optionally substituted by one to three substituents independently selected from CN, OR¹⁵, N(R¹⁵)(R¹⁶) and SR¹⁵, wherein all available hydrogen atoms are optionally substituted with a fluorine or chlorine atom and/or all available atoms are optionally substituted with an alternate isotope thereof. In some embodiments, R⁶, R⁷ and R⁸ are independently selected from H, F, Cl, Br, CN, OR¹², N(R¹²)(R¹³), SR¹², CH₃, CH₂CH₃, CH(CH₃)₂, C(CH₃)₃, C₁-C₄haloalkyl, C₂-C₆haloalkenyl, CO₂R¹², S(O)R¹², SO₂R¹², C(O)N(R¹²)(R¹³), C₂-C₆alkenyl and C₂-C₆alkynyl, wherein said C₁-C₄alkyl, C₁-C₄haloalkyl, C₂-C₆alkenyl, C₂-C₆haloalkenyl and C₂-C₆alkynyl groups are optionally substituted by one or two substituents independently selected from CN, OR¹⁵, N(R¹⁵)(R¹⁵)₂ and SR¹⁵, wherein all available hydrogen atoms are optionally substituted with a fluorine or chlorine atom and/or all available atoms are optionally substituted with an alternate isotope thereof. In some embodiments, R⁶, R⁷ and R⁸ are independently selected from H, F, C, Br, CN, OR¹², N(R¹²)(R¹³), SR¹², CH₃, CH₂CH₃, CH(CH₃)₂, C(CH₃)₃, C₁-C₄haloalkyl, C₂-C₆haloalkenyl, CO₂R¹², S(O)R¹², SO₂R¹² and C₂-C₆alkenyl, wherein all available hydrogen atoms are optionally substituted with a fluorine or chlorine atom and/or all available atoms are optionally substituted with an alternate isotope thereof. In some embodiments, R⁶, R⁷ and R⁸ are independently selected from hydrogen, F, C₀, Br and CN. In some embodiments, R⁶, R⁷ and R⁸ are independently selected from H, D, F, C, Br and CN. In some embodiments, R⁶, R⁷ and R⁸ are independently selected from H and D. In some embodiments, R⁶, R⁷ and R⁸ are all H. In some embodiments, R⁶, R⁷ and R⁸ are all D. In some embodiments, R⁷ is selected from H, D, F, C, Br and CN and R⁶ and R⁸ are selected from hydrogen and deuterium. In some embodiments, R⁷ is selected from H, D, F and CN and R⁶ and R⁸ are selected from H and D. In some embodiments, R⁷ is selected from H, F and CN and R⁶ and R⁸ are selected from H and D. In some embodiments, R⁷ is selected from hydrogen, F and CN and R⁶ and R⁸ both H.

In some embodiments, the C₃-C₇cycloalkyl in R⁶, R⁷ and R⁸ is independently selected from cyclopropyl, cyclobutyl, cyclopentyl and cyclohexyl, wherein all available hydrogen atoms are optionally substituted with a fluorine or chlorine atom and/or all available atoms are optionally substituted with an alternate isotope thereof.

In some embodiments, the C₃-C₇heterocycloalkyl in R⁶, R⁷ and R⁸ is, independently, a saturated or unsaturated heterocycle. In some embodiments, the C₃-C₇heterocycloalkyl in R⁶, R⁷ and R⁸ is, independently, a saturated or unsaturated bridged bicyclic heterocycle. In some embodiments, the saturated or unsaturated bridged bicyclic heterocycle is independently selected from azabicyclohexanyl, diazabicycloheptanyl, oxobicyclohexanyl, oxobicycloheptanyl and oxobicycloheptanenyl, wherein all available hydrogen atoms are optionally substituted with a fluorine or chlorine atom and/or all available atoms are optionally substituted with an alternate isotope thereof.

In some embodiments, the C₃-C₇heterocycloalkyl in R⁶, R⁷ and R⁸ is independently selected from aziridinyl, oxiranyl, thiiranyl, oxaxiridinyl, dioxiranyl, azetidinyl, oxetanyl, theitanyl, diazetidinyl, dioxetanyl, dithietanyl, tetrahydrofuranyl, tetrahydrothiophenyl, pyrrolidinyl, imidazolidinyl, pyrazolidinyl, isoxthiolidinyl, thiazolidinyl, isothiazolidinyl, dioxolanyl, dithiolanyl, piperidinyl, triazolyl, furazanyl, oxadiazolyl, thiadiazolyl, dioxazolyl, dithiazolyl, tetrazolyl, oxatetrazolyl, tetrahydropyranyl, diazinanyl (e.g, piperazinyl), morpholinyl, thiomorpholinyl, dioxanyl, dithianyl, azepanyl, oxepanyl, thiepanyl and diazepanyl, wherein all available hydrogen atoms are optionally substituted with a fluorine or chlorine atom and/or all available atoms are optionally substituted with an alternate isotope thereof.

In some embodiments, Q is selected from P(O)OR⁹, C₁-C₂alkylene-P(O)OR⁹—C₁-C₂alkylene, C(O), SO₂, C(O)Q′C(O), C(O)OQ′OC(O) and C(O)NR^{9′}Q′NR^{9′}C(O), wherein all available hydrogen atoms are optionally substituted with a fluorine or chlorine atom and/or all available atoms are optionally substituted with an alternate isotope thereof. In some embodiments, Q is selected from direct bond, P(O)OR⁹, C₁-C₂alkylene-P(O)OR⁹—C₁-C₂alkylene, C(O), SO₂ and C(O)(Q′)C(O) wherein all available hydrogen atoms are optionally substituted with a fluorine or chlorine atom and/or all available atoms are optionally substituted with an alternate isotope thereof. In some embodiments, Q is selected from P(O)OR⁹ and C₁-C₂alkylene-P(O)OR⁹—C₁-C₂alkylene. In some embodiments, Q is C₁-C₂alkylene-P(O)OR⁹—C₁-C₂alkylene, wherein all available hydrogen atoms are optionally substituted with a fluorine or chlorine atom and/or all available atoms are optionally substituted with an alternate isotope thereof. In some embodiments, Q is CH₂—P(O)OR⁹—CH₂, wherein all available hydrogen atoms are optionally substituted with a fluorine or chlorine atom and/or all available atoms are optionally substituted with an alternate isotope thereof. In some embodiments, Q is selected from C(O), C(O)Q′C(O), C(O)OQ′OC(O) and C(O)NR^{9′}Q′NR^{9′}C(O), wherein all available hydrogen atoms are optionally substituted with a fluorine or chlorine atom and/or all available atoms are optionally substituted with an alternate isotope thereof. In some embodiments, Q is selected from a direct bond, C(O) and C(O)(Q′)C(O) wherein all available hydrogen atoms are optionally substituted with a fluorine or chlorine atom and/or all available atoms are optionally substituted with an alternate isotope thereof. In some embodiments, Q is a direct bond. In some embodiments, Q is C(O). In some embodiments, Q is C(O)(Q′)C(O). In some embodiments, Q is C(O)OQ′OC(O). In some embodiments, Q is C(O)NR^{9′}Q′NR^{9′}C(O). In some embodiments Q is SO₂.

In some embodiments, Q′ is selected from C₁-C₁₀alkylene, C₂-C₁₀alkenylene and C₂-C₁₀alkynylene wherein said C₁-C₁₀alkylene, C₂-C₁₀alkenylene and C₂-C₁₀alkynylene are optionally substituted by one to three substituents independently selected from CN, OR²¹, N(R²¹)(R²²), and SR²¹, and/or disubstituted on the same carbon atom with C₁₋₆alkyl, or with C₂₋₆alkylene to form a C₃-C₇cycloalkyl ring, wherein said C₃-C₇cycloalkyl is further optionally substituted with a substituent selected from C₁-C₃alkyl and C₁-C₃haloalkyl, wherein all available hydrogen atoms are optionally substituted with a fluorine or chlorine atom and/or all available atoms are optionally substituted with an alternate isotope thereof. In some embodiments, Q′ is selected from C₁-C₆alkylene, C₂-C₆alkenylene and C₂-C₆alkynylene optionally substituted with one or two substituents independently selected from OR²¹ and N(R²¹)(R²²), and/or disubstituted on the same carbon atom with C₁₋₆alkyl, or with C₂₋₆alkylene to form a C₃-C₇cycloalkyl ring, wherein said C₃-C₇cycloalkyl ring is further optionally substituted with a substituent selected from C₁-C₃alkyl and C₁-C₃haloalkyl, wherein all available hydrogen atoms are optionally substituted with a fluorine or chlorine atom and/or all available atoms are optionally substituted with an alternate isotope thereof. In some embodiments, Q′ is selected from C₁-C₄alkylene and C₂-C₄alkenylene, wherein all available hydrogen atoms are optionally substituted with a fluorine or chlorine atom and/or all available atoms are optionally substituted with an alternate isotope thereof. In some embodiments, Q′ is selected from CH₂, CH₂CH₂, CH₂CH₂CH₂ and CH═CH.

In some embodiments, when Q is C(O)Q′C(O), Q′ is a direct bond. In some embodiments, Q′ is a direct bond.

In some embodiments Q′ is selected from a C₃-C₇cycloalkylene and C₃-C₇heterocycloalkylene comprising 1 to 2 heteromoeities selected from O, S, S(O), SO₂, N, and NR²⁰, wherein said C₃-C₇cycloalkylene, and C₃-C₇heterocycloalkylene are optionally substituted with one to three substituents independently selected from CN, OR²¹, N(R²¹)(R²²), SR²¹, C₁-C₃alkyl and C₁-C₃haloalkyl. In some embodiments, the C₃-C₇cycloalkyl in Q′ selected from cyclopropylene, cyclobutylene, cyclopentylene and cyclohexylene, wherein all available hydrogen atoms are optionally substituted with a fluorine or chlorine atom and/or all available atoms are optionally substituted with an alternate isotope thereof.

In some embodiments, the C₃-C₇heterocycloalkyl in Q′ is selected from a saturated or unsaturated heterocycle. In some embodiments, the Q′ is selected from aziridinylene, oxiranylene, thiiranylene, oxaxiridinylene, dioxiranylene, azetidinylene, oxetanylene, theitanylene, diazetidinylene, dioxetanylene, dithietanylene, tetrahydrofuranylene, tetrahydrothiophenylene, pyrrolidinylene, imidazolidinylene, pyrazolidinylene, isoxthiolidinylene, thiazolidinylene, isothiazolidinylene, dioxolanylene, dithiolanylene, piperidinylene, triazolylene, furazanylene, oxadiazolylene, thiadiazolylene, dioxazolylene, dithiazolylene, tetrazolylene, oxatetrazolylene, tetrahydropyranylene, diazinanylene (e.g, piperazinylene), morpholinylene, thiomorpholinylene, dioxanylene, dithianylene, azepanylene, oxepanylene, thiepanylene and diazepanylene, wherein all available hydrogen atoms are optionally substituted with a fluorine or chlorine atom and/or all available atoms are optionally substituted with an alternate isotope thereof.

In some embodiments each R⁹ R¹⁰, R¹¹, R¹², R¹³, R¹⁴, R¹⁵, R¹⁶, R¹⁷, R¹⁸, R¹⁹, R²⁰, R²¹, and R²² is independently selected from H, substituted or unsubstituted C₁-C₄alkyl, substituted or unsubstituted C₂-C₆alkenyl, substituted or unsubstituted C₂-C₆alkynyl, substituted or unsubstituted C₁-C₄haloalkyl, substituted or unsubstituted C₃-C₇cycloalkyl, substituted or unsubstituted C₃-C₇heterocycloalkyl, substituted or unsubstituted aryl, substituted or unsubstituted heteroaryl, substituted or unsubstituted C₁-C₄alkyleneC₃-C₇cycloalkyl, substituted or unsubstituted C₁-C₄alkyleneC₃-C₇heterocycloalkyl, substituted or unsubstituted C₁-C₄alkylenearyl and substituted or unsubstituted C₁-C₄alkyleneheteroaryl, wherein all available hydrogen atoms are optionally substituted with a fluorine or chlorine atom and/or all available atoms are optionally substituted with an alternate isotope thereof.

In some embodiments each R⁹ R¹⁰, R¹¹, R¹², R¹³, R¹⁴, R¹⁵, R¹⁶, R¹⁷, R¹⁸, R¹⁹, R²⁰, R²¹, and R²² is independently selected from H, substituted or unsubstituted C₁-C₄alkyl, substituted or unsubstituted C₂-C₆alkenyl, substituted or unsubstituted C₂-C₆alkynyl, substituted or unsubstituted C₁-C₄haloalkyl, substituted or unsubstituted C₃-C₇cycloalkyl, substituted or unsubstituted heterocycloalkyl, substituted or unsubstituted aryl and substituted or unsubstituted heteroaryl.

In some embodiments, the C₃-C₇cycloalkyl in each R⁹, R¹⁰, R¹¹, R¹², R¹³, R¹⁴, R¹⁵, R¹⁶, R¹⁷, R¹⁸, R¹⁹, R²⁰, R²¹, and R²² is independently selected from cyclopropyl, cyclobutyl, cyclopentyl and cyclohexyl, wherein all available hydrogen atoms are optionally substituted with a fluorine or chlorine atom and/or all available atoms are optionally substituted with an alternate isotope thereof.

In some embodiments, the C₃-C₇heterocycloalkyl in each R⁹, R¹⁰, R¹¹, R¹², R¹³, R¹⁴, R¹⁵, R¹⁶, R¹⁷, R¹⁸, R¹⁹, R²⁰, R²¹, and R²² is independently selected from a saturated or unsaturated heterocycle. In some embodiments, the C₃-C₇heterocycloalkyl in each R⁹ R¹⁰, R¹¹, R¹², R¹³, R¹⁴, R¹⁵, R¹⁶, R¹⁷, R¹⁸, R¹⁹, R²⁰, R²¹, and R²² is independently selected from aziridinyl, oxiranyl, thiiranyl, oxaxiridinyl, dioxiranyl, azetidinyl, oxetanyl, theitanyl, diazetidinyl, dioxetanyl, dithietanyl, tetrahydrofuranyl, tetrahydrothiophenyl, pyrrolidinyl, imidazolidinyl, pyrazolidinyl, isoxthiolidinyl, thiazolidinyl, isothiazolidinyl, dioxolanyl, dithiolanyl, piperidinyl, triazolyl, furazanyl, oxadiazolyl, thiadiazolyl, dioxazolyl, dithiazolyl, tetrazolyl, oxatetrazolyl, tetrahydropyranyl, diazinanyl (e.g, piperazinyl), morpholinyl, thiomorpholinyl, dioxanyl, dithianyl, azepanyl, oxepanyl, thiepanyl and diazepanyl, wherein all available hydrogen atoms are optionally substituted with a fluorine or chlorine atom and/or all available atoms are optionally substituted with an alternate isotope thereof.

In some embodiments, the C₃-C₇heterocycloalkyl in R⁹ and R¹⁰ is independently selected from a saturated or unsaturated bridged bicyclic heterocycle. In some embodiments, the saturated or unsaturated bridged bicyclic heterocycle is independently selected from azabicyclohexanyl, diazabicycloheptanyl, oxobicyclohexanyl, oxobicycloheptanyl and oxobicycloheptanenyl, wherein all available hydrogen atoms are optionally substituted with a fluorine or chlorine atom and/or all available atoms are optionally substituted with an alternate isotope thereof.

In some embodiments, the heteroaryl in each R⁹, R¹⁰, R¹¹, R¹², R¹³, R¹⁴, R¹⁵, R¹⁶, R¹⁷, R¹⁸, R¹⁹, R²⁰, R²¹, and R²² is independently selected from azepinyl, benzisoxazolyl, benzofurazanyl, benzopyranyl, benzothiopyranyl, benzofuryl, benzothiazolyl, benzothienyl, benzoxazolyl, chromanyl, cinnolinyl, dihydrobenzofuryl, dihydrobenzothienyl, dihydrobenzothiopyranyl, dihydrobenzothiopyranyl sulfone, 1,3-dioxolanyl, furyl, imidazolidinyl, imidazolinyl, imidazolyl, indolinyl, indolyl, isochromanyl, isoindolinyl, isoquinolinyl, isothiazolidinyl, isothiazolyl, isothiazolidinyl, morpholinyl, naphthyridinyl, oxadiazolyl, 2-oxoazepinyl, oxazolyl, 2-oxopiperazinyl, 2-oxopiperdinyl, 2-oxopyrrolidinyl, piperidyl, piperazinyl, pyridyl, pyrazinyl, pyrazolidinyl, pyrazolyl, pyridazinyl, pyrimidinyl, pyrrolidinyl, pyrrolyl, quinazolinyl, quinolinyl, quinoxalinyl, tetrahydrofuryl, tetrahydroisoquinolinyl, tetrahydroquinolinyl, thiamorpholinyl, thiamorpholinyl sulfoxide, thiazolyl, thiazolinyl, thienofuryl, thienothienyl, triazolyl and thienyl, wherein all available hydrogen atoms are optionally substituted with a fluorine or chlorine atom and/or all available atoms are optionally substituted with an alternate isotope thereof.

In some embodiments, each R⁹, R¹⁰, R¹¹, R¹², R¹³, R¹⁴, R¹⁵, R¹⁶, R¹⁷, R¹⁸, R¹⁹, R²⁰, R²¹, and R²² is independently selected from H, substituted or unsubstituted C₁-C₄alkyl, substituted or unsubstituted C₂-C₆alkenyl, substituted or unsubstituted C₂-C₆alkynyl and substituted or unsubstituted C₁-C₄haloalkyl, wherein all available hydrogen atoms are optionally substituted with a fluorine or chlorine atom and/or all available atoms are optionally substituted with an alternate isotope thereof. In some embodiments, each R⁹, R¹⁰, R¹¹, R¹², R¹³, R¹⁴, R¹⁵, R¹⁶, R¹⁷, R¹⁸, R¹⁹, R²⁰, R²¹ and R²² is independently selected from H, C₁-C₄alkyl, C₂-C₆alkenyl, C₂-C₆alkynyl and C₁-C₄haloalkyl, wherein all available hydrogen atoms are optionally substituted with a fluorine or chlorine atom and/or all available atoms are optionally substituted with an alternate isotope thereof. In some embodiments, each R⁹, R¹⁰, R¹¹, R¹², R¹³, R¹⁴, R¹⁵, R¹⁶, R¹⁷, R¹⁸, R¹⁹, R²⁰, R²¹ and R²² is independently selected from H, C₁-C₄alkyl and C₂-C₆alkenyl, wherein all available hydrogen atoms are optionally substituted with a fluorine or chlorine atom and/or all available atoms are optionally substituted with an alternate isotope thereof. In some embodiments, each R⁹, R¹⁰, R¹¹, R¹², R¹³, R¹⁴, R¹⁵, R¹⁶, R¹⁷, R¹⁸, R¹⁹, R²⁰, R²¹ and R²² is independently selected from H and C₁-C₄alkyl, wherein all available hydrogen atoms are optionally and independently replaced with a fluorine atom or deuterium atom. In some embodiments, each R⁹, R¹⁰, R¹¹, R¹², R¹³, R¹⁴, R¹⁵, R¹⁶, R¹⁷, R¹⁸, R¹⁹, R²⁰, R²¹ and R²² is independently selected from H, D, CH₃, CD₂H, CDH₂, CD₃, CF₃, CHF₂, CF₂H, CH₂CH₂D, CH₂CD₂H, CH₂CH₃ and CD₂CD₃. In some embodiments, each R⁹, R¹⁰, R¹¹, R¹², R¹³, R¹⁴, R¹⁵, R¹⁶, R¹⁷, R¹⁸, R¹⁹, R²⁰, R²¹ and R²² is independently selected from H, D, CH₃ and CD₃.

In some embodiments, each R⁹, R¹⁰, R¹¹, R¹², R¹³, R¹⁴, R¹⁵, R¹⁶, R¹⁷, R¹⁸, R¹⁹, R²⁰, R²¹ and R²² is independently selected from substituted or unsubstituted C₁-C₄alkyleneC₃-C₇cycloalkyl, substituted or unsubstituted C₁-C₄alkyleneC₃-C₇heterocycloalkyl, substituted or unsubstituted C₁-C₄alkylenearyl, substituted or unsubstituted C₁-C₄alkyleneheteroaryl, wherein all available hydrogen atoms are optionally substituted with a fluorine or chlorine atom and/or all available atoms are optionally substituted with an alternate isotope thereof. In some embodiments, each R⁹, R¹⁰, R¹¹, R¹², R¹³, R¹⁴, R¹⁵, R¹⁶, R¹⁷, R¹⁸, R¹⁹, R²⁰, R²¹ and R²² is independently selected from substituted or unsubstituted C₁-C₄alkylenearyl and substituted or unsubstituted C₁-C₄alkyleneheteroaryl, wherein all available hydrogen atoms are optionally substituted with a fluorine or chlorine atom and/or all available atoms are optionally substituted with an alternate isotope thereof. In some embodiments, each R⁹, R¹⁰, R¹¹, R¹², R¹³, R¹⁴, R¹⁵, R¹⁶, R¹⁷, R¹⁸, R¹⁹, R²⁰, R²¹ and R²² is independently substituted or unsubstituted C₁-C₄alkylenearyl wherein all available hydrogen atoms are optionally substituted with a fluorine or chlorine atom and/or all available atoms are optionally substituted with an alternate isotope thereof. In some embodiments, each R⁹, R¹⁰, R¹¹, R¹², R¹³, R¹⁴, R¹⁵, R¹⁶, R¹⁷, R¹⁸, R¹⁹ and R²⁰ is independently substituted or unsubstituted CH₂aryl wherein all available hydrogen atoms are optionally substituted with a fluorine or chlorine atom and/or all available atoms are optionally substituted with an alternate isotope thereof. In some embodiments, each R⁹, R¹⁰, R¹¹, R¹², R¹³, R¹⁴, R¹⁵, R¹⁶, R¹⁷, R¹⁸, R¹⁹, R²⁰, R²¹, and R²² is independently substituted or unsubstituted CH₂phenyl.

When R⁹, R¹⁰, R¹¹, R¹², R¹³, R¹⁴, R¹⁵, R¹⁶, R¹⁷, R¹⁸, R¹⁹, R²⁰, R²¹, and R²² are substituted, in some embodiments, the substituents are independently selected from one or more of Br, C, F, CO₂H, CO₂CH₃, C(O)NH₂, C(O)N(CH₃)₂, C(O)NHCH₃, SO₂CH₃, C₁-C₄alkyl, C₁-C₄fluoralkyl, C₂-C₆alkenyl, C₂-C₆fluoroalkenyl, C₂-C₆alkynyl, C₂-C₆fluoroalkynyl, C₃-C₆cycloalkyl and a 3- to 6-membered heterocyclic ring including 1 to 2 ring heteromoeities selected from O, S, S(O), SO₂, N, NH and NCH₃. In some embodiments, the substituents on R⁹, R¹⁰, R¹¹, R¹², R¹³, R¹⁴, R¹⁵, R¹⁶, R¹⁷, R¹⁸, R¹⁹, R²⁰, R²¹, and R²²are independently selected from one to three of Br, Cl, F, C₁-C₄alkyl, C₁-C₄fluoralkyl, C₂-C₆alkenyl, C₂-C₆fluoroalkenyl, C₂-C₆alkynyl and C₂-C₆fluoroalkynyl. In some embodiments, the substituents on R⁹, R¹⁰, R¹¹, R¹², R¹³, R¹⁴, R¹⁵, R¹⁶, R¹⁷, R¹⁸, R¹⁹, R²⁰, R²¹, and R²² are independently selected from one or two of Br, Cl, F, CH₃, and CF₃.

In some embodiments, R^{9′} is selected from H and C₁-C₄alkyl, wherein all available hydrogen atoms are optionally substituted with a fluorine atom or chlorine atom and/or all available atoms are optionally substituted with an alternate isotope thereof. In some embodiments, R^{9′} is selected from H and C₁-C₄alkyl, wherein all available hydrogen atoms are optionally substituted with a fluorine atom or chlorine atom and/or all available hydrogen atoms are optionally substituted with deuterium. In some embodiments, R^{9′} is selected from hydrogen, CH₃, CH₂CH₃, CH(CH₃)₂ and C(CH₃)₃, wherein all available hydrogen atoms are optionally substituted with a fluorine or chlorine atom and/or all available hydrogen atoms are optionally substituted with deuterium. In some embodiments, R^{9′} is selected from H, D, F, CH₃, CD₂H, CDH₂, CD₃, CF₃, CHF₂, CFH₂, CH₂CH₃, CH₂CH₂D, CH₂CD₂H, CD₂CD₃, CD(CD₃)₂ and CH(CH₃)₂. In some embodiments, R^{9′} is selected from H, D, CH₃, CD₃ CH₂CH₃ and CH(CH₃)₂. In some embodiments, R^{9′} is selected from H, D, CH₃ and CD₃. In some embodiments, R^{9′} is selected from H and D. In some embodiments R^{9′} is selected from CH₃ and CD₃. In some embodiments, R^{9′} is CD₃. In some embodiments, R^{9′} is H.

In some embodiments, Q is P(O)(OH) and the compound of Formula I is a compound of Formula IA:

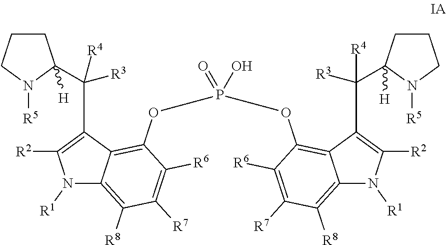

or a pharmaceutically acceptable salt, solvate and/or prodrug thereof,
wherein R¹, R², R³, R⁴, R⁵, R⁶, R⁷ and R⁸ are as defined in Formula I.

In some embodiments, Q is CH₂P(O)(OH)CH₂ and the compound of Formula I is a compound of Formula IB:

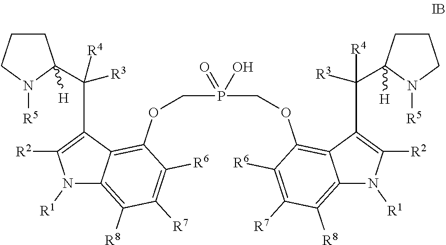

or a pharmaceutically acceptable salt, solvate and/or prodrug thereof,
wherein R¹, R², R³, R⁴, R⁵, R⁶, R⁷ and R⁸ are as defined in Formula I.

In some embodiments, Q is C(O)-Q′-C(O) and the compound of Formula I is a compound of Formula IC:

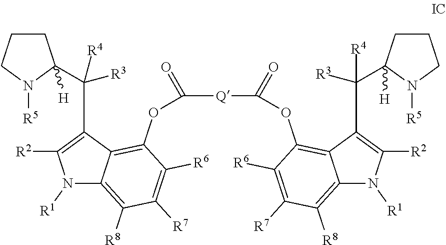

or a pharmaceutically acceptable salt, solvate and/or prodrug thereof,
wherein R¹, R², R³, R⁴, R⁵, R⁶, R⁷, R^{a} and Q′ are as defined in Formula I.

In some embodiments, Q is SO₂ and the compound of Formula I is a compound of Formula ID:

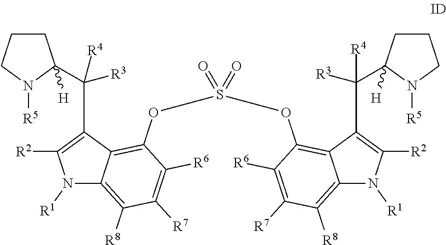

or a pharmaceutically acceptable salt, solvate and/or prodrug thereof,
wherein R¹, R², R³, R⁴, R⁵, R⁶, R⁷ and R⁸ are as defined in Formula I.

In some embodiments, Q is C(O)-Q′-C(O) and the compound of Formula I is a compound of Formula IE:

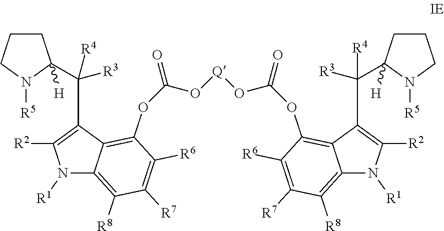

or a pharmaceutically acceptable salt, solvate and/or prodrug thereof,
wherein R¹, R², R³, R⁴, R⁵, R⁶, R⁷, R⁸ and Q′ are as defined in Formula I.

In some embodiments, Q is C(O)-Q′-C(O) and the compound of Formula I is a compound of Formula IF:

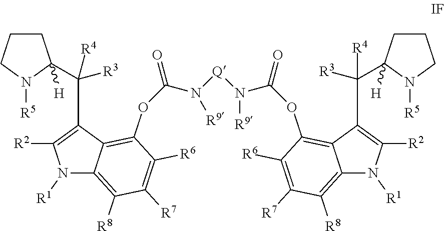

or a pharmaceutically acceptable salt, solvate and/or prodrug thereof,
wherein R¹, R², R³, R⁴, R⁵, R⁶, R⁷, R⁸, R⁹‘ and Q’ are as defined in Formula I.

In some embodiments, the compounds of Formula I are selected from the compounds listed in Table 1 below or a pharmaceutically acceptable salt, solvate and/or prodrug thereof

TABLE 1

| Compound ID | Chemical Structure |
|---|---|
| I-1 | 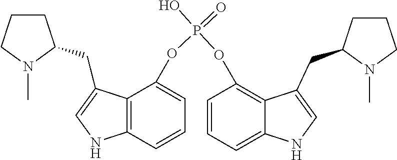 [SMILES: CN1CCC[C@@H]1Cc1c[nH]c2cccc(OP(=O)(O)Oc3cccc4[nH]cc(C[C@H]5CCCN5C)c34)c12] |
| I-2 | 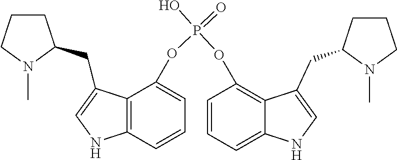 [SMILES: CN1CCC[C@H]1Cc1c[nH]c2cccc(OP(=O)(O)Oc3cccc4[nH]cc(C[C@@H]5CCCN5C)c34)c12] |
| I-3 | 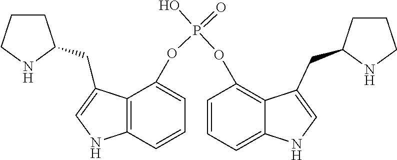 [SMILES: O=P(O)(Oc1cccc2[nH]cc(C[C@H]3CCCN3)c12)Oc1cccc2[nH]cc(C[C@H]3CCCN3)c12] |
| I-4 | 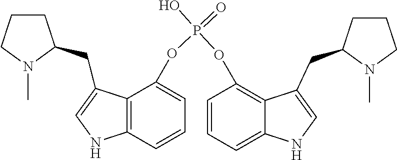 [SMILES: CN1CCC[C@@H]1Cc1c[nH]c2cccc(OP(=O)(O)Oc3cccc4[nH]cc(C[C@@H]5CCCN5C)c34)c12] |
| I-5 | 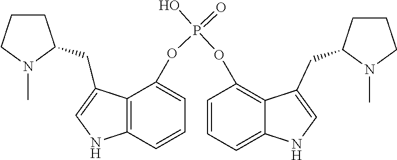 [SMILES: CN1CCC[C@@H]1Cc1c[nH]c2cccc(OP(=O)(O)Oc3cccc4[nH]cc(C[C@@H]5CCCN5C)c34)c12] |
| I-6 | 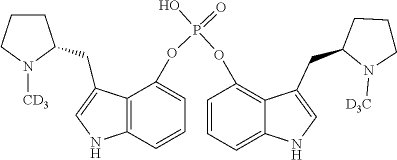 [SMILES: [2H]C([2H])([2H])N1CCC[C@@H]1Cc1c[nH]c2cccc(OP(=O)(O)Oc3cccc4[nH]cc(C[C@H]5CCCN5C([2H])([2H])[2H])c34)c12] |
| I-7 | 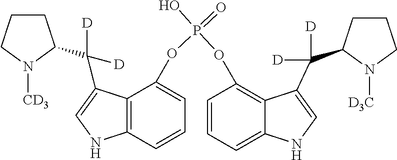 [SMILES: [2H]C([2H])(c1c[nH]c2cccc(OP(=O)(O)Oc3cccc4[nH]cc(C([2H])([2H])[C@H]5CCCN5C([2H])([2H])[2H])c34)c12)[C@H]1CCCN1C([2H])([2H])[2H]] |
| I-8 | 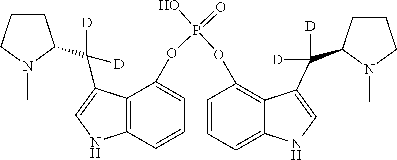 [SMILES: [2H]C([2H])(c1c[nH]c2cccc(OP(=O)(O)Oc3cccc4[nH]cc(C([2H])([2H])[C@H]5CCCN5C)c34)c12)[C@H]1CCCN1C] |
| I-9 | 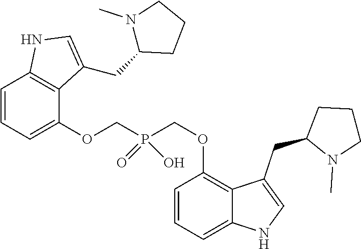 [SMILES: CN1CCC[C@@H]1Cc1c[nH]c2cccc(OCP(=O)(O)COc3cccc4[nH]cc(C[C@H]5CCCN5C)c34)c12] |
| I-10 | 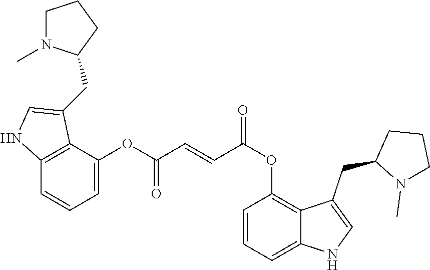 [SMILES: CN1CCC[C@@H]1Cc1c[nH]c2cccc(OC(=O)/C=C/C(=O)Oc3cccc4[nH]cc(C[C@H]5CCCN5C)c34)c12] |
| I-11 | 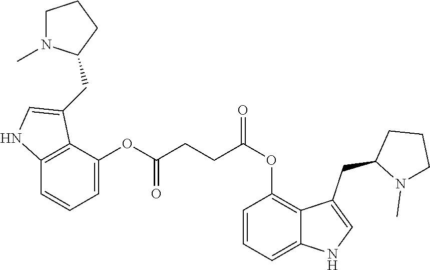 [SMILES: CN1CCC[C@@H]1Cc1c[nH]c2cccc(OC(=O)CCC(=O)Oc3cccc4[nH]cc(C[C@H]5CCCN5C)c34)c12] |
| I-12 | 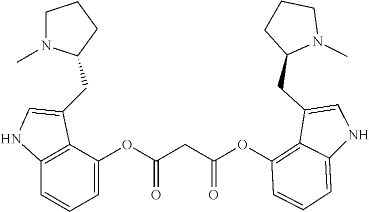 [SMILES: CN1CCC[C@@H]1Cc1c[nH]c2cccc(OC(=O)CC(=O)Oc3cccc4[nH]cc(C[C@H]5CCCN5C)c34)c12] |
| I-13 | 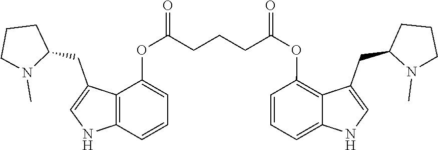 [SMILES: CN1CCC[C@@H]1Cc1c[nH]c2cccc(OC(=O)CCCC(=O)Oc3cccc4[nH]cc(C[C@H]5CCCN5C)c34)c12] |
| I-14 | 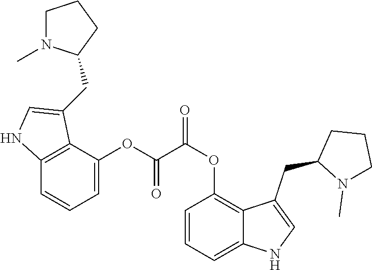 [SMILES: CN1CCC[C@@H]1Cc1c[nH]c2cccc(OC(=O)C(=O)Oc3cccc4[nH]cc(C[C@H]5CCCN5C)c34)c12] |
| I-15 | 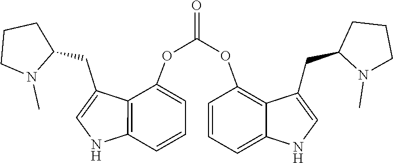 [SMILES: CN1CCC[C@@H]1Cc1c[nH]c2cccc(OC(=O)Oc3cccc4[nH]cc(C[C@H]5CCCN5C)c34)c12] |
| I-16 | 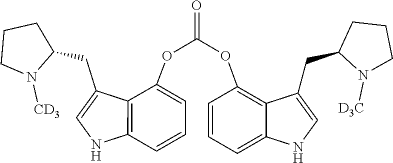 [SMILES: [2H]C([2H])([2H])N1CCC[C@@H]1Cc1c[nH]c2cccc(OC(=O)Oc3cccc4[nH]cc(C[C@H]5CCCN5C([2H])([2H])[2H])c34)c12] |
| I-17 | 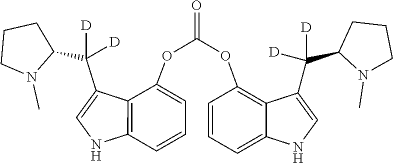 [SMILES: [2H]C([2H])(c1c[nH]c2cccc(OC(=O)Oc3cccc4[nH]cc(C([2H])([2H])[C@H]5CCCN5C)c34)c12)[C@H]1CCCN1C] |
| I-18 | 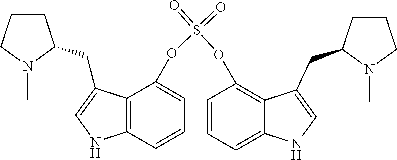 [SMILES: CN1CCC[C@@H]1Cc1c[nH]c2cccc(OS(=O)(=O)Oc3cccc4[nH]cc(C[C@H]5CCCN5C)c34)c12] |
| I-19 | 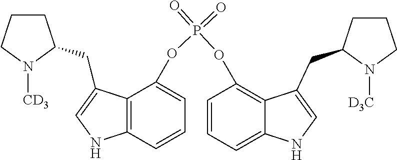 |
| I-20 | 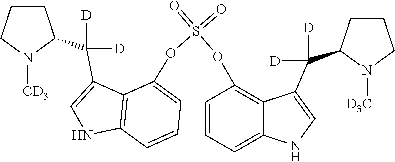 [SMILES: [2H]C([2H])(c1c[nH]c2cccc(OS(=O)(=O)Oc3cccc4[nH]cc(C([2H])([2H])[C@H]5CCCN5C([2H])([2H])[2H])c34)c12)[C@H]1CCCN1C([2H])([2H])[2H]] |
| I-21 | 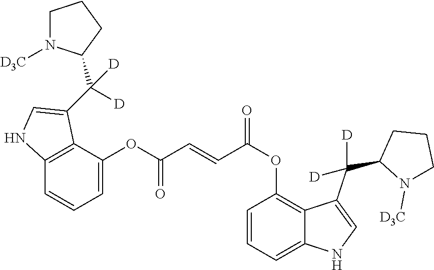 [SMILES: [2H]C([2H])(c1c[nH]c2cccc(OC(=O)/C=C/C(=O)Oc3cccc4[nH]cc(C([2H])([2H])[C@H]5CCCN5C([2H])([2H])[2H])c34)c12)[C@H]1CCCN1C([2H])([2H])[2H]] |
| I-22 | 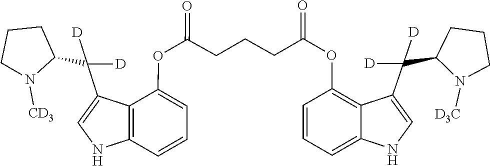 [SMILES: [2H]C([2H])(c1c[nH]c2cccc(OC(=O)CCCC(=O)Oc3cccc4[nH]cc(C([2H])([2H])[C@H]5CCCN5C([2H])([2H])[2H])c34)c12)[C@H]1CCCN1C([2H])([2H])[2H]] |
| I-23 | 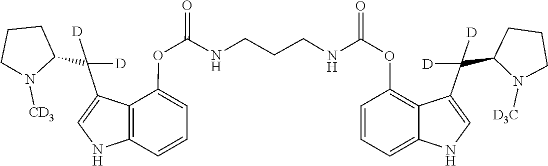 [SMILES: [2H]C([2H])(c1c[nH]c2cccc(OC(=O)NCCCNC(=O)Oc3cccc4[nH]cc(C([2H])([2H])[C@H]5CCCN5C([2H])([2H])[2H])c34)c12)[C@H]1CCCN1C([2H])([2H])[2H]] |
| I-24 | 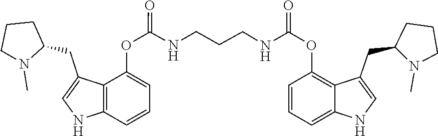 [SMILES: CN1CCC[C@@H]1Cc1c[nH]c2cccc(OC(=O)NCCCNC(=O)Oc3cccc4[nH]cc(C[C@H]5CCCN5C)c34)c12] |
| I-25 | 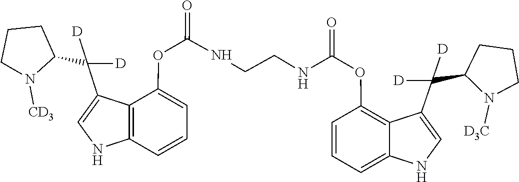 [SMILES: [2H]C([2H])(c1c[nH]c2cccc(OC(=O)NCCNC(=O)Oc3cccc4[nH]cc(C([2H])([2H])[C@H]5CCCN5C([2H])([2H])[2H])c34)c12)[C@H]1CCCN1C([2H])([2H])[2H]] |
| I-26 | 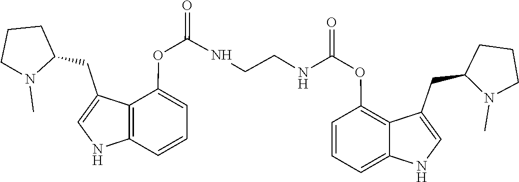 [SMILES: CN1CCC[C@@H]1Cc1c[nH]c2cccc(OC(=O)NCCNC(=O)Oc3cccc4[nH]cc(C[C@H]5CCCN5C)c34)c12] |
| I-27 | 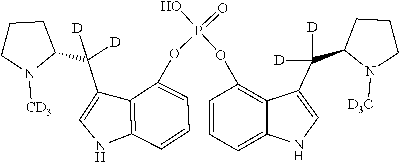 [SMILES: [2H]C([2H])(c1c[nH]c2cccc(OP(=O)(O)Oc3cccc4[nH]cc(C([2H])([2H])[C@H]5CCCN5C([2H])([2H])[2H])c34)c12)[C@H]1CCCN1C([2H])([2H])[2H]] |
| I-28 | 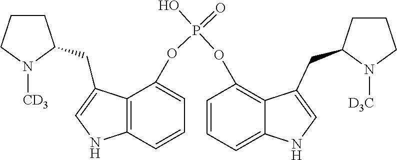 [SMILES: [2H]C([2H])([2H])N1CCC[C@@H]1Cc1c[nH]c2cccc(OP(=O)(O)Oc3cccc4[nH]cc(C[C@H]5CCCN5C([2H])([2H])[2H])c34)c12] |
| I-29 | 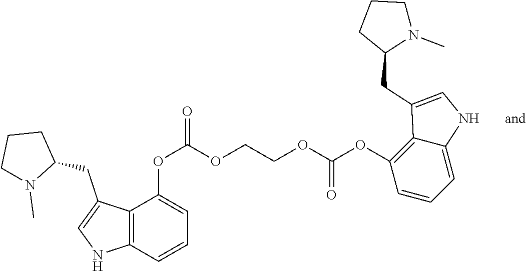 [SMILES: CN1CCC[C@@H]1Cc1c[nH]c2cccc(OC(=O)OCCOC(=O)Oc3cccc4[nH]cc(C[C@H]5CCCN5C)c34)c12 ; "and"] |
| I-30 | 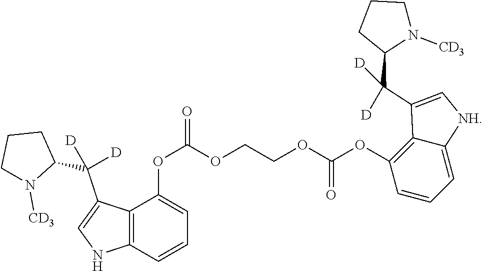 [SMILES: [2H]C([2H])(c1c[nH]c2cccc(OC(=O)OCCOC(=O)Oc3cccc4[nH]cc(C([2H])([2H])[C@H]5CCCN5C([2H])([2H])[2H])c34)c12)[C@H]1CCCN1C([2H])([2H])[2H]] |

In some embodiments, the compounds of Formula I are selected from one or more of the compounds listed in Table 1 or a pharmaceutically acceptable salt, solvate and/or prodrug thereof. In some embodiments, the compounds of Formula I are selected from the compounds listed in Table 1 or a pharmaceutically acceptable salt, solvate and/or prodrug thereof and a combination thereof.

In some embodiments, the compounds of Formula I breakdown in vivo to provide active metabolites. Therefore, in some embodiments, the present application includes a compound of Formula I and a metabolite thereof. In some embodiments, the present application includes a compound of Formula I or pharmaceutically acceptable salt, solvate, metabolite and/or prodrug thereof.

In some embodiments, the active metabolite is a compound of Formula II:

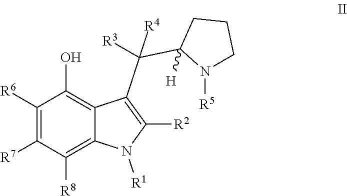

wherein R¹, R², R³, R⁴, R⁵, R⁶, R⁷ and R⁸ are defined above for Formula I, including embodiments thereof.

In some embodiments, the pharmaceutically acceptable salt is an acid addition salt or a base addition salt. The selection of a suitable salt may be made by a person skilled in the art. Suitable salts include acid addition salts that may, for example, be formed by mixing a solution of a compound with a solution of a pharmaceutically acceptable acid such as hydrochloric acid, sulfuric acid, acetic acid, trifluoroacetic acid, or benzoic acid. Additionally, acids that are generally considered suitable for the formation of pharmaceutically useful salts from basic pharmaceutical compounds are discussed, for example, by P. Stahl et al, Camille G. (eds.) and Handbook of Pharmaceutical Salts. Properties, Selection and Use. (2002) Zurich: Wiley VCH; S. Berge et al, Journal of Pharmaceutical Sciences 1977 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website).

An acid addition salt suitable for, or compatible with, the treatment of subjects is any non-toxic organic or inorganic acid addition salt of any basic compound. Basic compounds that form an acid addition salt include, for example, compounds comprising an amine group. Illustrative inorganic acids which form suitable salts include hydrochloric, hydrobromic, sulfuric, nitric and phosphoric acids, as well as acidic metal salts such as sodium monohydrogen orthophosphate and potassium hydrogen sulfate. Illustrative organic acids which form suitable salts include mono-, di- and tricarboxylic acids. Illustrative of such organic acids are, for example, acetic, trifluoroacetic, propionic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, mandelic, salicylic, 2-phenoxybenzoic, p-toluenesulfonic acid and other sulfonic acids such as methanesulfonic acid, ethanesulfonic acid and 2-hydroxyethanesulfonic acid. In some embodiments, exemplary acid addition salts also include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates (“mesylates”), naphthalenesulfonates, nitrates, oxalates, phosphates, propionates, salicylates, succinates, sulfates, tartarates, thiocyanates, toluenesulfonates (also known as tosylates) and the like. In some embodiments, the mono- or di-acid salts are formed and such salts exist in either a hydrated, solvated or substantially anhydrous form. In general, acid addition salts are more soluble in water and various hydrophilic organic solvents and generally demonstrate higher melting points in comparison to their free base forms. The selection criteria for the appropriate salt will be known to one skilled in the art. Other non-pharmaceutically acceptable salts such as but not limited to oxalates may be used, for example in the isolation of compounds of the application for laboratory use, or for subsequent conversion to a pharmaceutically acceptable acid addition salt.

A base addition salt suitable for, or compatible with, the treatment of subjects is any non-toxic organic or inorganic base addition salt of any acidic compound. Acidic compounds that form a basic addition salt include, for example, compounds comprising a carboxylic acid group. Illustrative inorganic bases which form suitable salts include lithium, sodium, potassium, calcium, magnesium or barium hydroxide as well as ammonia. Illustrative organic bases which form suitable salts include aliphatic, alicyclic or aromatic organic amines such as isopropylamine, methylamine, trimethylamine, picoline, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Exemplary organic bases are isopropylamine, diethylamine, ethanolamine, trimethylamine, dicyclohexylamine, choline and caffeine. The selection of the appropriate salt may be useful, for example, so that an ester functionality, if any, elsewhere in a compound is not hydrolyzed. The selection criteria for the appropriate salt will be known to one skilled in the art. In some embodiments, exemplary basic salts also include ammonium salts, alkali metal salts such as sodium, lithium and potassium salts, alkaline earth metal salts such as calcium and magnesium salts, salts with organic bases (for example, organic amines) such as dicyclohexylamine, Abutyl amine, choline and salts with amino acids such as arginine, lysine and the like. Basic nitrogen containing groups may be quarternized with agents such as lower alkyl halides (e.g., methyl, ethyl and butyl chlorides, bromides and iodides), dialkyl sulfates (e.g., dimethyl, diethyl and dibutyl sulfates), long chain halides (e.g., decyl, lauryl and stearyl chlorides, bromides and iodides), aralkyl halides (e.g., benzyl and phenethyl bromides) and others. Compounds carrying an acidic moiety can be mixed with suitable pharmaceutically acceptable salts to provide, for example, alkali metal salts (e.g., sodium or potassium salts), alkaline earth metal salts (e.g., calcium or magnesium salts) and salts formed with suitable organic ligands such as quaternary ammonium salts. Also, in the case of an acid (—COOH) or alcohol group being present, pharmaceutically acceptable esters can be employed to modify the solubility or hydrolysis characteristics of the compound.

All such acid salts and base salts are intended to be pharmaceutically acceptable salts within the scope of the application and all acid and base salts are considered equivalent to the free forms of the corresponding compounds for purposes of the application. In addition, when a compound of the application contains both a basic moiety, such as, but not limited to an aliphatic primary, secondary, tertiary or cyclic amine, an aromatic or heteroaryl amine, pyridine or imidazole and an acidic moiety, such as, but not limited to tetrazole or carboxylic acid, zwitterions (“inner salts”) may be formed and are included within the terms “salt(s)” as used herein. It is understood that certain compounds of the application may exist in zwitterionic form, having both anionic and cationic centers within the same compound and a net neutral charge. Such zwitterions are included within the application.

Solvates of compounds of the application include, for example, those made with solvents that are pharmaceutically acceptable. Examples of such solvents include water (resulting solvate is called a hydrate) and ethanol and the like. Suitable solvents are physiologically tolerable at the dosage administered.

Prodrugs of the compounds of the present application include, for example, conventional esters formed with available hydroxy, thiol, amino or carboxyl groups. Some common esters which have been utilized as prodrugs are phenyl esters, aliphatic (C₁-C₂₄) esters, acyloxymethyl esters, carbamates and amino acid esters.

It is understood and appreciated that in some embodiments, compounds of the present application may have at least one chiral center and therefore can exist as enantiomers and/or diastereomers. It is to be understood that all such isomers and mixtures thereof in any proportion are encompassed within the scope of the present application. It is to be further understood that while the stereochemistry of the compounds may be as shown in any given compound listed herein, such compounds may also contain certain amounts (for example, less than 20%, suitably less than 10%, more suitably less than 5%) of compounds of the present application having an alternate stereochemistry. It is intended that any optical isomers, as separated, pure or partially purified optical isomers or racemic mixtures thereof are included within the scope of the present application.

In some embodiments, the compounds of the present application can also include tautomeric forms, such as keto-enol tautomers and the like. Tautomeric forms can be in equilibrium or sterically locked into one form by appropriate substitution. It is intended that any tautomeric forms which the compounds form, as well as mixtures thereof, are included within the scope of the present application.

The compounds of the present application may further exist in varying amorphous and polymorphic forms and it is contemplated that any amorphous forms, polymorphs, or mixtures thereof, which form are included within the scope of the present application.

The compounds of the present application may further be radiolabeled and accordingly all radiolabeled versions of the compounds of the application are included within the scope of the present application, the compounds of the application also include those in which one or more radioactive atoms are incorporated within their structure.

### III. Compositions

The compounds of the present application are suitably formulated in a conventional manner into compositions using one or more carriers. Accordingly, the present application also includes a composition comprising one or more compounds of the application and a carrier. The compounds of the application are suitably formulated into pharmaceutical compositions for administration to subjects in a biologically compatible form suitable for administration in vivo. Accordingly, the present application further includes a pharmaceutical composition comprising one or more compounds of the application and a pharmaceutically acceptable carrier. In embodiments of the application, the pharmaceutical compositions are used in the treatment of any of the diseases, disorders or conditions described herein.

The compounds of the application are administered to a subject in a variety of forms depending on the selected route of administration, as will be understood by those skilled in the art. For example, a compound of the application is administered by oral, inhalation, parenteral, buccal, sublingual, insufflation, epidurally, nasal, rectal, vaginal, patch, pump, minipump, topical or transdermal administration and the pharmaceutical compositions formulated accordingly. In some embodiments, administration is by means of a pump for periodic or continuous delivery. Conventional procedures and ingredients for the selection and preparation of suitable compositions are described, for example, in Remington's Pharmaceutical Sciences (2000-20^{th} edition) and in The United States Pharmacopeia: The National Formulary (USP 24 NF19) published in 1999.

Parenteral administration includes systemic delivery routes other than the gastrointestinal (GI) tract and includes, for example intravenous, intra-arterial, intraperitoneal, subcutaneous, intramuscular, transepithelial, nasal, intrapulmonary (for example, by use of an aerosol), intrathecal, rectal and topical (including the use of a patch or other transdermal delivery device) modes of administration. Parenteral administration may be by continuous infusion over a selected period of time.

In some embodiments, a compound of the application is orally administered, for example, with an inert diluent or with an assimilable edible carrier, or it is enclosed in hard or soft shell gelatin capsules, or it is compressed into tablets, or it is incorporated directly with the food of the diet. In some embodiments, the compound is incorporated with excipient and used in the form of ingestible tablets, buccal tablets, troches, capsules, caplets, pellets, granules, lozenges, chewing gum, powders, syrups, elixirs, wafers, aqueous solutions and suspensions and the like. In the case of tablets, carriers that are used include lactose, corn starch, sodium citrate and salts of phosphoric acid. Pharmaceutically acceptable excipients include binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate), or solvents (e.g. medium chain triglycerides, ethanol, water). In embodiments, the tablets are coated by methods well known in the art. In the case of tablets, capsules, caplets, pellets or granules for oral administration, pH sensitive enteric coatings, such as Eudragits™ designed to control the release of active ingredients are optionally used. Oral dosage forms also include modified release, for example immediate release and timed-release, formulations. Examples of modified-release formulations include, for example, sustained-release (SR), extended-release (ER, XR, or XL), time-release or timed-release, controlled-release (CR), or continuous-release (CR or Contin), employed, for example, in the form of a coated tablet, an osmotic delivery device, a coated capsule, a microencapsulated microsphere, an agglomerated particle, e.g., as of molecular sieving type particles, or, a fine hollow permeable fiber bundle, or chopped hollow permeable fibers, agglomerated or held in a fibrous packet. Timed-release compositions are formulated, for example as liposomes or those wherein the active compound is protected with differentially degradable coatings, such as by microencapsulation, multiple coatings, etc. Liposome delivery systems include, for example, small unilamellar vesicles, large unilamellar vesicles and multilamellar vesicles. In some embodiments, liposomes are formed from a variety of phospholipids, such as cholesterol, stearylamine or phosphatidylcholines. For oral administration in a capsule form, useful carriers, solvents or diluents include lactose, medium chain triglycerides, ethanol and dried corn starch.

In some embodiments, liquid preparations for oral administration take the form of, for example, solutions, syrups or suspensions, or they are suitably presented as a dry product for constitution with water or other suitable vehicle before use. When aqueous suspensions and/or emulsions are administered orally, the compound of the application is suitably suspended or dissolved in an oily phase that is combined with emulsifying and/or suspending agents. If desired, certain sweetening and/or flavoring and/or coloring agents are added. Such liquid preparations for oral administration are prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., medium chain triglycerides, almond oil, oily esters or ethyl alcohol); and preservatives (e.g., methyl or propyl p-hydroxybenzoates or sorbic acid). Useful diluents include lactose and high molecular weight polyethylene glycols.

It is also possible to freeze-dry the compounds of the application and use the lyophilizates obtained, for example, for the preparation of products for injection.

In some embodiments, a compound of the application is administered parenterally. For example, solutions of a compound of the application are prepared in water suitably mixed with a surfactant such as hydroxypropylcellulose. In some embodiments, dispersions are prepared in glycerol, liquid polyethylene glycols, DMSO and mixtures thereof with or without alcohol and in oils. Under ordinary conditions of storage and use, these preparations contain a preservative to prevent the growth of microorganisms. A person skilled in the art would know how to prepare suitable formulations. For parenteral administration, sterile solutions of the compounds of the application are usually prepared and the pH's of the solutions are suitably adjusted and buffered. For intravenous use, the total concentration of solutes should be controlled to render the preparation isotonic. For ocular administration, ointments or droppable liquids are delivered, for example, by ocular delivery systems known to the art such as applicators or eye droppers. In some embodiments, such compositions include mucomimetics such as hyaluronic acid, chondroitin sulfate, hydroxypropyl methylcellulose or polyvinyl alcohol, preservatives such as sorbic acid, EDTA or benzyl chromium chloride and the usual quantities of diluents or carriers. For pulmonary administration, diluents or carriers will be selected to be appropriate to allow the formation of an aerosol.

In some embodiments, a compound of the application is formulated for parenteral administration by injection, including using conventional catheterization techniques or infusion. Formulations for injection are, for example, presented in unit dosage form, e.g., in ampoules or in multi-dose containers, with an added preservative. In some embodiments, the compositions take such forms as sterile suspensions, solutions or emulsions in oily or aqueous vehicles and contain formulating agents such as suspending, stabilizing and/or dispersing agents. In all cases, the form must be sterile and must be fluid to the extent that easy syringability exists. Alternatively, the compounds of the application are suitably in a sterile powder form for reconstitution with a suitable vehicle, e.g., sterile pyrogen-free water, before use.

In some embodiments, compositions for nasal administration are conveniently formulated as aerosols, drops, gels and powders. For intranasal administration or administration by inhalation, the compounds of the application are conveniently delivered in the form of a solution, dry powder formulation or suspension from a pump spray container that is squeezed or pumped by the patient or as an aerosol spray presentation from a pressurized container or a nebulizer. Aerosol formulations typically comprise a solution or fine suspension of the active substance in a physiologically acceptable aqueous or non-aqueous solvent and are usually presented in single or multidose quantities in sterile form in a sealed container, which, for example, take the form of a cartridge or refill for use with an atomising device. Alternatively, the sealed container is a unitary dispensing device such as a single dose nasal inhaler or an aerosol dispenser fitted with a metering valve which is intended for disposal after use. Where the dosage form comprises an aerosol dispenser, it will contain a propellant which is, for example, a compressed gas such as compressed air or an organic propellant such as fluorochlorohydrocarbon. Suitable propellants include but are not limited to dichlorodifluoromethane, trichlorofluoromethane, dichlorotetrafluoroethane, heptafluoroalkanes, carbon dioxide or another suitable gas. In the case of a pressurized aerosol, the dosage unit is suitably determined by providing a valve to deliver a metered amount. In some embodiments, the pressurized container or nebulizer contains a solution or suspension of the active compound. Capsules and cartridges (made, for example, from gelatin) for use in an inhaler or insufflator are, for example, formulated containing a powder mix of a compound of the application and a suitable powder base such as lactose or starch. The aerosol dosage forms can also take the form of a pump-atomizer.

Compositions suitable for buccal or sublingual administration include tablets, lozenges and pastilles, wherein a compound of the application is formulated with a carrier such as sugar, acacia, tragacanth, or gelatin and glycerine. Compositions for rectal administration are conveniently in the form of suppositories containing a conventional suppository base such as cocoa butter.

Suppository forms of the compounds of the application are useful for vaginal, urethral and rectal administrations. Such suppositories will generally be constructed of a mixture of substances that is solid at room temperature but melts at body temperature. The substances commonly used to create such vehicles include but are not limited to theobroma oil (also known as cocoa butter), glycerinated gelatin, other glycerides, hydrogenated vegetable oils, mixtures of polyethylene glycols of various molecular weights and fatty acid esters of polyethylene glycol. See, for example: Remington's Pharmaceutical Sciences, 16^{th} Ed., Mack Publishing, Easton, PA, 1980, pp. 1530-1533 for further discussion of suppository dosage forms.

In some embodiments a compound of the application is coupled with soluble polymers as targetable drug carriers. Such polymers include, for example, polyvinylpyrrolidone, pyran copolymer, polyhydroxypropylmethacrylamide-phenol, polyhydroxy-ethylaspartamide-phenol, or polyethyleneoxide-polylysine substituted with palmitoyl residues. Furthermore, in some embodiments, a compound of the application is coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and crosslinked or amphipathic block copolymers of hydrogels.

The compounds of the application are particularly amenable to administration with the air of nano-carrier systems, such as liposomes, micelles, nanoparticles, nano-emulsions, lipidic nano-systems and the like (see for example, Bhat, M. et al. Chem. And Phys. Of Lipids, 2021, 236, 105053). Accordingly the present application includes a composition comprising one or more compounds of the application and one or more components of a nano-carrier system.

A compound of the application including pharmaceutically acceptable salts and/or solvates thereof is suitably used on their own but will generally be administered in the form of a pharmaceutical composition in which the one or more compounds of the application (the active ingredient) is in association with a pharmaceutically acceptable carrier. Depending on the mode of administration, the pharmaceutical composition will comprise from about 0.05 wt % to about 99 wt % or about 0.10 wt % to about 70 wt %, of the active ingredient and from about 1 wt % to about 99.95 wt % or about 30 wt % to about 99.90 wt % of a pharmaceutically acceptable carrier, all percentages by weight being based on the total composition.

In some embodiments, the compounds of the application including pharmaceutically acceptable salts and/or solvates thereof are used are administered in a composition comprising an additional therapeutic agent. Therefore the present application also includes a pharmaceutical composition comprising one of more compounds of the application, or pharmaceutically acceptable salts and/or solvates thereof and an additional therapeutic agent, and optionally one or more pharmaceutically acceptable excipients. In some embodiments, the additional therapeutic agent is another known agent useful for treatment of a disease, disorder or condition by activation of a serotonin receptor, for example those listed in the Methods and Uses section below. In some embodiments, the additional therapeutic agent is a psychoactive drug.

In the above, the term “a compound” also includes embodiments wherein one or more compounds are referenced.

### IV. Methods and Uses of the Application

Compounds of the application are useful for treating diseases, disorders or conditions by activating a serotonin receptor. Therefore, the compounds of the present application are useful as medicaments. Accordingly, the application also includes a compound of the application for use as a medicament.

The present application also includes a method of treating a disease, disorder or condition that is treated by activation of a serotonin receptor comprising administering a therapeutically effective amount of one or more compounds of the application to a subject in need thereof, i.e. a subject having the disease, disorder or condition.

The present application also includes a use of one or more compounds of the application for treatment of a disease, disorder or condition that is treated by activation of a serotonin receptor as well as a use of one or more compounds of the application for the preparation of a medicament for treatment of a disease, disorder or condition that is treated by activation of a serotonin receptor. The application further includes one or more compounds of the application for use in treating a disease, disorder or condition that is treated by activation of a serotonin receptor.

In some embodiments, the serotonin receptor is 5-HT_{2A}. Accordingly, the present application includes a method for activating 5-HT_{2A} in a cell, either in a biological sample or in a patient, comprising administering an effective amount of one or more compounds of the application to the cell. The application also includes a use of one or more compounds of the application for activating 5-HT_{2A} in a cell as well as a use of one or more compounds of the application for the preparation of a medicament for activating 5-HT_{2A} in a cell. The application further includes one or more compounds of the application for use in activating 5-HT_{2A} in a cell. In some embodiments, the method for activating 5-HT_{2A} is in or on a cell.

The present application also includes a method of treating a disease, disorder or condition that is treated by activation of 5-HT_{2A} comprising administering a therapeutically effective amount of one or more compounds of the application to a subject in need thereof, that is a subject having the disease, disorder or condition. The present application also includes a use of one or more compounds of the application for treatment of a disease, disorder or condition that is treated by activation of 5-HT_{2A} as well as a use of one or more compounds of the application for the preparation of a medicament for treatment of a disease, disorder or condition that is treated by activation of 5-HT_{2A}. The application further includes one or more compounds of the application for use in treating a disease, disorder or condition that is treated by activation of 5-HT_{2A}.

In some embodiments, the compounds of the application are useful for preventing, treating and/or reducing the severity of a mental illness disorder and/or condition that is treated by activation of 5-HT_{2A} in a subject. Therefore, in some embodiments, the disease, disorder or condition that is treated by activation of a serotonin receptor is a mental illness. Accordingly, the present application also includes a method of treating a mental illness comprising administering a therapeutically effective amount of one or more compounds of the application to a subject in need thereof. The present application also includes a use of one or more compounds of the application for treatment of a mental illness, as well as a use of one or more compounds of the application for the preparation of a medicament for treatment of a mental illness. The application further includes one or more compounds of the application for use in treating a mental illness.

In some embodiments, the mental illness is selected from anxiety disorders such as generalized anxiety disorder, panic disorder, social anxiety disorder and specific phobias; depression such as, hopelessness, loss of pleasure, fatigue and suicidal thoughts; mood disorders, such as depression, bipolar disorder, cancer-related depression, anxiety and cyclothymic disorder; psychotic disorders, such as hallucinations, delusions, schizophrenia; impulse control and addiction disorders, such as pyromania (starting fires), kleptomania (stealing) and compulsive gambling; alcohol addiction; drug addiction, such as opioid addiction; personality disorders, such as antisocial personality disorder, obsessive-compulsive personality disorder and paranoid personality disorder; obsessive-compulsive disorder (OCD), such as thoughts or fears that cause a subject to perform certain rituals or routines; post-traumatic stress disorder (PTSD); stress response syndromes (formerly called adjustment disorders); dissociative disorders, formerly called multiple personality disorder, or “split personality,” and depersonalization disorder; factitious disorders; sexual and gender disorders, such as sexual dysfunction, gender identity disorder and the paraphilia's; somatic symptom disorders, formerly known as a psychosomatic disorder or somatoform disorder; and combinations thereof.

In some embodiments, the disease, disorder or condition that is treated by activation of a serotonin receptor comprises cognitive impairment; ischemia including stroke; neurodegeneration; refractory substance use disorders; sleep disorders; pain, such as social pain, acute pain, cancer pain, chronic pain, breakthrough pain, bone pain, soft tissue pain, nerve pain, referred pain, phantom pain, neuropathic pain, cluster headaches and migraine; obesity and eating disorders; epilepsies and seizure disorders; neuronal cell death; excitotoxic cell death; or a combination thereof.

In some embodiments, the mental illness is selected from hallucinations and delusions and a combination thereof.

In some embodiments, the hallucinations are selected from visual hallucinations, auditory hallucinations, olfactory hallucinations, gustatory hallucinations, tactile hallucinations, proprioceptive hallucinations, equilibrioceptive hallucinations, nociceptive hallucinations, thermoceptive hallucinations and chronoceptive hallucinations, and a combination thereof.

In some embodiments, the disease, disorder or condition that is treated by activation of a serotonin receptor is psychosis or psychotic symptoms. Accordingly, the present application also includes a method of treating psychosis or psychotic symptoms comprising administering a therapeutically effective amount of one or more compounds of the application to a subject in need thereof.

The present application also includes a use of one or more compounds of the application for treatment of psychosis or psychotic symptoms, as well as a use of one or more compounds of the application for the preparation of a medicament for treatment of psychosis or psychotic symptoms. The application further includes one or more compounds of the application for use in treating psychosis or psychotic symptoms.

In some embodiments, administering to said subject in need thereof a therapeutically effective amount of the compounds of the application does not result in a worsening of psychosis or psychotic symptoms such as, but not limited to, hallucinations and delusions. In some embodiments, administering to said subject in need thereof a therapeutically effective amount of the compounds of the application results in an improvement of psychosis or psychotic symptoms such as, but not limited to, hallucinations and delusions. In some embodiments, administering to said subject in need thereof a therapeutically effective amount of the compounds of the application results in an improvement of psychosis or psychotic symptoms.

In some embodiments, the disease, disorder or condition that is treated by activation of a serotonin receptor is a central nervous system (CNS) disease, disorder or condition and/or a neurological disease, disorder or condition. Accordingly, the present application also includes a method of treating a CNS disease, disorder or condition and/or a neurological disease, disorder or condition that is treated by activation of a serotonin receptor comprising administering a therapeutically effective amount of one or more compounds of the application to a subject in need thereof, that is a subject having the central nervous system (CNS) disease, disorder or condition and/or a neurological disease, disorder or condition. The present application also includes a use of one or more compounds of the application for treatment a CNS disease, disorder or condition and/or a neurological disease, disorder or condition that is treated by activation of a serotonin receptor, as well as a use of one or more compounds of the application for the preparation of a medicament for treatment of a CNS disease, disorder or condition and/or a neurological disease, disorder or condition that is treated by activation of a serotonin receptor. The application further includes one or more compounds of the application for use in treating a CNS disease, disorder or condition and/or a neurological disease, disorder or condition that is treated by activation of a serotonin receptor.

In some embodiments the CNS disease, disorder or condition and/or neurological disease, disorder or condition is selected from neurological diseases including neurodevelopmental diseases and neurodegenerative diseases such as Alzheimer's disease; presenile dementia; senile dementia; vascular dementia; Lewy body dementia; cognitive impairment, Parkinson's disease and Parkinsonian related disorders such as Parkinson's dementia, corticobasal degeneration, and supranuclear palsy; epilepsy; CNS trauma; CNS infections; CNS inflammation; stroke; multiple sclerosis; Huntington's disease; mitochondrial disorders; Fragile X syndrome; Angelman syndrome; hereditary ataxias; neuro-otological and eye movement disorders; neurodegenerative diseases of the retina amyotrophic lateral sclerosis; tardive dyskinesias; hyperkinetic disorders; attention deficit hyperactivity disorder and attention deficit disorders; restless leg syndrome; Tourette's syndrome; schizophrenia; autism spectrum disorders; tuberous sclerosis; Rett syndrome; cerebral palsy; disorders of the reward system including eating disorders such as anorexia nervosa (“AN”) and bulimia nervosa (“BN”); and binge eating disorder (“BED”), trichotillomania, dermotillomania, nail biting; migraine; fibromyalgia; and peripheral neuropathy of any etiology, and combinations thereof.

In some embodiments, the subject is a mammal. In another embodiment, the subject is human. In some embodiments, the subject is a non-human animal. In some embodiments, the subject is canine. In some embodiments, the subject is feline. Accordingly, the compounds, methods and uses of the present application are directed to both human and veterinary diseases, disorders and conditions.

In some embodiments, the “subject in need thereof” is a subject having the disease, disorder or condition to be treated.

In some embodiments, the compounds of the application are useful for treating behavioral problems in subjects that are felines or canines.

Therefore, in some embodiments, the disease, disorder or condition that is treated by activation of a serotonin receptor is behavioral problems in subjects that are felines or canines. Accordingly, the present application also includes a method of treating a behavioral problem comprising administering a therapeutically effective amount of one or more compounds of the application to a non-human subject in need thereof, that is a non-human subject having the behavioral problem. The present application also includes a use of one or more compounds of the application for treatment a behavioral problem in a non-human subject, as well as a use of one or more compounds of the application for the preparation of a medicament for treatment of a behavioral problem in a non-human subject. The application further includes one or more compounds of the application for use in treating a behavioral problem in a non-human subject.

In some embodiments, the behavioral problems are selected from, but are not limited to, anxiety, fear, stress, sleep disturbances, cognitive dysfunction, aggression, excessive noise making, scratching, biting and a combination thereof.

In some embodiments, the non-human subject is canine. In some embodiments, the non-human subject is feline.

The present application also includes a method of treating a disease, disorder or condition by activation of a serotonin receptor comprising administering a therapeutically effective amount of one or more compounds of the application in combination with another known agent useful for treatment of a disease, disorder or condition by activation of a serotonin receptor to a subject in need thereof. The present application also includes a use of one or more compounds of the application in combination with another known agent useful for treatment of a disease, disorder or condition that is treated by activation of a serotonin receptor for treatment of a disease, disorder or condition by activation of a serotonin receptor, as well as a use of one or more compounds of the application in combination with another known agent useful for treatment of a disease, disorder or condition by activation of a serotonin receptor for the preparation of a medicament for treatment of a disease, disorder or condition that is treated by activation of a serotonin receptor. The application further includes one or more compounds of the application in combination with another known agent useful for treatment of a disease, disorder or condition that is treated by activation of a serotonin receptor for use in treating a disease, disorder or condition by activation of a serotonin receptor.

In some embodiments, the disease, disorder or condition that is treated by activation of a serotonin receptor is a mental illness. In some embodiments, the mental illness is selected from hallucinations and delusions and a combination thereof. In some embodiments, the disease, disorder or condition that is treated by activation of a serotonin receptor is a central nervous system (CNS) disorder. In some embodiments, the disease, disorder or condition that is treated by activation of a serotonin receptor is psychosis or psychotic symptoms. In some embodiments, the disease, disorder or condition that is treated by activation of a serotonin receptor is behavioral problems in a non-human subject.

In some embodiments, the disease, disorder or condition that is treated by activation of a serotonin receptor is a mental illness and the one or more compounds of the application are administered in combination with one or more additional treatments for a mental illness. In some embodiments, the additional treatments for a mental illness is selected from antipsychotics, including typical antipsychotics and atypical antipsychotics; antidepressants including selective serotonin reuptake inhibitors (SSRIs) and selective norepinephrine reuptake inhibitors (SNRIs), tricyclic antidepressants, monoamine oxidase inhibitors (MAOIs) (e.g. bupropion), allosteric modulators of the GABA_{A} receptor including, but not limited to inhibitory pregnane neurosteroids, such as zuranolone and brexanolone; anti-anxiety medication including benzodiazepines such as alprazolam; mood stabilizers such as lithium and anticonvulsants such carbamazepine, divalproex (valproic acid), lamotrigine, gabapentin and topiramate. In some embodiments, the disease, disorder or condition that is treated by activation of a serotonin receptor is a mental illness and the one or more compounds of the application are administered in combination with one or more additional treatments for a mental illness. In some embodiments, the additional treatments for a mental illness is selected from antipsychotics, including typical antipsychotics and atypical antipsychotics; antidepressants including selective serotonin reuptake inhibitors (SSRIs) and selective norepinephrine reuptake inhibitors (SNRIs), tricyclic antidepressants and monoamine oxidase inhibitors (MAOIs) (e.g. bupropion); anti-anxiety medication including benzodiazepines such as alprazolam; mood stabilizers such as lithium and anticonvulsants such carbamazepine, divalproex (valproic acid), lamotrigine, gabapentin and topiramate.

In some embodiments, the disease, disorder or condition that is treated by activation of a serotonin receptor is selected from attention deficit hyperactivity disorder and attention deficit disorder and a combination thereof. In some embodiments, the disease, disorder or condition that is treated by activation of a serotonin receptor is attention deficit hyperactivity disorder and/or attention deficit disorder and a combination thereof and the one or more compounds of the application are administered in combination with one or more additional treatments for attention deficit hyperactivity disorder and/or attention deficit disorder and a combination thereof. In some embodiments, the additional treatments for attention deficit hyperactivity disorder and/or attention deficit disorder and a combination thereof are selected from methylphenidate, atomoxetine and amphetamine and a combination thereof.

In some embodiments, the disease, disorder or condition that is treated by activation of a serotonin receptor is dementia or Alzheimer's disease and the one or more compounds of the application are administered in combination with one or more additional treatments for dementia or Alzheimer's disease. In some embodiments, the additional treatments for dementia and Alzheimer's disease are selected acetylcholinesterase inhibitors, NMDA antagonists and muscarinic agonists and antagonists, and nicotinic agonists.

In some embodiments, the acetylcholinesterase inhibitors are selected from donepezil, galantamine, rivastigmine, and phenserine, and combinations thereof.

In some embodiments, the NMDA antagonists are selected from MK-801, ketamine, phencyclidine, and memantine, and combinations thereof.

In some embodiments, the nicotinic agonists is nicotine, nicotinic acid, nicotinic alpha7 agonists or alpha2 beta4 agonists or combinations thereof.

In some embodiments, the muscarinic agonists is a muscarinic M1 agonist or a muscarinic M4 agonist, or combinations thereof.

In some embodiments, the muscarinic antagonist is a muscarinic M2 antagonist.

In some embodiments, the disease, disorder or condition that is treated by activation of a serotonin receptor is psychosis or psychotic symptoms and the one or more compounds of the application are administered in combination with one or more additional treatments for psychosis or psychotic symptoms. In some embodiments, the additional treatments for psychosis or psychotic symptom are selected typical antipsychotics and atypical antipsychotics.

In some embodiments, the typical antipsychotics are selected from acepromazine, acetophenazine, benperidol, bromperidol, butaperazine, carfenazine, chlorproethazine, chlorpromazine, chlorprothixene, clopenthixol, cyamemazine, dixyrazine, droperidol, fluanisone, flupentixol, fluphenazine, fluspirilene, haloperidol, levomepromazine, lenperone, Ioxapine, mesoridazine, metitepine, molindone, moperone, oxypertine, oxyprotepine, penfluridol, perazine, periciazine, perphenazine, pimozide, pipamperone, piperacetazine, pipotiazine, prochlorperazine, promazine, prothipendyl, spiperone, sulforidazine, thiopropazate, thioproperazine, thioridazine, thiothixene, timiperone, trifluoperazine, trifluperidol, triflupromazine and zuclopenthixol and combinations thereof.

In some embodiments, the atypical antipsychotics are selected from amoxapine, amisulpride, aripiprazole, asenapine, blonanserin, brexpiprazole, cariprazine, carpipramine, clocapramine, clorotepine, clotiapine, clozapine, iloperidone, levosulpiride, lurasidone, melperone, mosapramine, nemonapride, olanzapine, paliperidone, perospirone, quetiapine, remoxipride, reserpine, risperidone, sertindole, sulpiride, sultopride, tiapride, veralipride, ziprasidone and zotepine, and combinations thereof.

In some embodiments, the disease, disorder or condition that is treated by activation of a serotonin receptor is a mental illness and the one or more compounds of the application are administered in combination with one or more additional treatments for a mental illness. In some embodiments, the additional treatments for a mental illness is selected typical antipsychotics and atypical antipsychotics.

In some embodiments, effective amounts vary according to factors such as the disease state, age, sex and/or weight of the subject or species. In some embodiments, the amount of a given compound or compounds that will correspond to an effective amount will vary depending upon factors, such as the given drug(s) or compound(s), the pharmaceutical formulation, the route of administration, the type of condition, disease or disorder, the identity of the subject being treated and the like, but can nevertheless be routinely determined by one skilled in the art.

In some embodiment, the compounds of the application are administered one, two, three or four times a year. In some embodiments, the compounds of the application are administered at least once a week. However, in another embodiment, the compounds are administered to the subject from about one time per two weeks, three weeks or one month. In another embodiment, the compounds are administered about one time per week to about once daily. In another embodiment, the compounds are administered 1, 2, 3, 4, 5 or 6 times daily. The length of the treatment period depends on a variety of factors, such as the severity of the disease, disorder or condition, the age of the subject, the concentration and/or the activity of the compounds of the application and/or a combination thereof. It will also be appreciated that the effective dosage of the compound used for the treatment may increase or decrease over the course of a particular treatment regime. Changes in dosage may result and become apparent by standard diagnostic assays known in the art. In some instances, chronic administration is required. For example, the compounds are administered to the subject in an amount and for duration sufficient to treat the subject.

In some embodiments, the compounds of the application are administered at doses that are hallucinogenic or psychotomimetic and taken in conjunction with psychotherapy or therapy and may occur once, twice, three, or four times a year. However, in some embodiments, the compounds are administered to the subject once daily, once every two days, once every 3 days, once a week, once every two weeks, once a month, once every two months, or once every three months at doses that are not hallucinogenic or psychotomimetic.

A compound of the application is either used alone or in combination with other known agents useful for treating diseases, disorders or conditions by activation of a serotonin receptor, such as the compounds of the application. When used in combination with other known agents useful in treating diseases, disorders by activation of a serotonin receptor, it is an embodiment that a compound of the application is administered contemporaneously with those agents. As used herein, “contemporaneous administration” of two substances to a subject means providing each of the two substances so that they are both active in the individual at the same time. The exact details of the administration will depend on the pharmacokinetics of the two substances in the presence of each other and can include administering the two substances within a few hours of each other, or even administering one substance within 24 hours of administration of the other, if the pharmacokinetics are suitable. Design of suitable dosing regimens is routine for one skilled in the art. In particular embodiments, two substances will be administered substantially simultaneously, i.e., within minutes of each other, or in a single composition that contains both substances. It is a further embodiment of the present application that a combination of agents is administered to a subject in a non-contemporaneous fashion. In some embodiments, a compound of the present application is administered with another therapeutic agent simultaneously or sequentially in separate unit dosage forms or together in a single unit dosage form. Accordingly, the present application provides a single unit dosage form comprising one or more compounds of the application, an additional therapeutic agent and a pharmaceutically acceptable carrier.

The dosage of a compound of the application varies depending on many factors such as the pharmacodynamic properties of the compound, the mode of administration, the age, health and weight of the recipient, the nature and extent of the symptoms, the frequency of the treatment and the type of concurrent treatment, if any and the clearance rate of the compound in the subject to be treated. One of skill in the art can determine the appropriate dosage based on the above factors. In some embodiments, one or more compounds of the application are administered initially in a suitable dosage that is adjusted as required, depending on the clinical response. Dosages will generally be selected to maintain a serum level of the one or more compounds of the application from about 0.01 μg/cc to about 1000 μg/cc, or about 0.1 μg/cc to about 100 μg/cc. As a representative example, oral dosages of one or more compounds of the application will range between about 10 μg per day to about 1000 mg per day for an adult, suitably about 10 μg per day to about 500 mg per day, more suitably about 10 μg per day to about 200 mg per day. For parenteral administration, a representative amount is from about 0.0001 mg/kg to about 10 mg/kg, about 0.0001 mg/kg to about 1 mg/kg, about 0.01 mg/kg to about 0.1 mg/kg or about 0.0001 mg/kg to about 0.01 mg/kg will be administered. For oral administration, a representative amount is from about 0.001 μg/kg to about 10 mg/kg, about 0.1 μg/kg to about 10 mg/kg, about 0.01 μg/kg to about 1 mg/kg or about 0.1 μg/kg to about 1 mg/kg. For administration in suppository form, a representative amount is from about 0.1 mg/kg to about 10 mg/kg or about 0.1 mg/kg to about 1 mg/kg. In some embodiments of the application, compositions are formulated for oral administration and the one or more compounds are suitably in the form of tablets containing 0.1, 0.25, 0.5, 0.75, 1.0, 5.0, 10.0, 20.0, 25.0, 30.0, 40.0, 50.0, 60.0, 70.0, 75.0, 80.0, 90.0, 100.0, 150, 200, 250, 300, 350, 400, 450, 500, 550, 600, 650, 700, 750, 800, 850, 900, 950 or 1000 mg of active ingredient (one or more compounds of the application) per tablet. In some embodiments of the application the one or more compounds of the application are administered in a single daily, weekly or monthly dose or the total daily dose is divided into two, three or four daily doses.

In some embodiments, the compounds of the application are used or administered in an effective amount which comprises administration of doses or dosage regimens that are devoid of clinically meaningful psychedelic/psychotomimetic actions. In some embodiments, the compounds of the application are used or administered in an effective amount which comprises administration of doses or dosage regimens that provide clinical effects similar to those exhibited by a human plasma psilocin Cmax of 4 ng/mL or less and/or human 5-HT_{2A} human CNS receptor occupancy of 40% or less or those exhibited by a human plasma psilocin Cmax of 1 ng/mL or less and/or human 5-HT_{2A} human CNS receptor occupancy of 30% or less. In some embodiments, the compounds of the application are used or administered in an effective amount which comprises administration of doses or dosage regimens that provide clinical effects similar to those exhibited by a human plasma psilocin Tmax in excess of 60 minutes, in excess of 120 minutes or in excess of 180 minutes.

To be clear, in the above, the term “a compound” also includes embodiments wherein one or more compounds are referenced. Likewise, the term “compounds of the application” also includes embodiments wherein only one compound is referenced.

### V. Preparation of Compounds

Compounds of the present application can be prepared by various synthetic processes. The choice of particular structural features and/or substituents may influence the selection of one process over another. The selection of a particular process to prepare a given compound of the application is within the purview of the person of skill in the art. Some starting materials for preparing compounds of the present application are available from commercial chemical sources or may be extracted from cells, plants, animals or fungi. Other starting materials, for example as described below, are readily prepared from available precursors using straightforward transformations that are well known in the art. In the Schemes below showing some embodiments of methods of preparation of compounds of the application, all variables are as defined in Formula I, unless otherwise stated.

In some embodiments of the application, the compounds of the application are generally prepared according to the process illustrated in Schemes I-Ill.

In some embodiments, the compounds of Formula (I) wherein Q is P(O)OR⁹ and R⁹ is H are prepared as shown in Scheme 1. Therefore, a compound of Formula A is reacted with a phosphorylating agent such as phosphoryl chloride in the presence of a suitable base such as triethylamine in a suitable solvent such as dichloromethane to provide the compounds of Formula I.

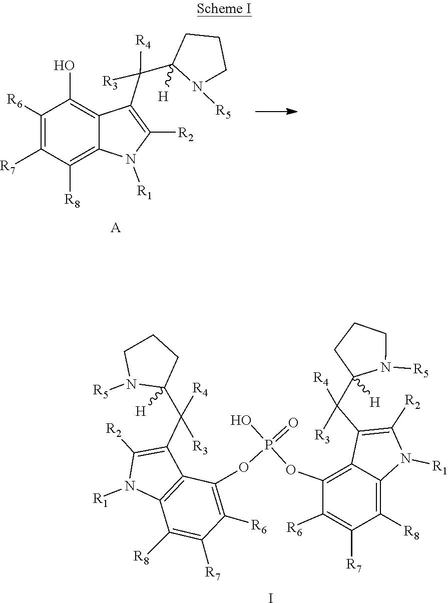

In some embodiments, as shown in Scheme I, compounds of Formula I are provided using, for example, reaction conditions found in CN 102382135 (Faming Zhuanli Shenqing) and Mondal et al, Tetrahedron Letters, 58(25), 2460-2464; 2017.

In some embodiments, the compounds of Formula (I) wherein Q is C₁-C₄alkylene-P(O)OR⁹—C₁-C₆alkylene and R⁹ is H are prepared as shown in Scheme II. Therefore, a compound of Formula A is reacted with a compound of Formula B in the presence of a suitable base, such as sodium base, in a suitable solvent such as dimethyl formamide at a suitable temperature such as about 140° C. to provide the compounds of Formula I.

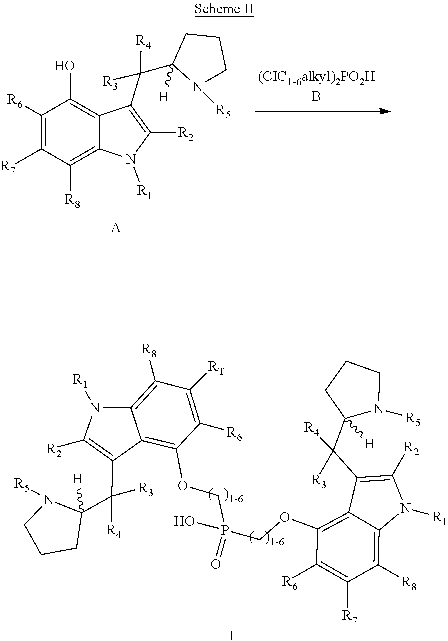

In some embodiments, as shown in Scheme II, the compounds of Formula (I) are provided using, for example, reaction conditions found in Mukhametzyanova et al., Izvestiya Akademii Nauk SSSR, Seriya Khimicheskaya, Issue: 2, Pages: 373-80, 1969.

In some embodiments, the compounds of Formula (I) wherein Q is C(O)Q′C(O are prepared as shown in Scheme Ill. Therefore, a compound of Formula A is reacted with a compound of Formula C in the presence of a suitable base such as triethylamine in a suitable solvent such as dichloromethane at a suitable temperature such as about 0° C. to about room temperature (e.g. about 18° C. to about 25° C.) to provide the compounds of Formula I.

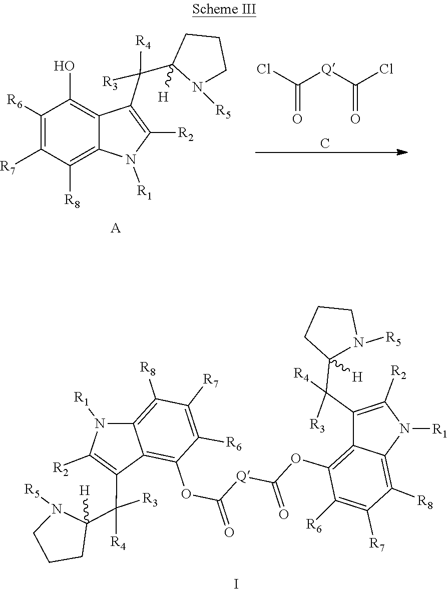

In some embodiments, as shown in Scheme Ill, the compounds of Formula (I) are provided using, for example, reactions conditions found Derosa J. et al., Journal of the American Chemical Society, 2021 Volume 143, Issue 25, pp 9303-9307.

In some embodiments, as shown in Scheme Ill, the compounds of Formula (I) are provided using, for example the reaction conditions found in Gerasimov et al., J. Med. Chem. 1999, 42, 4257-4263 and/or Macor et al. J. Med. Chem. 1992, 35, 4503-4505.

In some embodiments, the compounds of Formula I wherein Q is C(O)OQ′OC(O) or C(O)NR^{9′}Q′NR^{9′}C(O) are prepared as shown in Scheme III except with using the appropriate compound of Formula C.

In some embodiments, the compounds of Formula I wherein Q is C(O) are prepared as shown in Scheme IV. Therefore, a compound of Formula A is reacted with a compound of Formula D (triphosphene) in the presence of a suitable base such as triethylamine and sodium hydroxide in a suitable solvent such as dichloromethane and H₂O at a suitable temperature such as with heating (e.g. greater than about 25° C.) to provide the compounds of Formula I.

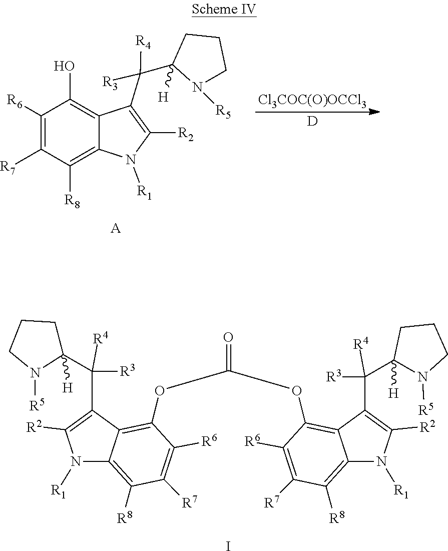

In some embodiments, as shown in Scheme IV, the compounds of Formula I are provided using, for example, reactions conditions found in Guangzhou Huagong, 39(14), 81-82; 201.

In some embodiments, the compounds of Formula (I) wherein Q is SO₂ are prepared as shown in Scheme V. Therefore, a compound of Formula A is reacted with a N,N′-sulfuryldiimidazole D1 in the presence of a suitable base such as cesium carbonate in a suitable solvent such as tetrahydrofuran at a suitable temperature such as about reflux to about room temperature (e.g. about 70° C. to about 25° C.) to provide the compounds of Formula I and/or using sulfuryl chloride D2.

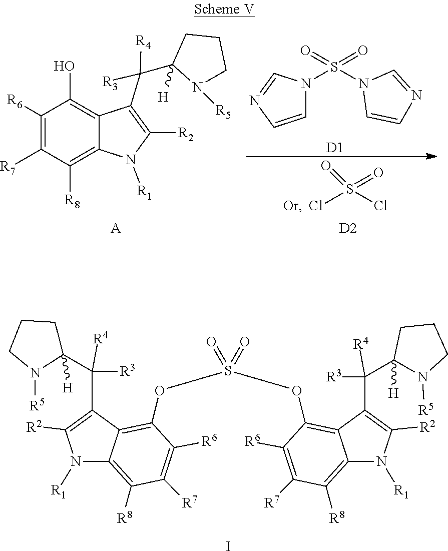

In some embodiments, as shown in Scheme V, the compounds of Formula (I) are provided using, for example the reaction conditions found in Guan, Bing-Tao et al., Organic Letters, 12(2), 396-399; 2010 and/or Younker, Jarod M., Journal of Organic Chemistry, 69(26), 9043-9048; 2004.

In some embodiments, the compounds of Formula A are prepared using known methods, for example using the synthetic procedures found in WO2021/155467A1 (Mindset Pharma Inc.).

A person skilled in the art would appreciate that further manipulation of the substituent groups using known chemistry can be performed on the intermediates and final compounds in the Schemes above to provide alternative compounds of the application.

Salts of compounds of the application may be formed by methods known to those of ordinary skill in the art, for example, by reacting a compound of the application with an amount of acid or base, such as an equivalent amount, in a medium such as one in which the salt precipitates or in aqueous medium followed by lyophilization.

The formation of solvates will vary depending on the compound and the solvate. In general, solvates are formed by dissolving the compound in the appropriate solvent and isolating the solvate by cooling or using an antisolvent. The solvate is typically dried or azeotroped under ambient conditions. The selection of suitable conditions to form a particular solvate can be made by a person skilled in the art. Examples of suitable solvents are ethanol, water and the like. When water is the solvent, the molecule is referred to as a “hydrate”. The formation of solvates of the compounds of the application will vary depending on the compound and the solvate. In general, solvates are formed by dissolving the compound in the appropriate solvent and isolating the solvate by cooling or using an antisolvent. The solvate is typically dried or azeotroped under ambient conditions. The selection of suitable conditions to form a particular solvate can be made by a person skilled in the art.

Isotopically-enriched compounds of the application and pharmaceutically acceptable salts, solvates and/or prodrug thereof, can be prepared without undue experimentation by conventional techniques well known to those skilled in the art or by processes analogous to those described in the Schemes and Examples herein using suitable isotopically-enriched reagents and/or intermediates.

Throughout the processes described herein it is to be understood that, where appropriate, suitable protecting groups will be added to and subsequently removed from, the various reactants and intermediates in a manner that will be readily understood by one skilled in the art. Conventional procedures for using such protecting groups as well as examples of suitable protecting groups are described, for example, in “Protective Groups in Organic Synthesis”, T. W. Green, P. G. M. Wuts, Wiley-Interscience, New York, (1999). It is also to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities and ways to circumvent them by carrying out appropriate transformations and synthetic steps in a suitable order, will be readily understood to one skilled in the art. Examples of transformations are given herein and it is to be understood that the described transformations are not limited only to the generic groups or substituents for which the transformations are exemplified. References and descriptions of other suitable transformations are given in “Comprehensive Organic Transformations—A Guide to Functional Group Preparations” R. C. Larock, VHC Publishers, Inc. (1989). References and descriptions of other suitable reactions are described in textbooks of organic chemistry, for example, “Advanced Organic Chemistry”, March, 4^{th} ed. McGraw Hill (1992) or, “Organic Synthesis”, Smith, McGraw Hill, (1994).

Nucleophilic displacement reaction conditions comprise any known method for the reaction of a nucleophile to displace a leaving group to form a bond that is compatible with the intermediates and products shown in the above Schemes or that may be used to prepare a compound of the application. In some embodiments, such conditions comprise combining reactants in the presence of a base in a suitable solvent.

Techniques for purification of intermediates and final products include, for example, straight and reversed phase chromatography on column or rotating plate, recrystallisation, distillation and liquid-liquid or solid-liquid extraction, which will be readily understood by one skilled in the art.

### EXAMPLES

The following non-limiting examples are illustrative of the present application.

### A. Synthetic Protocols

### General Methods

All starting materials used herein were commercially available or earlier described in the literature. The ¹H and ¹³C NMR spectra were recorded either on Bruker 300, Bruker DPX400 or Varian +400 spectrometers operating at 300, 400 and 400 MHz for ¹H NMR respectively, using TMS or the residual solvent signal as an internal reference, in deuterated chloroform as solvent unless otherwise indicated. All reported chemical shifts are in ppm on the delta-scale, and the fine splitting of the signals as appearing in the recordings is generally indicated, for example as s: singlet, br s: broad singlet, d: doublet, t: triplet, q: quartet, m: multiplet. Unless otherwise indicated, in the tables below, ¹H NMR data was obtained at 400 MHz, using CDCl₃ as the solvent.

Purification of products was carried out using Chem Elut Extraction Columns (Varian, cat #1219-8002), Mega BE-SI (Bond Elut Silica) SPE Columns (Varian, cat #12256018; 12256026; 12256034) or by flash chromatography in silica-filled glass columns.

Synthesis of representative Exemplary Compounds of the Application

The following compounds were prepared using one or more of the synthetic methods outlined in Schemes I to III:

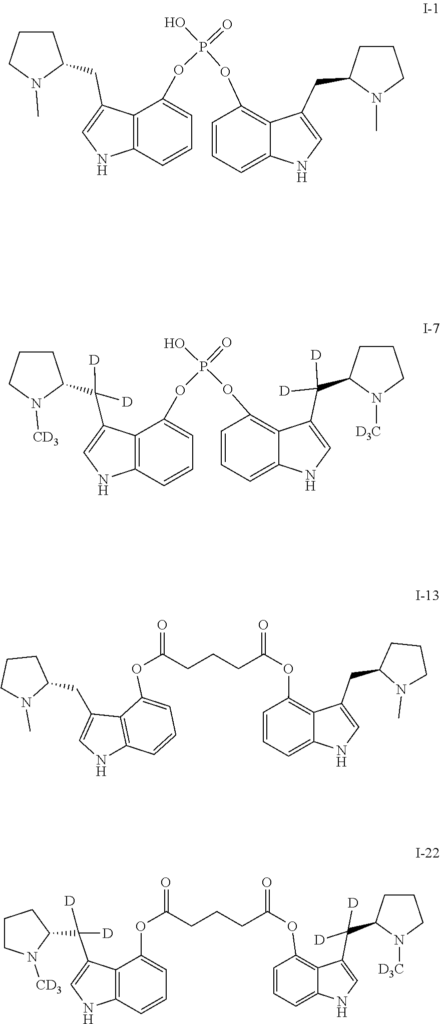 [SMILES: CN1CCC[C@@H]1Cc1c[nH]c2cccc(OP(=O)(O)Oc3cccc4[nH]cc(C[C@H]5CCCN5C)c34)c12 ; [2H]C([2H])(c1c[nH]c2cccc(OP(=O)(O)Oc3cccc4[nH]cc(C([2H])([2H])[C@H]5CCCN5C([2H])([2H])[2H])c34)c12)[C@H]1CCCN1C([2H])([2H])[2H] ; CN1CCC[C@@H]1Cc1c[nH]c2cccc(OC(=O)CCCC(=O)Oc3cccc4[nH]cc(C[C@H]5CCCN5C)c34)c12 ; [2H]C([2H])(c1c[nH]c2cccc(OC(=O)CCCC(=O)Oc3cccc4[nH]cc(C([2H])([2H])[C@H]5CCCN5C([2H])([2H])[2H])c34)c12)[C@H]1CCCN1C([2H])([2H])[2H]]

### Example 1: Bis(3-(((R)-1-methylpyrrolidin-2-yl)methyl)-1H-indol-4-yl) glutarate (I-13)

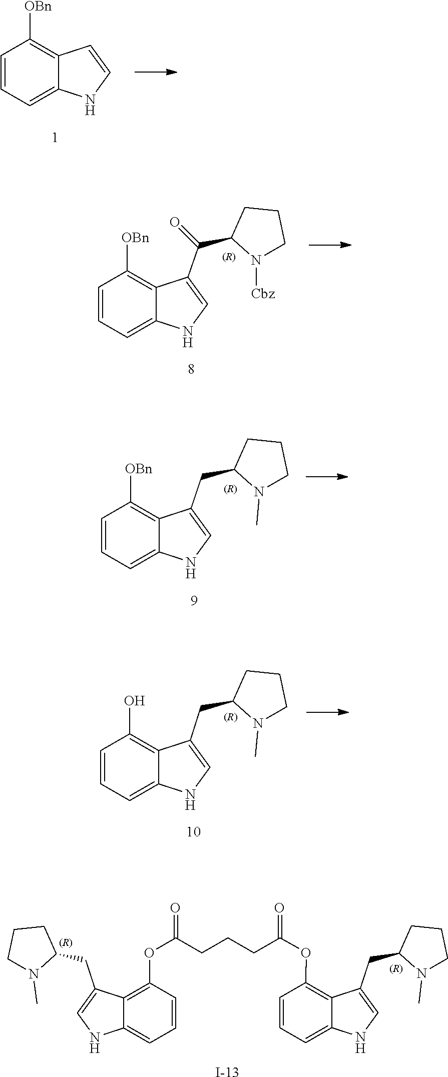

### Synthesis of (R)-2-(2-(4-(benzyloxy)-1H-indole-3-carbonyl)pyrrolidin-1-yl)-1-phenyl-212-ethan-1-one (8)

A solution of (2-oxo-2-phenyl-1I²-ethyl)-D-proline (1.67 g, 6.71 mmol) in dry THF (20 mL) was treated with thionyl chloride (0.98 mL, 13.434 mmol) at 0° C. The reaction was brought to room temperature, then refluxed for 2 h. The reaction was brought to room temperature, solvent was evaporated and crude was dried under vacuum to obtain the corresponding acid chloride.

A solution of 4-(benzyloxy)-1H-indole (1.5 g, 6.71 mmol) in dry CH₂Cl₂ (50 mL) at 5-10° C. was treated with above crude acid chloride in dry CH₂Cl₂ (10 mL) and ethyl magnesium bromide (4.48 mL, 13.434 mmol, 3 M in THF) simultaneously over a period of 15 min. and stirred at same temperature for further 15 min. The reaction was quenched with con. HCl (10 mL) followed by water (50 mL) and product was extracted into CH₂Cl₂ (2×50 mL). CH₂Cl₂ layer was washed with sat. NaHCO₃ solution (50 mL), brine (25 mL) and dried (Na₂SO₄). Solvent was evaporated and crude was purified by flash column chromatography (CH₂Cl₂ to EtOAc:CH₂Cl₂, 1:4) on silica gel to obtain to obtain the title compound 8 (1.41 g, 46%) as a white foam. ¹H NMR (CDCl₃): δ 9.90, 9.30 (2s, 1H), 7.72, 7.70 (2d, 1H, J=3.0 Hz), 7.54-7.02 (m, 12H), 6.59, 6.57 (2d, 1H, J=3.0 Hz), 5.59-5.55 (m, 1H), 5.32-4.96 (m, 4H), 3.67-3.60 (m, 1H), 3.50-3.30 (m, 1H), 1.90-1.50 (m, 4H); ESI-MS (m/z, %): 477 (M+Na, 100), 455 (MH⁺).

### Synthesis of (R)-4-(benzyloxy)-3-((1-methylpyrrolidin-2-yl)methyl)-1H-indole (9)

A suspension of lithium aluminum hydride (0.63 g, 16.720 mmol) in dry THF (10 mL) was treated with (R)-2-(2-(4-(benzyloxy)-1H-indole-3-carbonyl)pyrrolidin-1-yl)-1-phenyl-2I²-ethan-1-one (1.52 g, 3.344 mmol) in dry THF (25 mL) at 0° C. over a period of 10 min. The reaction was brought to room temperature, then refluxed for additional 16 hours. The reaction was worked-up and purified as described for compound 3 to obtain the title compound 9 (0.858 g, 80%) as an off-white solid. ¹H NMR (CDCl₃): δ 8.07 (s, 1H), 7.52-7.29 (m, 5H), 7.07-6.91 (m, 3H), 6.58-6.55 (m, 1H), 5.25-5.17 (m, 2H), 3.51-3.44 (m, 1H), 3.08-3.03 (m, 1H), 2.62-2.50 (m, 2H), 2.15-2.10 (m, 1H), 2.08 (s, 3H), 1.68-1.57 (m, 4H).

### Synthesis of (R)-3-((1-methylpyrrolidin-2-yl)methyl)-1H-indol-4-ol (10)

A solution of (R)-4-(benzyloxy)-3-((1-methylpyrrolidin-2-yl)methyl)-1H-indole (0.87 g, 2.71 mmol) in methanol (25 mL) was treated with 10% palladium on carbon (0.15 g, 10%) and hydrogenated under balloon pressure for 2 h. The reaction was filtered through a pad celite and washed with methanol (2×20 mL). Combined methanol layer was evaporated and crude was purified by flash column chromatography (2 M NH₃ in MeOH:CH₂Cl₂, 5:95) on silica gel to obtain the title compound 10 (0.52 g, 85%) as an off-white solid. ¹H NMR of TFA salt (DMSO-d₆): δ 10.81 (s, 1H), 9.61 (brs, 1H), 7.06 (d, 1H, J=3.0 Hz), 6.87-6.79 (m, 2H), 6.36 (dd, 1H, J=3.0 Hz), 3.70-3.58 (m, 2H), 3.46-3.41 (m, 1H), 3.16-3.09 (m, 1H), 2.98-2.84 (m, 4H), 2.06-1.76 (m, 4H); ESI-MS (m/z, %): 231 (MH⁺, 100).

### Synthesis of bis(3-(((R)-1-methylpyrrolidin-2-yl)methyl)-1H-indol-4-yl) glutarate (I-13)

A solution of (R)-3-((1-methylpyrrolidin-2-yl)methyl)-1H-indol-4-ol (1.28 g, 5.59 mmol) in dry THF (50 mL) was treated with Et₃N (1.41 mL, 10.16 mmol), followed by glutaroyl dichloride (0.32 mL, 2.54 mmol) in dry THF (20 mL) at 0° C. over a period of 10 min. The reaction was brought to room temperature and stirred overnight (16 h). Reaction was quenched with water (100 mL), product was extracted into ethyl acetate (2×100 mL). Combined organic layer was washed with brine (25 mL) and dried (Na₂SO₄). Solvent was evaporated and crude was purified by flash column chromatography (2 M NH₃ in MeOH:CH₂Cl₂, 5:95 to 1:9) on silica gel to obtain the title compound I-13 (0.42 g, 29.6%) as a pale-yellow oil, which was converted to dihydrochloride salt with 2 M HCl in ether. ¹H NMR (HCl salt, DMSO-d₆): δ 11.31 (s, 2H), 10.85 (s, 2H), 7.33-7.27 (m, 4H), 7.11-7.06 (m, 2H), 6.81-6.74 (m, 2H), 3.63-3.59 (m, 3H), 3.42-3.31 (m, 3H), 3.12-3.02 (m, 6H), 2.81-2.73 (m, 6H), 2.52-2.48 (m, 2H), 2.16-1.71 (m, 10H); ESI-MS (m/z, %): 557 (MH⁺, 100).

### Example 2: Bis Bis(3-(((R)-1-(methyl-d₃)pyrrolidin-2-yl)methyl-d₂)-1H-indol-4-yl) glutarate (I-22)

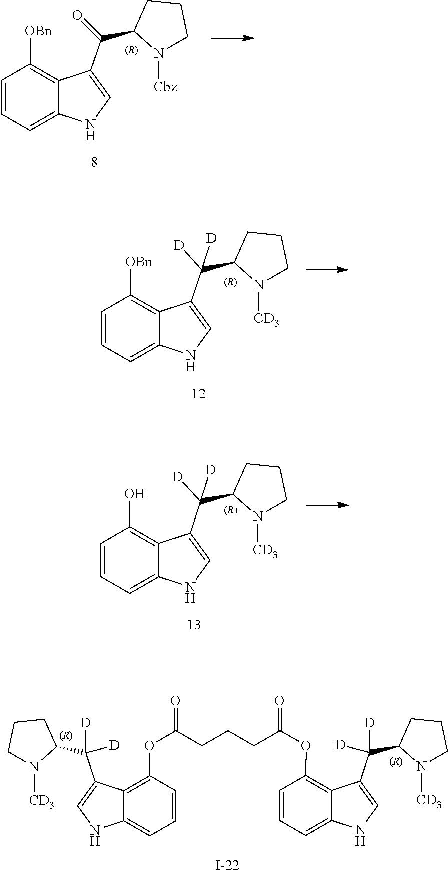

### Synthesis of (R)-4-(benzyloxy)-3-((1-(methyl-d₃)pyrrolidin-2-yl)methyl-d₂)-1H-indole (12)

Prepared from (R)-2-(2-(4-(benzyloxy)-1H-indole-3-carbonyl)pyrrolidin-1-yl)-1-phenyl-2I²-ethan-1-one (1.41 g, 2.58 mmol) as described for compound 3 to obtain the title compound 12 (0.98 g, 97%) as a white foam. ¹H NMR (DMSO-d₆): δ 10.77 (s, 1H), 7.54-7.51 (m, 2H), 7.42-7.32 (m, 3H), 6.98-6.90 (m, 3H), 6.54-6.50 (m, 1H), 5.20-5.13 (m, 2H), 2.91-2.86 (m, 1H), 2.40-2.36 (m, 1H), 2.06-2.00 (m, 1H), 1.61-1.45 (m, 4H); ESI-MS (m/z, %): 326 (MH⁺, 100).

### Synthesis of (R)-3-((1-(methyl-d₃)pyrrolidin-2-yl)methyl-d₂)-1H-indol-4-ol (13)

A solution of (R)-2-(2-(4-(benzyloxy)-1H-indole-3-carbonyl)pyrrolidin-1-yl)-1-phenyl-2I²-ethan-1-one (0.95 g, 2.91 mmol) in dry methanol (20 mL) was treated with Pd—C (0.25 g) and hydrogenated under hydrogen atm. for an additional 2 h. The reaction was filtered through a pad of celite and washed with methanol (2×15 mL). Combined methanol layer was evaporated, and crude was purified by flash column chromatography (2M NH₃ in MeOH:CH₂Cl₂, 5:95) on silica gel to obtain the title compound 13 (0.6 g, 87%) as a white foam. ¹H NMR of TFA salt (DMSO-d₆): δ 10.80 (s, 1H), 9.54 (brs, 1H), 7.11-7.06 (m, 1H), 6.87-6.79 (m, 2H), 6.37 (d, 1H, J=3.0 Hz), 3.68-3.57 (m, 2H), 3.16-3.07 (m, 1H), 2.11-1.73 (m, 4H); ESI-MS (m/z, %): 236 (MH⁺, 100).

### Synthesis of bis(3-(((R)-1-(methyl-d₃)pyrrolidin-2-yl)methyl-d₂)-1H-indol-4-yl) glutarate (I-22)

A solution of (R)-3-((1-(methyl-d₃)pyrrolidin-2-yl)methyl-d₂)-1H-indol-4-ol (1.38 g, 5.86 mmol) in dry THF (50 mL) was treated with Et₃N (1.55 mL, 11.16 mmol), followed by glutaroyl dichloride (0.35 mL, 2.79 mmol) in dry THF (20 mL) at 0° C. over a period of 10 min. The reaction was brought to room temperature and stirred overnight (16 h). Reaction was worked-up and purified as described for compound I-13 to obtain the title compound I-22 (0.8 g, 50.6%) as a pale-yellow oil, which was converted to dihydrochloride salt with 2 M HCl in ether. ¹H NMR (HCl salt, DMSO-d₆): δ 11.31 (s, 2H), 10.80 (s, 2H), 7.33-7.27 (m, 4H), 7.11-7.07 (m, 2H), 6.81-6.74 (m, 2H), 3.64-3.56 (m, 3H), 3.42-3.37 (m, 2H), 3.12-3.02 (m, 3H), 2.52-2.48 (m, 2H), 2.13-1.75 (m, 10H); ESI-MS (m/z, %): 567 (MH+, 100).

The following compounds were prepared using one or more of the synthetic methods outlined in Schemes I to III and Examples 1 and 2:

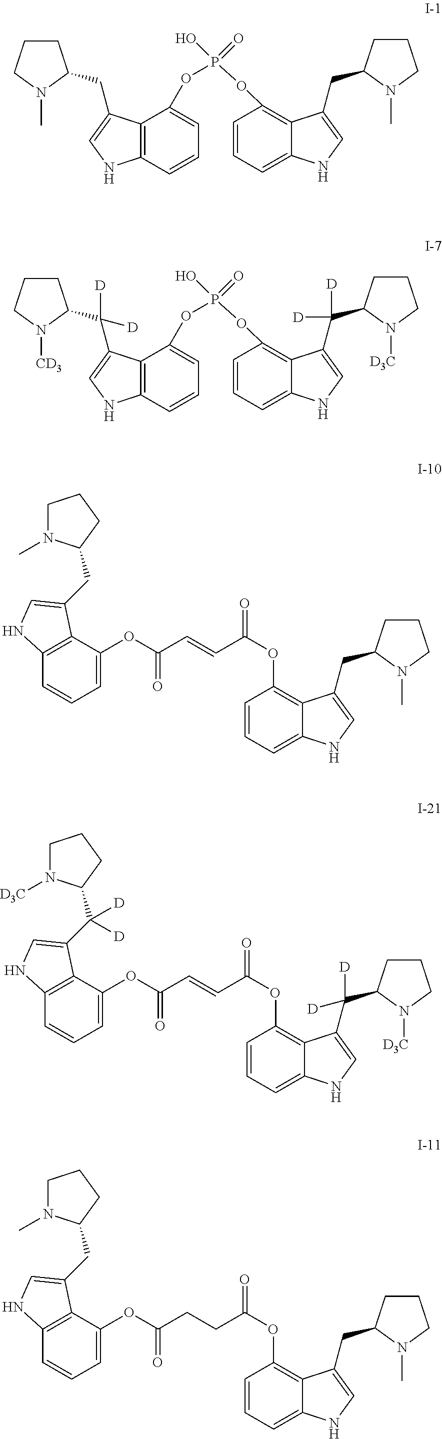

### B. Biological Testing

### Example 3: Human 5-HT2A: Functional FLIPR Assay

### Objective:

The potential excitatory effects of compounds targeting on human 5-hydroxytryptamine receptor 2A (5-HT2A) under agonist mode was assessed.

### 1 Materials and Instrumentation

1.1 Cell line

| Cell line Name | Target | Host cell |
|---|---|---|
| HTR2A&Gα15-HEK293 | 5HT2A | Flp-In-293 |

1.2 Materials

| Regents | Vendor | Cat# |
|---|---|---|
| DMEM | Gibco | 10569-010 |
| Dialyzed FBS | BIOSUN | BS-0005-500 |
| Penicillin-Streptomycin | Invitrogen | 15140 |
| Hygromycin B | Invivogen | Ant-hg-5 |
| Tetracycline | Abcam | ab141223 |
| hydrochloride | | |
| TrypLE ™Express | Gibco | 12604-013 |
| DPBS | Gibco | 14190250 |
| DMSO | Millipore | 1029312500 |
| Probenecid | Sigma | P8761 |
| FLIPR Calcium 6 Assay | Molecular Device | R8191 |
| Kit | | |
| HEPES | Invitrogen | 15630 |
| Hank's Buffered Saline | Invitrogen | 14025 |
| Solution | | |
| Serotonin HCl | Selleck | S4244 |

1.3 Instrumentation and consumables

| Item | Supplier | Cat# |
|---|---|---|
| Fluorometric Imaging Plate Reader | Molecular | Tetra |
| (FLIPR) | Device | |
| Countess Automated Cell Counter | Invitrogen | Countess |
| Cell Counting Chamber Slides | Invitrogen | C10312 |
| STERI-CYCLE CO2 Incubator | Thermo | 371 |
| 1300 Series Class II Biological Safety | Thermo | 1389 |
| Cabinet | | |
| Table-type Large Capacity Low Speed | Cence | L550 |
| Centrifuge | | |
| Centrifuge | Eppendorf | 5702 |
| Echo | Labcyte | 550 |
| Echo | Labcyte | 655 |
| Electro-thermal incubator | Shanghai | DHP-9031 |
| | Yiheng | |
| plate shaker | IKA | MS3 digital |
| Water Purification System | ULUPURE | UPH-III-20T |
| Versatile and Universal pH and | Mettler Toledo | S220 |
| Conductivity Meters | | |
| 384-Well plate | Corning | 3764 |
| 384-Well LDV Clear microplate | LABCYTE | LP-0200 |
| 384-Well Polypropylene microplate | LABCYTE | PP-0200 |
| 384-well compound plate | Corning | 3657 |
| T25 cell culture flask | Corning | 430639 |
| 50 mL Polypropylene Centrifuge Tube | JET | CFT011500 |
| 15 mL Polypropylene Centrifuge Tube | JET | CFT011150 |

### 2 Experimental Methods

### 2.1 Cell Culture

HTR2A&Gα15-HEK293 cells were cultured in DMEM medium containing 10% dialyzed FBS and 1×penicillin-streptomycin, 100 μg/mL Hygromycin B and 300 μg/mL G418. The cells were passaged about three times a week, maintained between ~30% to ~90% confluence.

### 2.2 Cell Plating

1. The cell culture medium (DMEM medium containing 10% dialyzed FBS and 1×penicillin-streptomycin, 100 μg/mL Hygromycin B and 300 μg/mL G418), TrypLE™ Express and DPBS to R.T. was warmed in advance.
2. For induction, 1 μg/ml tetracycline (final concentration) was added to cell culture medium and incubated for 48 hours prior to seeding cells into plate at 37° C., 5% (v/v) CO₂. The cell culture medium was removed from flask. Cells were washed with DPBS.
3. 2 mL TrypLE™ Express was added to the flask, mixed well by gentle shaking and cells were incubated at 37° C. for a few minutes.
4. The cells were checked for morphological change under microscope, the digestion was stopped by adding 4 mL cell culture medium to the flask when most of cells turned to round.
5. The cell suspension was transferred into a 15 mL centrifuge tube, and then centrifuged at 1,200 rpm for 5 minutes.
6. The supernatant was removed. The cell pellet was resuspended with 2 mL cell culture medium.
7. The cell density was counted using cell counter. Only cells with >85% viability were used for the assay.
8. Cells were diluted to 6.67×10⁵/mL with cell culture medium.
9. 30 μL/well cell suspensions added into a 384-well cell plate (The cell density was 20,000 cells/well).
10. The cell plate was incubated overnight at 37° C., 5% (v/v) CO₂.

### 2.3 Cell Handling

On the day of experiments, culture medium was removed from the cell plate.

10 μL of assay buffer (20 mM HEPES, in 1×HBSS, pH 7.4) was added to each well of the cell plate.

2×dye solution was prepared following the manual of the FLIPR® Calcium 6 Assay Kit:
i. The dye was diluted with assay buffer.
ii. Probenecid was added to the final concentration of 5 mM.
iii. Vortexed vigorously for 1-2 minutes.
4. 10 μL of 2×dye solution was added to each well of the cell plate
5. The cell plate was placed on plate shaker, followed by shaking at 600 rpm for 2 minutes.
6. The plate was incubated at 37° C. for 2 hours followed by an additional 15-minute incubation at 25° C.
   2.4 Prepare 3×compound
1. Serotonin HCl was prepared to the concentration of 10 mM with DMSO.
2. The test compounds were prepared to the concentration of 10 mM with DMSO.
3. The compounds were added to a 384-well compound source plate.
4. 3-folds serial dilutions were performed with DMSO.
5. 90 nL/well of serial diluted compounds was transferred from source plate to a 384-well compound plate by using an Echo.
6. 30 μL/well of assay buffer (20 mM HEPES in 1×HBSS, pH 7.4) was added to the compound plate.
7. The plate was mixed on-plate shaker for 2 minutes.

### 2.5 FLIPR Assay

1. After the cells were incubated with dye solution, the cell plate, compound plate containing 3×compounds and FLIPR tips were placed into FLIPR.
2. 10 μL of 3×compounds transferred from the compound plate to the cell plate by FLIPR.
3. The plate was read for 160 sec with 1 sec interval and the data of agonist mode was obtained.

### 3 Data Analysis

1. The normalized fluorescence reading (RFU) was calculated as shown below, wherein Fmax and Fmin stand for maximum and minimum of calcium signal during defined time window:

$$RFU = F max - F min$$

2. EC₅₀ by fitting RFU against log of compound concentrations with Hill equation was calculated using XLfit.

### Results & Discussion

The results of potential competition binding properties of the exemplary dimer compound (I-13 and I-22) of the application and their respective metabolites (10 and 13, see examples 1 and 2 respectively) targeting the human 5-hydroxytryptamine receptor 2A (5-HT2A) are summarized in Table 2. The results of exemplary compounds of the application are presented as IC₅₀ is provided in Table 2.

TABLE 2
Effect of exemplary compounds of Formula I using
FLIPR functional assay on human 5-HT2A receptor

| Compound ID# | h5-HT2A, EC₅₀ [nM] | RFU@10 μM ⁽¹⁾ |
|---|---|---|
| Psilocybin | ND⁽²⁾ | 257 |
| Psilocin | 72.5 | 308 |
| I-13 (Example 1) | 32.33 | 3887 |
| 10 (Metabolite of I-13) | 47 | 2929 |
| I-22 (Example 2) | 13.06 | 5409 |
| 13 (Metabolite of I-22) | 44 | 3108 |

⁽¹⁾ Curve fitting with activation (%) @ 10 mM with RFU
⁽²⁾ND: not detected

Exemplary compounds of Formula I were evaluated using radioligand binding assay on human 5-HT2A receptor. EC₅₀ (nM) concentrations are illustrated in Table 2. This assay confirms that compounds or metabolites of the application are effective ligands of the target human 5-HT2A receptors.

### Example 4: Human 5-HT2A: Radioligand Binding Assay

### Objective

The objective of this study was to evaluate the binding properties of exemplary compounds of Formula I on 5-hydroxytryptamine receptor 2A (5-HT2A).

### 1 Materials and Instrumentation

1.1 Regents

| Items | Vendor | Cat# |
|---|---|---|
| Ketanserin Hydrochloride, | PerkinElmer | NET791250UC |
| [Ethylene-3H]- | | |
| Ketanserin | MedChemExpress | HY-10562 |
| Bovine Serum Albumin (BSA) | Sigma | A1933 |
| Calcium chloride (CaCl₂) | Sigma | C5670 |
| Tris(hydroxymethyl)aminomethane | Alfa Aesar | A18494 |
| (Tris) | | |
| Polyethylenimine, branched (PEI) | Sigma | 408727 |

1.2 Instrumentation and Consumables

| Item | Supplier | Cat# |
|---|---|---|
| Microbeta² Microplate Counter | PerkinElmer | 2450-0060 |
| UniFilter-96 GF/B | PerkinElmer | 6005177 |
| TopSeal | Biotss | SF-800 |
| MicroBeta Filtermate-96 | PerkinElmer | D961962 |
| Seven Compact pH meter | Mettler Toledo | S220 |
| Ultrapure Water Meter | Sichuan Ulupure | UPH-III-20T |
| Benchtop Centrifuge | Hunan Xiangyi | L550 |
| Microplate Shaker | Allsheng | MX100-4A |
| 384-Well Polypropylene Microplate | Labcyte | PP-0200 |
| 96 Round Well Plate | Corning | 3799 |
| 96 Round Deep Well Plate | Axygen | P-DW-11-C |
| Echo | LABCYTE | 550 |

### 2 Experimental Methods

1. The assay buffer was prepared following the table below.

| Reagent | Concentration | |
|---|---|---|
| Tris | 50 | mM |
| CaCl₂ | 4 | mM |
| BSA | 0.1% | (w/v) |

Adjust pH to 7.4 followed by 0.2 μM sterile filtration

2. 8 doses of reference and test compounds starting from 10 mM stock solution as required was prepared by 5-fold serial dilutions with 100% (v/v) DMSO.
3. UniFilter-96 GF/B plate was pretreated:
i. 50 μl/well of 0.5% (v/v) PEI was added to UniFilter-96 GF/C plates. The plates were sealed and incubated at 49C for 3 hrs.
ii. After incubation, the plates were washed 3 times with ice-cold wash buffer (50 mM Tris, pH7.4).
4. The assay plate was prepared:
i. Cell membrane was diluted with assay buffer and 330 μl/well was added to 96 round deep well plates to reach a concentration of 20 μg/well.
ii. 8 concentrations of reference or test compounds were prepared and 110 μl/well was added to 96 round deep well plates.
iii. [3H]-ketanserin was diluted with assay buffer to 5 nM (5×final concentration) and 110 μl/well was added to 96 round deep well plates.
5. The plate was centrifuged at 1000 rpm for 30 secs and then agitated at 600 rpm, R.T. for 5 min.
6. The plates were sealed and the plate incubated at 279C for 90 min.
7. The incubation was stopped by vacuum filtration onto GF/B filter plates followed by 4 times washing with ice-cold wash buffer (50 mM Tris, pH7.4).
8. The plates were dried at 379C for 45 min.
8. The filter plates were sealed and 40 μl/well of scintillation cocktail was added.
10. The plate was read by using a Microbeta² microplate counter.

### 3 Data Analysis

1. For reference and test compounds, the results were expressed as % Inhibition, using the normalization equation: N=100−100×(U−C2)/(C1−C2), where U is the unknown value, C1 is the average of high controls, and C2 is the average of low controls.
2. The IC₅₀ was determined by fitting percentage of inhibition as a function of compound concentrations with Hill equation using XLfit.

### Results and Discussion

The results of potential competition binding properties of the exemplary prodrug compounds, I-13 and I-22 of the application and their respective metabolites 10 and 13 targeting the human 5-hydroxytryptamine receptor 2A (5-HT2A) are summarized in Table 3. The results of exemplary compounds of the application are presented as IC₅₀ provided in Table 3.

TABLE 3
Effect of exemplary compounds of Formula I using Radioligand
binding assay on human 5-HT2A receptor

| Compound ID# | h5-HT2A, IC₅₀ [nM] |
|---|---|
| Psilocybin | 4248 |
| Psilocin | 187.2 |
| I-13 (Example 1) | 37.01 |
| 10 (Metabolite of I-13) | 218.8 |
| I-22 (Example 2) | 44.81 |
| 13 (Metabolite of I-22 | 219.2 |

### II. Results & Discussion

Exemplary compounds of Formula I were evaluated using radioligand binding assay on human 5-HT2A receptor. IC₅₀ (nM) concentrations are illustrated in Table 3. This assay confirms that precursor parent compounds or their respective metabolites of the application are effective ligands of the target human 5-HT2A receptors.

### Example 5: Human 5-HT1A: Functional FLIPR Assay

### 1 Objective

The potential excitatory effects of compounds targeting on 5-hydroxytryptamine receptor 1A (5-HT1A) under agonist mode was assessed.

### 2 Materials and Instrumentation

2.1 Cell line

| Cell line Name | Target | Host cell |
|---|---|---|
| HTR1A&Gα15-CHO | 5HT1A | Flp-in CHO |

2.2 Materials

| Regents | Vendor | Cat# |
|---|---|---|
| DMEM/F12 | Gibco | 11330057 |
| Dialyzed FBS | BIOSUN | BS-0005-500 |
| Penicillin-Streptomycin | Invitrogen | 15140 |
| Hygromycin B | Invivogen | Ant-hg-5 |
| TrypLE ™Express | Gibco | 12604-013 |
| DPBS | Gibco | 14190250 |
| DMSO | Millipore | 1029312500 |
| Probenecid | Sigma | P8761 |
| FLIPR Calcium 6 Assay Kit | Molecular Device | R8191 |
| HEPES | Invitrogen | 15630 |
| Hank's Buffered Saline Solution | Invitrogen | 14025 |
| Serotonin HCl | Selleck | S4244 |

2.3 Instrumentation and consumables

| Item | Supplier | Cat# |
|---|---|---|
| Fluorometric Imaging Plate Reader (FLIPR) | Molecular Device | Tetra |
| Countess Automated Cell Counter | Invitrogen | Countess |
| Cell Counting Chamber Slides | Invitrogen | C10312 |
| STERI-CYCLE CO2 Incubator | Thermo | 371 |
| 1300 Series Class II Biological Safety Cabinet | Thermo | 1389 |
| Table-type Large Capacity Low Speed Centrifuge | Cence | L550 |
| Centrifuge | Eppendorf | 5702 |
| Echo | Labcyte | 550 |
| Echo | Labcyte | 655 |
| Electro-thermal incubator | Shanghai Yiheng | DHP-9031 |
| plate shaker | IKA | MS3 digital |
| Water Purification System | ULUPURE | UPH-III-20T |
| Versatile and Universal pH and Conductivity Meters | Mettler Toledo | S220 |
| 384-Well plate | Corning | 3764 |
| 384-Well LDV Clear microplate | LABCYTE | LP-0200 |
| 384-Well Polypropylene microplate | LABCYTE | PP-0200 |
| 384-well compound plate | Corning | 3657 |
| T25 cell culture flask | Corning | 430639 |
| 50 mL Polypropylene Centrifuge Tube | JET | CFT011500 |
| 15 mL Polypropylene Centrifuge Tube | JET | CFT011150 |

### 3 Experimental Methods

### 3.1 Cell Culture

HTR1A&Gα15-CHO cells were cultured in DMEM/F12 medium containing 10% dialyzed FBS, 1×penicillin-streptomycin and 600 μg/mL Hygromycin B. The cells were passaged about three times a week, maintained between ~30% to ~90% confluence.

### 3.2 Cell Plating

1. The cell culture medium (DMEM/F12 medium containing 10% dialyzed FBS, 1×penicillin-streptomycin and 600 μg/mL Hygromycin B), TrypLE™ Express and DPBS was warmed to R.T. in advance.
2. The cell culture medium was removed from flask. Washed cells with DPBS.
3. 1 mL TrypLE™ Express was added to the flask, mixed well by gentle shaking and cells were incubated at 37° C. for a few minutes.
4. The cells were checked for morphological change under microscope, the digestion was stopped by adding 2 mL cell culture medium to the flask when most of cells turned to round.
5. The cell suspension was transferred into a 15 mL centrifuge tube, and then centrifuged at 1,200 rpm for 5 minutes.
6. The supernatant was removed. The cell pellet were resuspended with 2 mL cell culture medium.
7. The cell density were counted using cell counter. Only cells with >85% viability were used for the assay.
8. Cells were diluted to 4×10⁵/mL with cell culture medium.
9. 30 μL/well cell suspensions were added into a 384-well cell plate (The cell density was 12,000 cells/well).
10. The cell plate was incubated overnight at 37° C., 5% (v/v) CO₂.

### 3.3 Cell Handling

1. On the day of experiments, culture medium was removed from the cell plate.
2. 10 μL of assay buffer (20 mM HEPES, in 1×HBSS, pH 7.4) was added to each well of the cell plate.
3. 2×dye solution was prepared following the manufacture's instruction of the FLIPR® Calcium 6 assay kit:
i. The dye was diluted with assay buffer.
ii. probenecid was added to the final concentration of 5 mM.
iii. Vortexed vigorously for I-2 minutes, adjust pH to 7.4.
4. 10 μL of 2×dye solution was added to each well of the cell plate.
5. The cell plate was placed on plate shaker, followed by shaking at 600 rpm for 2 minutes.
6. The plate was incubated at 37° C. for 2 hours followed by an additional 15-minute incubation at 25° C.

### 3.4 Prepare 3×Compounds.

1. Serotonin was prepared to the concentration of 10 mM with DMSO, 3-folds serial dilutions were performed with DMSO.
2. Prepare the test compound to the concentration of 10 mM with DMSO, preform 3-folds serial dilutions with DMSO.
3. The compounds were added to a 384-well compound source plate.
4. 90 nL/well of serial diluted compounds were transferred from source plate to a 384-well compound plate by using an Echo.
5. 30 μL/well of assay buffer was added to the compound plate.
6. The plate was mixed on-plate shaker for 2 minutes.

### 3.5 FLIPR Assay

1. After the cells incubate with dye solution, the cell plate, compound plate containing 3×compounds and FLIPR tips were placed into FLIPR.
2. 10 μL of 3×compounds were transferred from the compound plate to the cell plate by FLIPR.
3. The plate was read for 160 sec with 1 sec interval to obtain the data of agonist mode.

### 4 Data Analysis

1. The normalized fluorescence reading (RFU) was calculated as shown below, wherein Fmax and Fmin stand for maximum and minimum of calcium signal during defined time window:

$$RFU = F max - F min$$

2. EC₅₀ was calculated by fitting RFU against log of compound concentrations with Hill equation using XLfit.

### Results & Discussion

The results of potential competition binding properties of the exemplary prodrug compound (I-13 and I-22) of the application and their respective metabolites (10 and 13) targeting the human 5-hydroxytryptamine receptor 1A (5-HT1A) are summarized in Table 4. The results of exemplary compounds of the application are presented as EC₅₀ provided in Table 4.

TABLE 4
Effect of exemplary compounds of Formula I using
FLIPR functional assay on human 5-HT1A receptor

| Compound ID# | h5-HT1A, EC₅₀ [nM] | RFU@10 μM ⁽¹⁾ |
|---|---|---|
| Psilocybin | ND⁽²⁾ | 143 |
| Psilocin | ND | 140 |
| I-13 (Example 1) | ND | 87 |
| 10 (Metabolite of I-13) | ND | 425 |
| I-22 (Example 2) | ND | 130 |
| 13 (Metabolite of I-22) | ND | 307 |

⁽¹⁾ Curve fitting with activation (%) @ 10 mM with RFU
⁽²⁾Not detected

Exemplary compounds of Formula I were evaluated using functional FLIPR assay on human 5-HT1A receptor. EC50 (nM) concentrations are illustrated in Table 4. This assay confirms that compounds or metabolites of the application have moderate functional activity at the target human 5-HT1A receptors.

### Example 6: Human 5-HT1A: Radioligand Binding Assay

### 1 Objective

The objective of this study was to evaluate the binding properties of test compounds on 5-hydroxytryptamine receptor 1A (5-HT1A).

### 2 Materials and Instrumentation

2.1 Regents

| Items | Vendor | Cat# |
|---|---|---|
| [3H]-8-Hydroxy-DPAT | PE | NET929250UC |
| Serotonin HCl | Selleck | S4244 |
| Bovine Serum Albumin (BSA) | Sigma | A1933 |
| Calcium chloride (CaCl₂) | Sigma | C5670 |
| MgCl₂ | Sigma | M1028 |
| Tris(hydroxymethyl)aminomethane (Tris) | Alfa Aesar | A18494 |
| Polyethylenimine, branched (PEI) | Sigma | 408727 |

2.2 Instrumentation and Consumables

| Item | Supplier | Cat# |
|---|---|---|
| Microbeta² Microplate Counter | PerkinElmer | 2450-0060 |
| UniFilter-96 GF/B | PerkinElmer | 6005177 |
| TopSeal | Biotss | SF-800 |
| MicroBeta Filtermate-96 | PerkinElmer | D961962 |
| Seven Compact pH meter | Mettler Toledo | S220 |
| Ultrapure Water Meter | Sichuan Ulupure | UPH-III-20T |
| Benchtop Centrifuge | Hunan Xiangyi | L550 |
| Microplate Shaker | Allsheng | MX100-4A |
| 384-Well Polypropylene Microplate | Labcyte | PP-0200 |
| 96 Round Well Plate | Corning | 3799 |
| 96 Round Deep Well Plate | Axygen | P-DW-11-C |
| Echo | LABCYTE | 550 |

### 3 Experimental Methods

1. The assay buffer was prepared following the table below.

| Reagent | Concentration | |
|---|---|---|
| Tris | 25 | mM |
| MgCl₂ | 10 | mM |
| CaCl₂ | 1 | mM |
| BSA | 0.5% | (w/v) |

Adjust pH to 7.4 followed by 0.2 μM sterile filtration

2. 8 doses of reference and test compounds were prepared starting from 10 mM stock solution as required by 5-fold serial dilutions with 100% (v/v) DMSO.
3. UniFilter-96 GF/B plate was pretreated:
i. 50 μl/well of 0.5% (v/v) PEI was added to UniFilter-96 GF/B plates. The plates were sealed and incubated at 4° C. for 3 hrs.
ii. After incubation, the plates were washed 3 times with ice-cold wash buffer (50 mM Tris, pH7.4).
4. Assay plate was prepared:
i. Cell membrane was diluted with assay buffer and 100 μl/well was added to 96 round well plates to reach a concentration of 20 μg/well.
ii. 8 concentrations of reference or test compounds were prepared and 50 μl/well was added to 96 round deep well plates.
iii. [3H]-8-Hydroxy-DPAT was diluted with assay buffer to 2 nM (4×final concentration) and 50 μl/well was added to 96 round well plates.
5. The plate was centrifuged at 1000 rpm for 30 secs and then agitated at 600 rpm, R.T. for 5 min.
6. The plates were sealed and the plate was incubated at 27° C. for 90 min.
7. The incubation was stopped by vacuum filtration onto GF/B filter plates followed by 4 times washing with ice-cold wash buffer (50 mM Tris, pH7.4).
8. The plates were dried at 37° C. for 45 min.
9. The filter plates were sealed and 40 μl/well of scintillation cocktail was added.
10. The plate was read by using a Microbeta² microplate counter.

### 4 Data Analysis

1. For reference and test compounds, the results were expressed as % Inhibition, using the normalization equation: N=100−100×(U−C₂)/(C₁−C₂), where U is the unknown value, C₁ is the average of high controls, and C₂ is the average of low controls.
2. The IC₅₀ was determined by fitting percentage of inhibition as a binding of compound concentrations with Hill equation using XLfit.

### Results and Discussion

The results of potential competition binding properties of the exemplary prodrug compounds, I-13 and I-22 of the application and their respective metabolites 10 and 13 targeting the human 5-hydroxytryptamine receptor (5-HT1A) are summarized in Table 5. The results of exemplary compounds of the application are presented as IC₅₀provided in Table 5.

TABLE 5
Effect of exemplary compounds of Formula I using Radioligand
binding assay on human 5-HT1A receptor

| Compound ID# | h5-HT1A, IC₅₀ [nM] |
|---|---|
| Psilocybin | 4785 |
| Psilocin | 195.7 |
| I-13 (Example 1) | 153.6 |
| 10 (Metabolite of I-13) | 148 |
| I-22 (Example 2) | 123.9 |
| 13 (Metabolite of I-22) | 156 |

### Results & Discussion

Exemplary compounds of Formula I and metabolites thereof were evaluated using radioligand binding assay on human 5-HT1A receptor. IC₅₀ (nM) concentrations are illustrated in Table 5. This assay confirms that precursor parent compounds or their respective metabolites of the application are effective ligands of the target human 5-HT1A receptors.

### Example 7: Human, Rat and Mouse Liver Microsomes Stability

### Objective

The objective of this study was to estimate in vitro metabolic stability of exemplary compounds of the application in pooled human, male rat and male mouse liver microsomes. The concentrations of parent compounds in reaction systems were evaluated by LC-MS/MS for estimating the stability in pooled human, male rat and male mouse liver microsomes. The in vitro intrinsic clearances of test compounds were determined as well.

### Protocol

A master solution in the “Incubation Plate” containing phosphate buffer, ultra-pure H₂O, MgCl₂ solution and liver microsomes was made according to Table 6. The mixture was pre-warmed at 37° C. water bath for 5 minutes.

TABLE 6
Preparation of master solution

| Reagent | Stock Concentration | | Volume | Final Concentration | |
|---|---|---|---|---|---|
| Phosphate buffer | 200 | mM | 200 μL | 100 | mM |
| Ultra-pure H₂O | — | | 106 μL | — | |
| MgCl₂ solution | 50 | mM | 40 μL | 5 | mM |
| Microsomes | 20 | mg/mL | 10 μL | 0.5 | mg/mL |

40 μL of 10 mM NADPH solution was added to each well. The final concentration of NADPH was 1 mM. The negative control samples were prepared by replacing NADPH with 40 μL of ultra-pure H₂O. Samples were prepared in duplicate. Negative controls were prepared in singlet.

The reaction was started with the addition of 4 μL of 200 μM exemplary test compounds of the application or control compounds to each master solution to get the final concentration of 2 μM. This study was performed in duplicate.

Aliquots of 50 μL were taken from the reaction solution at 0, 15, 30, 45 and 60 minutes. The reaction solutions were stopped by the addition of 4 volumes of cold methanol with IS (100 nM alprazolam, 200 nM imipramine, 200 nM labetalol and 2 μM ketoprofen). Samples were centrifuged at 3,220 g for 40 minutes. Aliquot of 90 μL of the supernatant was mixed with 90 μL of ultra-pure H2O and then was used for LC-MS/MS analysis.

LC/MS analysis was performed for all samples from this study using a Shimadzu liquid chromatograph separation system equipped with degasser DGU-20A5R; solvent delivery unit LC-30AD; system controller SIL-30AC; column oven CTO-30A; CTC Analytics HTC PAL System. Mass spectrometric analysis was performed using a Triple Quad™ 5500 instrument.

All calculations were carried out using Microsoft Excel. Peak area ratios of test compound to internal standard (listed in the below table) were determined from extracted ion chromatograms.

All calculations were carried out using Microsoft Excel. Peak areas were determined from extracted ion chromatograms. The slope value, k, was determined by linear regression of the natural logarithm of the remaining percentage of the parent drug vs. incubation time curve.

The in vitro half-life (in vitro t½) was determined from the slope value:

$$in vitro {t}_{1 / 2} = - \{ 0.693 / k \}$$

Conversion of the in vitro t½ (min) into the in vitro intrinsic clearance (in vitro CLint, in μL/min/mg proteins) was done using the following equation (mean of duplicate determinations):

$$in vitro {CL}_{int} = ( \frac{0.693}{( {t}_{1 / 2} )} ) * ( \frac{volume of incubation ( μL )}{amount of proteins ( mg )} )$$

For the exemplary compounds of the application or control compound that showed an initial fast disappearance followed by a slow disappearance, only the time points that were within the initial rate were included in the calculation.

### Results & Discussion

Human, rat and mouse liver microsomes contain a wide variety of drug metabolizing enzymes and are commonly used to support in vitro ADME (absorption, distribution, metabolism and excretion) studies. These microsomes are used to examine the potential first-pass metabolism by-products of orally administered drugs. Exemplary compounds of the application were evaluated for their stability in human, rat and mouse liver microsomes. A majority of the exemplary compounds of the application in three species, human, rat and mouse liver microsomes were recovered within a 60-minute time period indicating that the compounds were not rapidly cleared (see Table 7 for Exemplary compounds of Formula I).

TABLE 7
Metabolic stability of exemplary dimer compounds of Formula
I (I-13 and I-22) and control compounds diclofenac and
psilocin in human, rat and mouse with NADPH

| | t_{1/2} | | | CLᵢₙₜ | | |
|---|---|---|---|---|---|---|
| Example | (min) | | | (μL/min/mg protein) | | |
| ID# | Human | Rat | Mouse | Human | Rat | Mouse |
| Diclofenac | 10.35 | 15.73 | 40.63 | 133.92 | 88.12 | 34.11 |
| Psilocin | 117.32 | 89.01 | 141.71 | 11.81 | 15.57 | 9.78 |
| Example 1 | <4.51 | <4.51 | <4.51 | >307.01 | >307.01 | >307.01 |
| (I-13) | | | | | | |
| Example 2 | 4.67 | <4.51 | <4.51 | 296.69 | >307.01 | >307.01 |
| (I-22) | | | | | | |

*If % remaining at 30 minutes was lower than 1%, then CLᵢₙₜ and t1/2 reported as “>307.01” and “<4.51”, respectively.

TABLE 8
Metabolic stability of exemplary dimer compounds of Formula
I (I-13 and I-22) and control compounds diclofenac and
psilocin in human, rat and mouse liver microsomes

| | Remaining Percentage (%) | | | | | |
|---|---|---|---|---|---|---|
| | after 60 min | | | | | |
| | Human | | Rat | | Mouse | |
| Example | With | Without | With | Without | With | Without |
| ID# | cofactors | cofactors | cofactors | cofactors | cofactors | cofactors |
| Diclofenac | 1.90 | 95.90 | 7.11 | 97.86 | 35.94 | 97.86 |
| Psilocin | 70.16 | NT⁽¹⁾ | 62.68 | NT | 96.89 | NT |
| Example 1 | 0 | 0 | 0 | 0 | 0 | 0 |
| (I-13) | | | | | | |
| Example 2 | 0 | 1.38 | 0 | 0.22 | 0 | 0 |
| (I-22) | | | | | | |

⁽¹⁾Not tested

### Discussion

The results demonstrate the exemplary compounds (I-13 and I-22) are rapidly metabolized.

### Example 8: Human, Rat, Mouse and Dog: Plasma Stability

### 1. Preparation of Stock Solutions

The stock solution of test compound was prepared in DMSO and diluted at the final concentration of 200 μM. 1 mM lovastatin and propantheline working solution was pre-pared in DMSO and acetonitrile, respectively. Lovastatin was used as positive control for rat and dog plasma stability assay. Propantheline was used as positive control in human, mouse and monkey plasma stability assay.

### 2. Procedures for Plasma Stability

a. 2.5 μL of 200 μM or 1 mM test compound or control compound solution was spiked to 497.5 μL plasma to reach a final concentration of 1 μM or 5 μM. The final concentration of organic solvents was 0.5%. The assay was performed in duplicated.
b. The reaction samples were incubated at 37° C. at approximately 60 rpm in a water bath.
c. Aliquots of 50 μL were taken from the reaction samples at 0, 30, 60, 120, 180 and 240 minutes. The reaction was stopped by the addition of 7 volumes of cold acetonitrile containing internal standards (IS: 100 nM alprazolam, 200 nM imipramine, 200 nM labetalol and 2 μM ketoprofen).
d. All samples were vortexed for 2 minutes, followed by centrifugation at 3,220 g for 30 minutes to precipitate proteins. 100 μL of the supernatant was transferred to a new plate. The supernatant was diluted with ultrapure water according to the LC-MS signal response and peak shape.

### 3. Sample Analysis

Samples were analyzed by LC-MS/MS.
LC system: Shimadzu
MS analysis: Triple Quad™ 6500+ from AB Inc (Canada) with an ESI interface
Column temperature: 40° C.
Column: Xselect® Hss T3 2.5μ (2.1×30 mm) coupled with preguard column
Mobile phase: 0.1% formic acid in water (A) and 0.1% formic acid in acetonitrile (B)

| Time (min) | 0 | 0.1 | 0.5 | 0.8 | 0.81 | 1.0 |
|---|---|---|---|---|---|---|
| % B | 5 | 5 | 100 | 100 | 5 | 5 |

### 4. Data Analysis

All calculations were carried out using Microsoft Excel. Remaining percentages of parent compounds at each time point were estimated by determining the peak area ratios from extracted ion chromatograms.

TABLE 9
Stability Results of Exemplary Compounds in Plasma of Different Species

| | | T_{1/2} | Remaining Percentage (%) | | | | | |
|---|---|---|---|---|---|---|---|---|
| Compound | Species | (min) | 0 min | 30 min | 60 min | 120 min | 180 min | 240 min |
| Propantheline | Human | 9.53 | 100.00 | 18.68 | 1.28 | 0.00 | 0.00 | 0.00 |
| I-13 | | N.A. | 100.00 | 0.00 | 0.00 | 0.00 | 0.00 | 0.00 |
| I-22 | | N.A. | 100.00 | 0.00 | 0.00 | 0.00 | 0.00 | 0.00 |
| Lovastatin | Rat | N.A. | 100.00 | 0.00 | 0.00 | 0.00 | 0.00 | 0.00 |
| I-13 | | N.A. | 100.00 | 0.00 | 0.00 | 0.00 | 0.00 | 0.00 |
| I-22 | | N.A. | 100.00 | 0.00 | 0.00 | 0.00 | 0.00 | 0.00 |
| Propantheline | Mouse | 34.36 | 100.00 | 65.32 | 32.49 | 9.12 | 2.90 | 0.87 |
| I-13 | | N.A. | 100.00 | 0.00 | 0.00 | 0.00 | 0.00 | 0.00 |
| I-22 | | N.A. | 100.00 | 0.00 | 0.00 | 0.00 | 0.00 | 0.00 |
| Lovastatin | Dog | 73.25 | 100.00 | 100.99 | 64.77 | 40.82 | 22.40 | 11.03 |
| I-13 | | 49.20 | 100.00 | 67.21 | 44.90 | 15.17 | 8.32 | 3.48 |
| I-22 | | 24.89 | 100.00 | 39.05 | 14.86 | 3.51 | 0.92 | 0.56 |

1. If no peak detected at 30 min, then T_{1/2} was reported as “N.A.”.
2. For the compounds that showed an initial fast disappearance followed by a slow disappearance, only the time points that were within the initial rate were included in the calculation.

### Example 9: Intestinal Mucosal Permeation of Exemplary Compounds of the Application and Metabolites Thereof) Using Caco-2 Cell Monolayers

### 1. Preparation for Cell Seeding

1) Caco-2 cell culture medium was prepared consisting of Dulbecco's Modified Eagle's Medium (DMEM) with high glucose and L-glutamine supplemented with 10% FBS, 1×penicillin-streptomycin mixture and 1×non-essential amino acids (NEAA).
2) 50 μL of culture medium was added to each well of the Transwell insert. Transwell insert was removed from reservoir and 25 mL of culture medium was added.
3) Incubated at 37° C., 5% CO₂ for 1 hour. Plates were ready for cell seeding.
4) Cells were cultivated in T-75 flasks in a cell culture incubator set at 37° C., 5% CO₂, 95% relative humidity. Cells were allowed to reach 80-90% confluence before detaching and splitting.
5) Cultivated cells were rinsed in T-75 flasks with 5 mL PBS. Cells were aspirated off, then 1.5 mL trypsin/EDTA was added, and incubated at 37° C. for approximately 5 to 10 minutes or until the cells detach and float. Trypsin/EDTA was inactivated by adding excess serum containing medium.
6) Cell suspension was removed to a conical tube and cells were pelleted by centrifugation at 120×g for 10 minutes.
7) Cells were resuspended in seeding medium at a density of 6.86×10⁵ cells/mL. This cell concentration was used to seed 2.40×10⁵ cells/cm².

### 2. Seeding and Feeding of Caco-2 Cells in Transwell Plates

1) 50 μL of above cell suspension was added to each well of a previously prepared Transwell plate.
2) The plate was incubated for 14-18 days. The medium was replaced every other day, beginning no sooner than 48 hours after initial plating.
3) The procedure for medium changes was as follows: The plate was removed from incubator and placed in hood. The medium was aspirated from reservoir and each Transwell insert. 75 μL of culture medium was added to each well of Transwell inserts and 25 mL of culture medium was added to reservoir tray. Plate was returned to incubator.

### 3. Assessment of Cell Monolayer Integrity

1) When the 14-day Caco-2 cultured cells reached confluence and are differentiated, they were ready to be used for transport studies.
2) Medium was removed from reservoir and Transwell inserts.
3) 75 μL of pre-warmed culture medium was added to each transwell insert and 25 mL of reservoir tray.
4) The electrical resistance across the monolayer was measured using automated tissue resistance measuring system (World Precision Instruments, Sarasota, FL).
5) The electrical resistance was recorded for each well.
6) Once all wells were measured, plate was returned to incubator.
7) TEER of each well was calculated by the equation below. The TEER value of each well should be greater than 230 ohms·cm².

TEER measurement (ohms)×Area of membrane (cm²)=TEER value (ohm·cm²)

### 4. Performing the Drug Transport Assay

1) The Caco-2 plate was removed from the incubator. Next, the monolayer was washed, the volume was exchanged two times using pre-warmed HBSS (10 mM HEPES, pH 7.4). Then the plate was incubated for 30 minutes at 37° C.
2) 1 mM stock solutions of control compounds and test compound(s) were prepared in DMSO and diluted with HBSS (10 mM HEPES, pH 7.4) to reach the final concentration of 5 μM. The final concentration of DMSO in the incubation system was 0.5%. Digoxin, prazosin and propranolol were used as control compounds in this assay.
3) HBSS (10 mM HEPES, pH 7.4) was removed after 30 minutes pre-incubation.
4) To determine the rate of drug transport in the apical to basolateral direction: 75 μL of the control compounds and test compound were added to the Transwell insert (apical compartment). The wells in the receiver plate (basolateral compartment) were filled with 235 μL of HBSS (10 mM HEPES, pH 7.4).
5) To determine the rate of drug transport in the basolateral to apical direction: 235 μL of the control compounds and test compound(s) was added to the receiver plate wells (basolateral compartment). The Transwell insert (apical compartment) was filled with 75 μL of HBSS (10 mM HEPES, pH 7.4).
6) Time 0 samples were prepared by transferring 50 μL of working solution to wells of the 96-deepwell plate, followed by adding 200 μL of cold methanol containing appropriate internal standards (100 nM alprazolam, 200 nM labetalol, 200 nM caffeine and 200 nM diclofenac).
7) Incubated at 37° C. for 2 hours.
8) At the end of the transport period, 50 μL samples were removed from donor sides and receiver sides and transferred to a new pate. Then 200 μL of cold methanol was added containing internal standards (100 nM alprazolam, 200 nM labetalol, 200 nM caffeine and 200 nM diclofenac) to terminate the reaction. Vortexed for 5 minutes. Samples were centrifuged at 3,220 g for 40 minutes. An aliquot of 100 μL of the supernatant was mixed with 100 μL of ultra-pure water for LC-MS/MS analysis. All incubations were performed in duplicate.
9) The solution from transwell plate was discarded. 100 μL Lucifer Yellow solution (100 μM in HBSS) was added to each well of transwell insert and 300 μL of HBSS was added to each well of receiver. Incubated at 37° C. for 30 minutes. 80 μL was removed from each well of apical and basolateral sides to a solid black plate. The plate was read with Tecan Infinite™ M 200 (Excitation/Emission wavelength 485 nM/530 nM).

### Results & Discussion

The P_{app (A→B)} and P_{app (B→A)} values for compound 10 (metabolite of I-13) were 23.20 and 18.75 cm/s×10⁻⁶, respectively, with corresponding efflux ratios of 0.81. The permeability results for compound 10 in Caco-2 cell monolayers are presented in Table 10.

The results indicate that exemplary dimer compounds of Formula I and their respective metabolites have high permeability compounds and are unlikely substrates of efflux transporters.

TABLE 10
Permeability results for representative compounds
of Formula I and their respective metabolites
in Caco-2 cell monolayers (average, n = 2)

| | Pₐₚₚ | | Percentage | | |
|---|---|---|---|---|---|
| | (cm/s × 10⁻⁶) | | recovery (%) | | Efflux |
| Test Compound | A→B | B→A | A→B | B→A | ratio |
| *Propranolol | 33.5 | 23.09 | 91.34 | 92.59 | 0.69 |
| *Digoxin | 0.53 | 18.89 | 86.58 | 104.27 | 35.38 |
| *Prazosin | 10.14 | 33.06 | 79.47 | 104.81 | 3.28 |
| Psilocybin | 0.06 | 0.03 | 41.39 | 103.77 | 0.45 |
| Psilocin | 24.57 | 18.71 | 100.30 | 102.03 | 0.76 |
| Example 1 (I-13) | 0.63 | 4.10 | 94.72 | 109.81 | 6.50 |
| Compound 10 | 23.20 | 18.75 | 82.74 | 92.04 | 0.81 |
| Example 2 (I-22) | <0.25 | 1.01 | <125.72 | 100.86 | >4.08 |
| **compound 13 | NT | NT | NT | NT | NT |

*values represents average of n = 3
**not tested

TABLE 11
Assessment of Caco-2 Cell Monolayer Integrity

| | TEER_{A-B} | TEER_{B-A} | LY Leakage_{A-B} | LY Leakage_{B-A} |
|---|---|---|---|---|
| Compound ID | (W × cm²) | (W × cm²) | (%) | (%) |
| *Propranolol | 807 | 835 | 0.50 | 0.43 |
| *Digoxin | 817 | 851 | 0.53 | 0.46 |
| *Prazosin | 815 | 841 | 0.39 | 0.41 |
| I-22 | 738 | 740 | 0.71 | 0.31 |
| I-13 | 769 | 809 | 0.33 | 0.46 |

*values represents average of n = 3

While the present application has been described with reference to examples, it is to be understood that the scope of the claims should not be limited by the embodiments set forth in the examples but should be given the broadest interpretation consistent with the description as a whole.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the application described and claimed herein.

1. A compound of Formula I: 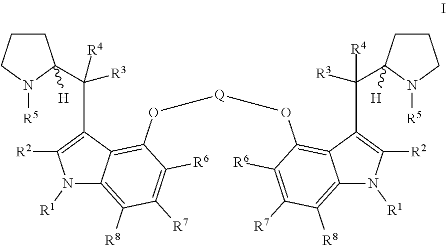
or a pharmaceutically acceptable salt, solvate, and/or prodrug thereof,
wherein:
Q is selected from P(O)OR⁹, C₁-C₄alkylene-P(O)OR⁹—C₁-C₆alkylene, C(O), S(O)₂, C(O)Q′C(O), C(O)OQ′OC(O), and C(O)NR^{9′}Q′NR^{9′}C(O);
R¹ is selected from H, C₁-C₃alkyl, C(O)R¹⁰, CO₂R¹⁰, C(O)N(R¹⁰)(R¹¹), S(O)R¹⁰, and SO₂R¹⁰;
R², R³, R⁴, and R⁵are independently selected from H and C₁-C₆alkyl;
R⁶, R⁷, and R⁸ are independently selected from H, halo, CN, OR¹², N(R¹²)(R¹³), SR¹², C₁-C₆alkyl, C₁-C₆haloalkyl, C₂-C₆haloalkenyl, CO₂R¹², C(O)N(R¹²)(R¹³), S(O)R¹², SO₂R¹², C₂-C₆alkenyl, C₂-C₆alkynyl, C₂-C₆haloalkynyl, C₃-C₇cycloalkyl, and C₃-C₇heterocycloalkyl comprising 1 to 2 heteromoieties selected from O, S, S(O), SO₂, N, and NR¹⁴, wherein said C₁-C₆alkyl, C₁-C₆haloalkyl, C₂-C₆alkenyl, C₂-C₆haloalkenyl, C₂-C₆alkynyl, C₂-C₆haloalkynyl, C₃-C₇cycloalkyl, and C₃-C₇heterocycloalkyl are optionally substituted by one or more substituents independently selected from CN, OR¹⁵, N(R¹⁵)(R¹⁶), and SR¹⁵, and wherein said C₃-C₇cycloalkyl and C₃-C₇heterocycloalkyl are each further optionally substituted with one or more substituents selected from halo, CO₂R¹⁷, C(O)N(R¹⁷)(R¹⁸), SO₂R¹⁷, C₁-C₆alkyl, C₁-C₆haloalkyl, C₂-C₆alkenyl, C₂-C₆haloalkenyl, C₂-C₆alkynyl, C₂-C₆haloalkynyl, C₃-C₆cycloalkyl, and C₃-C₆heterocycloalkyl comprising 1 to 2 heteromoieties selected from O, S, S(O), SO₂, N, and NR¹⁹;
Q′ is selected from a direct bond, C₁-C₂₀alkylene, C₁-C₂₀haloalkylene, C₂-C₂₀alkenylene, C₂-C₂₀haloalkenylene, C₂-C₂₀alkynylene, C₂-C₂₀haloalkynylene, C₃-C₇cycloalkylene, and C₃-C₇heterocycloalkylene comprising 1 to 2 heteromoieties selected from O, S, S(O), SO₂, N, and NR²⁰, wherein said C₁-C₂₀alkylene, C₂-C₂₀haloalkylene, C₂-C₆alkenylene, C₂-C₂₀haloalkenylene, C₃-C₇cycloalkylene, and C₃-C₇heterocycloalkylene are optionally substituted with one or more substituents independently selected from CN, OR²¹, N(R²¹)(R²²), and SR²¹, and/or are disubstituted on the same carbon atom with C₁₋₆alkyl, or with C₂₋₆alkylene to form a C₃-C₇cycloalkyl ring, and wherein said C₃-C₇cycloalkylene and C₃-C₇heterocycloalkylene are each further optionally substituted with one or more substituents selected from C₁-C₃alkyl and C₁-C₃haloalkyl, provided when Q is C(O)OQ′OC(O) or C(O)NR^{9′}Q′NR^{9′}C(O), then Q′ is not a direct bond;
each R⁹, R¹⁰, R¹¹, R¹², R¹³, R¹⁴, R¹⁵, R¹⁶, R¹⁷, R¹⁸ R¹⁹, R²⁰, R²¹, and R²² is independently selected from H, substituted or unsubstituted C₁-C₆alkyl, substituted or unsubstituted C₂-C₆alkenyl, substituted or unsubstituted C₂-C₆alkynyl, substituted or unsubstituted C₁-C₅haloalkyl, substituted or unsubstituted C₃-C₇cycloalkyl, substituted or unsubstituted C₃-C₇heterocycloalkyl, substituted or unsubstituted aryl, substituted or unsubstituted heteroaryl, substituted or unsubstituted C₁-C₆alkyleneC₃-C₇cycloalkyl, substituted or unsubstituted C₁-C₆alkyleneC₃-C₇heterocycloalkyl, substituted or unsubstituted C₁-C₆alkylenearyl, and substituted or unsubstituted C₁-C₆alkyleneheteroaryl; and
R^{9′} is independently selected from H and C₁-C₆alkyl;
wherein all available hydrogen atoms are optionally substituted with a fluorine atom or a chlorine atom and/or all available atoms are optionally substituted with an alternate isotope thereof.

2. The compound of claim 1, wherein R¹ is selected from H, C₁-C₃alkyl, C(O)R¹⁰, CO₂R¹⁰, and C(O)N(R¹⁰)(R¹¹), wherein all available hydrogen atoms are optionally substituted with a fluorine atom or a chlorine atom and/or all available atoms are optionally substituted with an alternate isotope thereof.

3. The compound of claim 2, wherein R¹ is selected from is selected from H, CH₃, and CH₂CH₃, wherein all available hydrogen atoms are optionally substituted with a fluorine atom or a chlorine atom and/or all available atoms are optionally substituted with an alternate isotope thereof.

4. (canceled)

5. (canceled)

6. The compound of claim 1, wherein R², R³, and R⁴ are independently selected from H, CH₃, CH₂CH₃, CH(CH₃)₂, and C(CH₃)₃, wherein all available hydrogen atoms are optionally substituted with a fluorine atom or a chlorine atom and/or all available atoms are optionally substituted with an alternate isotope thereof.

7. (canceled)

8. (canceled)

9. (canceled)

10. (canceled)

11. The compound of claim 1, wherein R⁵ is selected from H, D, F, CH₃, CD₂H, CDH₂, CD₃, CF₃, CHF₂, CFH₂, CH₂CH₃, CH₂CH₂D, CH₂CD₂H, and CD₂CD₃.

12. (canceled)

13. The compound of claim 1, wherein R⁶, R⁷, and R³ are independently selected from H, F, Cl, Br, CN, OR¹², N(R¹²)(R¹³), SR¹², C₁-C₄alkyl, C₁-C₄haloalkyl, C₂-C₆haloalkenyl, CO₂R¹², C(O)N(R¹²)[[ ]](R¹³), S(O)R¹², SO₂R¹², C₂-C₆alkenyl, C₂-C₆alkynyl, and C₂-C₆haloalkynyl, wherein said C₁-C₄alkyl, C₁-C₄haloalkyl, C₂-C₆alkenyl, C₂-C₆haloalkenyl, C₂-C₆alkynyl, and C₂-C₆haloalkynyl groups are optionally substituted by one to three substituents independently selected from CN, OR¹⁵, N(R¹⁵)(R¹⁶), and SR¹⁵, wherein all available hydrogen atoms are optionally substituted with a fluorine atom or chlorine atom and/or all available atoms are optionally substituted with an alternate isotope thereof.

14. The compound of claim 13, wherein R⁶, R⁷, and R³ are independently selected from H, D, F, Cl, Br, and CN.

15. The compound of claim 1, wherein Q is selected from P(O)OR⁹ and C₁-C₂alkylene-P(O)OR⁹—C₁-C₂alkylene, wherein all available hydrogen atoms are optionally substituted with a fluorine atom or a chlorine atom and/or all available atoms are optionally substituted with an alternate isotope thereof.

16. The compound of claim 15, wherein Q is CH₂—P(O)OR⁹—CH₂, wherein all available hydrogen atoms are optionally substituted with a fluorine atom or a chlorine atom and/or all available atoms are optionally substituted with an alternate isotope thereof.

17. The compound of claim 1, wherein Q is selected from C(O), C(O)Q′C(O), C(O)OQ′OC(O), and C(O)NR^{9′}Q′NR^{9′}C(O), wherein all available hydrogen atoms are optionally substituted with a fluorine atom or a chlorine atom and/or all available atoms are optionally substituted with an alternate isotope thereof.

18. The compound of claim 17, wherein Q is selected from C(O)Q′C(O), C(O)OQ′OC(O), and C(O)NR^{9′}Q′NR⁹C(O), wherein Q′ is selected from C₁-C₁₀alkylene, C₂-C₁₀alkenylene, and C₂-C₁₀alkynylene, wherein said C₁-C₁₀alkylene, C₂-C₁₀alkenylene, and C₂-C₁₀alkynylene are optionally substituted by one to three substituents independently selected from CN, OR²¹, N(R²¹)(R²²), and SR²¹, and/or disubstituted on the same carbon atom with C₁₋₆alkyl, or with C₂₋₆alkylene to form a C₃-C₇cycloalkyl ring, wherein said C₃-C₇cycloalkyl is further optionally substituted with a substituent selected from C₁-C₃alkyl and C₁-C₃haloalkyl, wherein all available hydrogen atoms are optionally substituted with a fluorine atom or a chlorine atom and/or all available atoms are optionally substituted with an alternate isotope thereof.

19. The compound of claim 18, wherein Q′ is selected from C₁-C₆alkylene, C₂-C₆alkenylene and C₂-C₆alkynylene optionally substituted with one or two substituents independently selected from OR²¹ and N(R²¹)(R²²), and/or disubstituted on the same carbon atom with C₁₋₆alkyl, or with C₂₋₆alkylene to form a C₃-C₇cycloalkyl ring, wherein said C₃-C₇cycloalkyl ring is further optionally substituted with a substituent selected from C₁-C₃alkyl and C₁-C₃haloalkyl, wherein all available hydrogen atoms are optionally substituted with a fluorine atom or a chlorine atom and/or all available atoms are optionally substituted with an alternate isotope thereof.

20. The compound of claim 19, wherein Q′ is selected from C₁-C₄alkylene and C₂-C₄alkenylene, wherein all available hydrogen atoms are optionally substituted with a fluorine atom or a chlorine atom and/or all available atoms are optionally substituted with an alternate isotope thereof.

21. The compound of claim 17, wherein Q is selected from C(O)Q′C(O), C(O)OQ′OC(O), and C(O)NR^{9′}Q′NR^{9′}C(O), and wherein Q′ is a direct bond.

22. The compound of claim 1, wherein R⁹, R¹⁰, R¹¹, R¹², R¹³, R¹⁴, R¹⁵, R¹⁶, R¹⁷, R¹⁸, R¹⁹, R²⁰, R²¹, and R²²are independently selected from H, substituted or unsubstituted C₁-C₄alkyl, substituted or unsubstituted C₂-C₆alkenyl, substituted or unsubstituted C₂-C₆alkynyl, substituted or unsubstituted C₁-C₄haloalkyl, substituted or unsubstituted C₃-C₇cycloalkyl, substituted or unsubstituted heterocycloalkyl, substituted or unsubstituted aryl, and substituted or unsubstituted heteroaryl, wherein all available hydrogen atoms are optionally substituted with a fluorine atom or a chlorine atom and/or all available atoms are optionally substituted with an alternate isotope thereof.

23. The compound of claim 22, wherein R⁹, R¹⁰, R¹¹, R¹², R¹³, R¹⁴, R¹⁵, R¹⁶, R¹⁷, R¹⁸, R¹⁹, R²⁰, R²¹, and R²² are independently selected from H, substituted or unsubstituted C₁-C₄alkyl, substituted or unsubstituted C₂-C₆alkenyl, substituted or unsubstituted C₂-C₆alkynyl, and substituted or unsubstituted C₁-C₄haloalkyl, wherein all available hydrogen atoms are optionally substituted with a fluorine atom or a chlorine atom and/or all available atoms are optionally substituted with an alternate isotope thereof.

24. The compound of claim 23, wherein R⁹, R¹⁰, R¹¹, R¹², R¹³, R¹⁴, R¹⁵, R¹⁶, R¹⁷, R¹⁸, R¹⁹, R²⁰, R²¹, and R²² are independently selected from H, D, CH₃, CD₂H, CDH₂, CD₃, CF₃, CHF₂, CF₂H, CH₂CH₂D, CH₂CD₂H, CH₂CH₃, and CD₂CD₃.

25. (canceled)

26. The compound of claim 1, selected from:

| Compound ID | Chemical Structure |
|---|---|
| 1-1 | 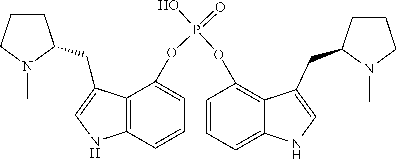 [SMILES: CN1CCC[C@@H]1Cc1c[nH]c2cccc(OP(=O)(O)Oc3cccc4[nH]cc(C[C@H]5CCCN5C)c34)c12] |
| 1-2 | 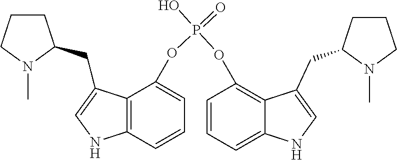 [SMILES: CN1CCC[C@H]1Cc1c[nH]c2cccc(OP(=O)(O)Oc3cccc4[nH]cc(C[C@@H]5CCCN5C)c34)c12] |
| 1-3 | 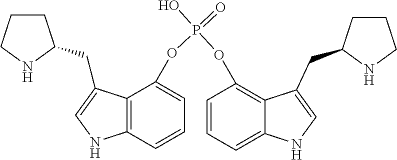 [SMILES: O=P(O)(Oc1cccc2[nH]cc(C[C@H]3CCCN3)c12)Oc1cccc2[nH]cc(C[C@H]3CCCN3)c12] |
| 1-4 | 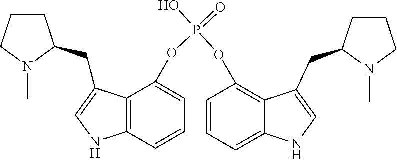 [SMILES: CN1CCC[C@@H]1Cc1c[nH]c2cccc(OP(=O)(O)Oc3cccc4[nH]cc(C[C@@H]5CCCN5C)c34)c12] |
| 1-5 | 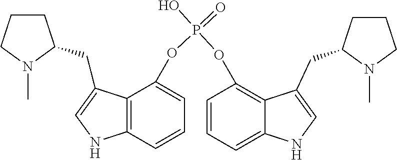 [SMILES: CN1CCC[C@@H]1Cc1c[nH]c2cccc(OP(=O)(O)Oc3cccc4[nH]cc(C[C@@H]5CCCN5C)c34)c12] |
| 1-6 | 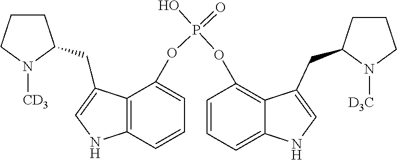 [SMILES: [2H]C([2H])([2H])N1CCC[C@@H]1Cc1c[nH]c2cccc(OP(=O)(O)Oc3cccc4[nH]cc(C[C@H]5CCCN5C([2H])([2H])[2H])c34)c12] |
| 1-7 | 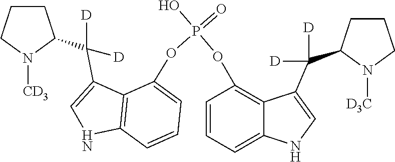 [SMILES: [2H]C([2H])(c1c[nH]c2cccc(OP(=O)(O)Oc3cccc4[nH]cc(C([2H])([2H])[C@H]5CCCN5C([2H])([2H])[2H])c34)c12)[C@H]1CCCN1C([2H])([2H])[2H]] |
| 1-8 | 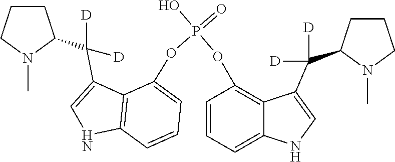 [SMILES: [2H]C([2H])(c1c[nH]c2cccc(OP(=O)(O)Oc3cccc4[nH]cc(C([2H])([2H])[C@H]5CCCN5C)c34)c12)[C@H]1CCCN1C] |
| 1-9 | 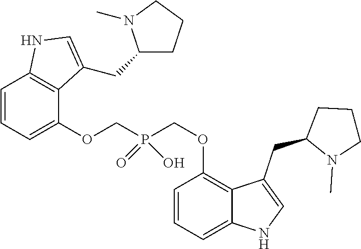 [SMILES: CN1CCC[C@@H]1Cc1c[nH]c2cccc(OCP(=O)(O)COc3cccc4[nH]cc(C[C@H]5CCCN5C)c34)c12] |
| 1-10 | 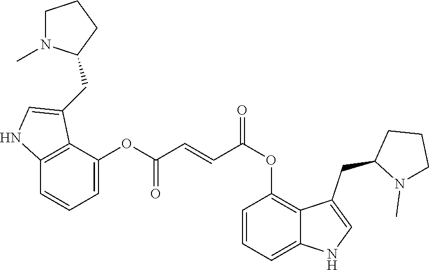 [SMILES: CN1CCC[C@@H]1Cc1c[nH]c2cccc(OC(=O)/C=C/C(=O)Oc3cccc4[nH]cc(C[C@H]5CCCN5C)c34)c12] |
| 1-11 | 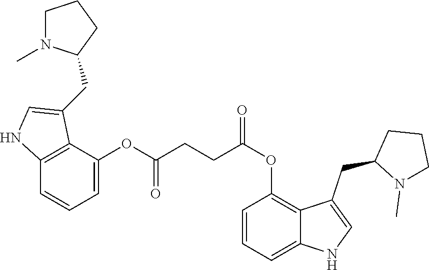 [SMILES: CN1CCC[C@@H]1Cc1c[nH]c2cccc(OC(=O)CCC(=O)Oc3cccc4[nH]cc(C[C@H]5CCCN5C)c34)c12] |
| 1-12 | 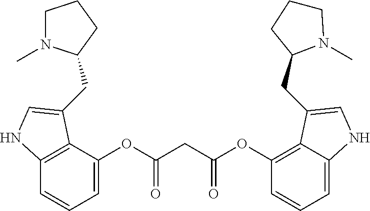 [SMILES: CN1CCC[C@@H]1Cc1c[nH]c2cccc(OC(=O)CC(=O)Oc3cccc4[nH]cc(C[C@H]5CCCN5C)c34)c12] |
| 1-13 | 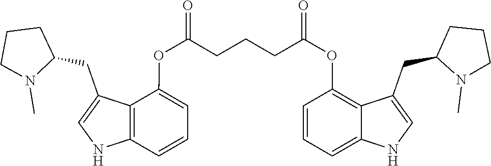 [SMILES: CN1CCC[C@@H]1Cc1c[nH]c2cccc(OC(=O)CCCC(=O)Oc3cccc4[nH]cc(C[C@H]5CCCN5C)c34)c12] |
| 1-14 | 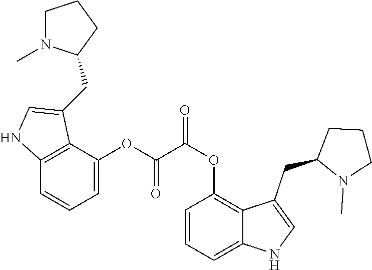 [SMILES: CN1CCC[C@@H]1Cc1c[nH]c2cccc(OC(=O)C(=O)Oc3cccc4[nH]cc(C[C@H]5CCCN5C)c34)c12] |
| 1-15 | 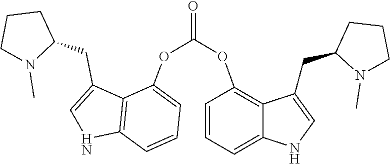 [SMILES: CN1CCC[C@@H]1Cc1c[nH]c2cccc(OC(=O)Oc3cccc4[nH]cc(C[C@H]5CCCN5C)c34)c12] |
| 1-16 | 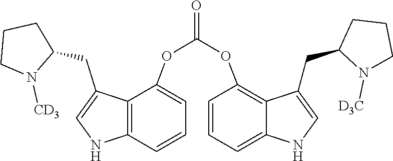 [SMILES: [2H]C([2H])([2H])N1CCC[C@@H]1Cc1c[nH]c2cccc(OC(=O)Oc3cccc4[nH]cc(C[C@H]5CCCN5C([2H])([2H])[2H])c34)c12] |
| 1-17 | 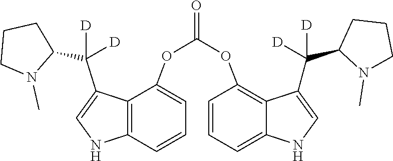 [SMILES: [2H]C([2H])(c1c[nH]c2cccc(OC(=O)Oc3cccc4[nH]cc(C([2H])([2H])[C@H]5CCCN5C)c34)c12)[C@H]1CCCN1C] |
| 1-18 | 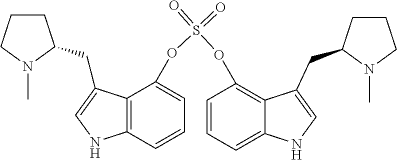 [SMILES: CN1CCC[C@@H]1Cc1c[nH]c2cccc(OS(=O)(=O)Oc3cccc4[nH]cc(C[C@H]5CCCN5C)c34)c12] |
| 1-19 | 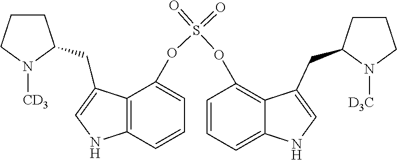 [SMILES: [2H]C([2H])([2H])N1CCC[C@@H]1Cc1c[nH]c2cccc(OS(=O)(=O)Oc3cccc4[nH]cc(C[C@H]5CCCN5C([2H])([2H])[2H])c34)c12] |
| 1-20 | 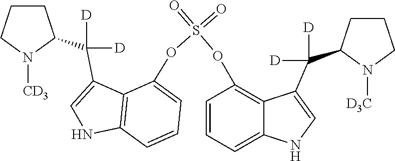 [SMILES: [2H]C([2H])(c1c[nH]c2cccc(OS(=O)(=O)Oc3cccc4[nH]cc(C([2H])([2H])[C@H]5CCCN5C([2H])([2H])[2H])c34)c12)[C@H]1CCCN1C([2H])([2H])[2H]] |
| 1-21 | 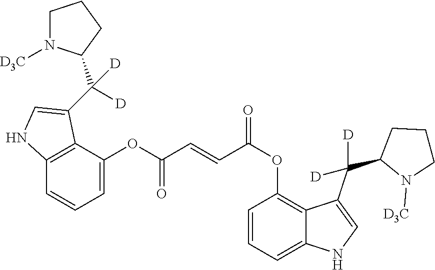 [SMILES: [2H]C([2H])(c1c[nH]c2cccc(OC(=O)/C=C/C(=O)Oc3cccc4[nH]cc(C([2H])([2H])[C@H]5CCCN5C([2H])([2H])[2H])c34)c12)[C@H]1CCCN1C([2H])([2H])[2H]] |
| 1-22 | 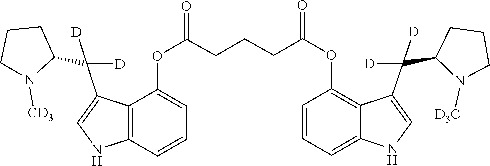 [SMILES: [2H]C([2H])(c1c[nH]c2cccc(OC(=O)CCCC(=O)Oc3cccc4[nH]cc(C([2H])([2H])[C@H]5CCCN5C([2H])([2H])[2H])c34)c12)[C@H]1CCCN1C([2H])([2H])[2H]] |
| 1-23 | 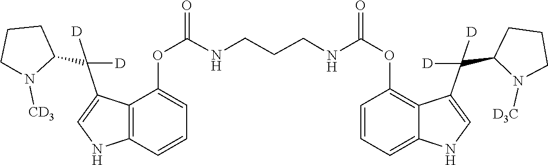 [SMILES: [2H]C([2H])(c1c[nH]c2cccc(OC(=O)NCCCNC(=O)Oc3cccc4[nH]cc(C([2H])([2H])[C@H]5CCCN5C([2H])([2H])[2H])c34)c12)[C@H]1CCCN1C([2H])([2H])[2H]] |
| 1-24 | 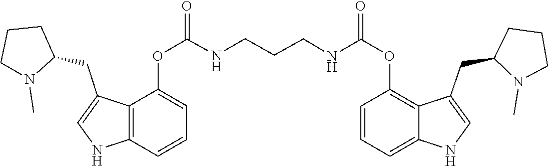 [SMILES: CN1CCC[C@@H]1Cc1c[nH]c2cccc(OC(=O)NCCCNC(=O)Oc3cccc4[nH]cc(C[C@H]5CCCN5C)c34)c12] |
| 1-25 | 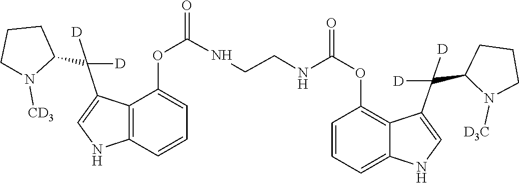 [SMILES: [2H]C([2H])(c1c[nH]c2cccc(OC(=O)NCCNC(=O)Oc3cccc4[nH]cc(C([2H])([2H])[C@H]5CCCN5C([2H])([2H])[2H])c34)c12)[C@H]1CCCN1C([2H])([2H])[2H]] |
| 1-26 | 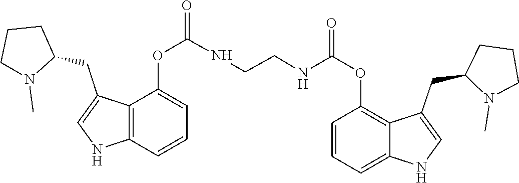 [SMILES: CN1CCC[C@@H]1Cc1c[nH]c2cccc(OC(=O)NCCNC(=O)Oc3cccc4[nH]cc(C[C@H]5CCCN5C)c34)c12] |
| 1-27 | 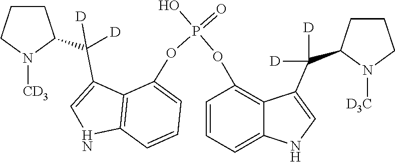 [SMILES: [2H]C([2H])(c1c[nH]c2cccc(OP(=O)(O)Oc3cccc4[nH]cc(C([2H])([2H])[C@H]5CCCN5C([2H])([2H])[2H])c34)c12)[C@H]1CCCN1C([2H])([2H])[2H]] |
| 1-28 | 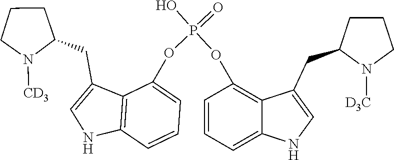 [SMILES: [2H]C([2H])([2H])N1CCC[C@@H]1Cc1c[nH]c2cccc(OP(=O)(O)Oc3cccc4[nH]cc(C[C@H]5CCCN5C([2H])([2H])[2H])c34)c12] |
| 1-29 | 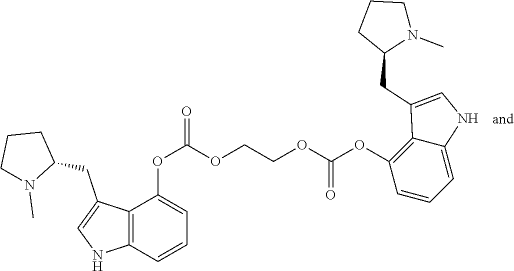 [SMILES: CN1CCC[C@@H]1Cc1c[nH]c2cccc(OC(=O)OCCOC(=O)Oc3cccc4[nH]cc(C[C@H]5CCCN5C)c34)c12 ; "and"] |
| 1-30 | 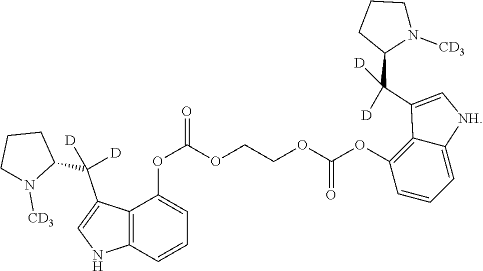 [SMILES: [2H]C([2H])(c1c[nH]c2cccc(OC(=O)OCCOC(=O)Oc3cccc4[nH]cc(C([2H])([2H])[C@H]5CCCN5C([2H])([2H])[2H])c34)c12)[C@H]1CCCN1C([2H])([2H])[2H]] |

or a pharmaceutically acceptable salt, solvate, and/or prodrug thereof.

27. A pharmaceutical composition comprising one or more compounds of claim 1, or a pharmaceutically acceptable salt, solvate, and/or prodrug thereof, and a pharmaceutically acceptable carrier.

28. A method of treating a disease, disorder, or condition comprising administering a therapeutically effective amount of one or more compounds of claim 1, or a pharmaceutically acceptable salt, solvate, and/or prodrug thereof, to a subject having the disease, disorder, or condition.
